(19) United States
(12) Patent Application Publication
(10) Pub. No.: US 2023/0158105 A1
(43) Pub. Date: May 25, 2023

(54) Compositions and uses of Oligo-chromopeptides and methods of making

(71) Applicant: JDS Therapeutics, LLC, Harrison, NY (US)

(72) Inventor: James R. Komorowski, Trumbull, CT (US)

(21) Appl. No.: 17/953,307

(22) Filed: Sep. 26, 2022

Related U.S. Application Data
(60) Provisional application No. 63/296,477, filed on Jan. 4, 2022.
(60) Provisional application No. 63/261,645, filed on Sep. 24, 2021.

(51) Int. Cl. A61K 38/16; C12P 21/06; A61P 39/06; A61P 31/04; A61K 33/04; A61K 33/30; A61P 31/12; A61K 31/706; A23L 33/00; A23L 33/18; A23L 33/16

(52) U.S. Cl. CPC A61K 38/164; C12Y 304/21062; C12P 21/06; A61P 39/06; A61P 31/04; A61K 33/04; A61K 33/30; A61P 31/12; A61K 31/706; A23L 33/40; A23L 33/18; A23L 33/16

(57) ABSTRACT

Processes for producing oligo-chromopeptides from phycocyanin are disclosed. Additionally, compositions comprising oligo-chromopeptides and combination of oligo-chromopeptides with at least one of the compounds namely, nicotinamide riboside, zinc, selenium, or a combination thereof are disclosed. Uses and methods of treating, preventing, or ameliorating age-related somatic disease, bacterial and/or viral infections/diseases, and diseases associated with oxidative stress, or symptoms associated with any of the foregoing are also disclosed.

### CROSS-REFERENCE TO RELATED APPLICATIONS

This application claims the benefit of U.S. Provisional Application No. 63/296,477 filed on Jan. 4, 2022 and U.S. Provisional Application No. 63/261,645 filed on Sep. 24, 2021, the disclosure of each of which is incorporated herein by reference.

### BACKGROUND

Spirulina is a photosynthetic biomass of cyanobacteria (blue-green algae) that contains a number of pigment-proteins (phycobiliproteins), collectively known as phycocyanin (PC). The main phycobiliproteins in PC are C-phycocyanin (C-PC), allophycocyanin (APC), and phycocerythrin (PCE). C-PC and APC bind the chromophore phycocyanobilin (PCB). C-PC comprises a heterodimer with an α- and β-subunit, wherein one PCB molecule covalently binds to the Cys-84 residue of α-subunit, and two PCB molecules covalently bind with Cys-82 and Cys-153 residues of β-subunit. The heterodimer structure and PCB binding of C-PC results in C-PC having an intense blue color in solution and a strong fluorescent profile.

PC, C-PC, and PCB have garnered much attention in the medicine field for potential health benefits. Owing to its highly fluorescent profile C-PC has been utilized as a biomarker, such as those for monitoring aspects of redox and immune status in biological fluids and cells, as well tracing applications. Various studies have also shown that PC, C-PC, and PCB may be useful as a therapeutic for treating or preventing diseases associated with oxidative stress because of its antioxidant, anti-inflammatory, anti-tumor, radical scavenging, and immunomodulatory properties.

Along these lines, oxidative stress has also been recognized as a crucial component in the emergence of mood disorders, such as depression. Depressed individuals are at increased risk of developing age-related somatic diseases, such as coronary heart disease and dementia, and experiencing accelerated aging, which can occur at the cellular level, specifically at the telomere level. Telomeres are nucleoprotein complexes that protect the chromosome ends from fusion and DNA damage by capping the ends of linear DNA. Stress can increase oxidative damage and decrease antioxidant defenses, and oxidative stress has been shown to increase with aging and in various disease states, thus increasing the likelihood of cellular damage. Stress hormones, such as corticosterone (CORT), play an essential role in the development of depression. Various studies have shown that chronic CORT administration induces dysfunction in the hypothalamic-pituitary-adrenal axis leading to depression, which was in turn associated with accelerated aging.

Other research has suggested beneficial outcomes using PC and C-PC to treat or prevent infectious diseases, such as viral and bacterial infections/diseases. Viral diseases are the most common human respiratory diseases, and are known for their high morbidity and mortality, especially in the elderly, and chronically ill people. Viruses cause seasonal epidemics, as well as pandemics that can spread rapidly and infect up to 50% of affected populations. Aside from mortality, viral diseases also inflicts a heavy economic and medical burden on society, and represents a serious public health problem that needs to be effectively addressed. Virus virulence is likely driven by both high viral loads and an overly aggressive host immune response, which combine to induce extensive inflammation and apoptosis. Immunity plays a very important role in preventing these diseases. It is well-known that macrophages play an important role in an immune response by serving as the first-line immunological defense against infections in mammals through the secretion of various cytokines. Cytokines are secreted from various kinds of cells and play a critical role in protection against bacterial and viral infections. Research has shown that PC and C-PC can enhance activity of lymphocytes and the immunity of an organism to prevent and resist diseases, without causing toxicity.

The inventors have discovered, the aforementioned properties of PC, C-PC, and PCB can be further improved by creating and utilizing oligo-chromopeptide fragments (O-CPs) from the digestion of PC in place of PC, C-PC, or PCB. Komorowski et al. “Enhanced Antioxidant Activity of Phycocyanin Oligopeptides,” which is hereby incorporated by reference in its entirety, found that using O-CPs conferred significant antioxidant potency compared to PC, as evaluated in the Folin-Ciocalteu reaction assay and the Oxygen Radical Absorbance Capacity (ORAC) 6.0 assay.

Additionally, the inventors have contemplated the inclusion of additional compounds thought to improve the aforementioned effects of O-CPs.

Such compounds contemplated are minerals such zinc (Zn) and selenium (Se). Dietary Zn deficiency is common globally and appears to contribute to the pathogenesis of many diseases. Besides dietary deficiency, low Zn levels are present in individuals with other abnormalities that either limit Zn absorption, such as in alcoholics, or that cause hepatic Zn sequestration, such as viral or bacterial infections. Zn deficiency produces numerous abnormalities, but is particularly detrimental to epithelial cells and the immune system. For example, higher incidence of bacterial infections was reported in HIV-1-infected persons with low Zn levels, and lower levels of Zn correlated with more advanced stages of the disease. Se is a nutritionally essential trace mineral that plays a vital role in various inflammatory and immune responses. Se compounds have been reported to regulate the function of neutrophils, natural killer (NK) cells, B lymphocytes, and T cells and affect the incorporation of Se into important immune organs. Supplementation with Se suppresses the acute inflammatory response in mice and promotes immune function by alleviating the expression of Lipopolysaccharide (LPS) induced pro-inflammatory cytokines, such as interleukin-6 (IL-6) and prostaglandin-endoperoxide synthase 2 (COX-2). LPS is a structural and functional component of the cell wall of gram negative bacteria made up of an outer part (O) polysaccharide, the core and a lipid component, lipid A. LPS activates cells of the innate immune system by binding to toll-like receptor 4 (TLR4) and commences a hyper-inflammatory state similar to sepsis.

Another compound contemplated is nicotinamide riboside (NR); NR is a natural precursor for nicotinamide adenine dinucleotide (NAD+) biosynthesis. NAD+ levels play a crucial role in energy homeostasis and genome stability by acting as a substrate for different families of enzymes, such as sirtuins (SIRTs) and poly(ADP-ribose) polymerases (PARPs). NAD+ bioavailability declines in situations of metabolic disease, including diet-induced obesity and non-alcoholic fatty liver disease (NAFLD), as well as during aging, fostering age-related physiological decline. NAD+'s ability to sensitize insulin and induce mitochondrial biogenesis suggests that it could find meaningful applications in the treatment of metabolic disorders and neurodegenerative disease.

While O-CPs have demonstrated some therapeutic promise, there are significant challenges associated with making and isolating O-CPs from PC, C-PC, or PCB.

The processes for making and isolating O-CPs from PC, C-PC, or PCB include high pressure homogenization, acid treatment, enzymatic treatment (by lysozyme), organic solvent extraction, and sonication, to name a few. In some instances, these processes require the use of hazardous chemicals and have a low extraction efficiency. In other instances, the processes have low filtration rates owing to the presence of suspended particles, which renders the processes lengthy and cumbersome. The challenges in filtration, centrifugation, and purification of the final product make the existing processes unfeasible for commercial scale production.

The processes for making O-CPs from PCB include those disclosed by McCarty et al. (U.S. Pat. No. 8,709,434) and for making O-CPs from C-PC by Minic et al. “Digestion by pepsin releases biologically active chromopeptides from C-phycocyanin, a blue-colored biliprotein of microalga Spirulina,” both of which are hereby incorporated by reference in their entirety. McCarty discloses a method of making O-CPs, wherein the method comprises extraction of PCB from Spirulina using hot methanol, filtration, and reacted with methanol containing ascorbic acid at 60° C. for 8 hr. These conditions result in the thioether bonds linking PCB to PC apoprotein being cleaved via methanolysis. The reaction product was then subjected to a workup in accordance with standard organic chemistry steps known in the prior art. Minic discloses a method of making O-CPs, wherein the method comprises extraction of C-PC in phosphate buffer, followed by digestion of C-PC with pepsin in simulated gastric fluid ((SGF) comprising ˜0.1M HCl, pH 1.2). These methods of the prior art, however, fail to provide a reliable method for making O-CPs that would be compatible with large-scale manufacturing, thus limiting these to the academic setting. The use of methanol, a hazardous and flammable solvent, in McCarty, and the reliance of strong acid conditions (SGF), in Minic, are incompatible with conditions associated with large scale manufacturing; removal of methanol is a costly endeavor to ensure safety of the final product and HCl is incompatible with steel and would require specialized equipment to operate on a large scale. Furthermore, in the context of nutritional supplements, drugs, vitamins, or other items to be ingested by humans, the prior art processes result in the likelihood of residual solvents or harsh chemicals that are potentially damaging to the environment or that are harmful or unsafe for human consumption.

The inventors have identified a need for an improved, economically feasible, and large-scale manufacturing-compatible process for the extraction and production of O-CPs from Spirulina or PC with high yield, which can further be used for medicinal purposes. The inventors have also identified that O-CPs can provide better therapeutic benefits than their PC counterparts. Both the method of making O-CPs, avoiding the use of methanol and HCl, and their therapeutic use provide superior, and unexpected benefits, compared to known methods and compounds of the prior art. This finding is novel. As such, by making O-CPs according to the methods disclosed herein and providing O-CPs as pharmaceutical agents and/or dietary supplements, therapeutic and nutraceutical benefits can be realized, either individually, collectively, or in conjunction with other pharmaceutical agents and/or dietary supplements.

### SUMMARY

Embodiments of the present disclosure provide methods for producing oligo-chromopeptides (O-CPs) derived from phycocyanin (PC). Methods as described herein, provide increased production efficiency of O-CPs from PC, higher ultrafiltration rates and shortened processes of production of O-CPs from PC, overcome other challenges associated with productions of O-CPs from PC, C-PC, or PCB by improving digestion, filtration, centrifugation, purification, and avoiding using solvents incompatible with large scale manufacturing, thereby providing feasible methods for commercial scale production. Embodiments of the present disclosure meet the needs for treating, preventing, or ameliorating age-related somatic disease, bacterial and/or viral infections/diseases, and diseases associated with oxidative stress in a subject in need thereof, wherein compositions and methods are provided.

Embodiments of the present disclosure also provide compositions for treating, preventing, or ameliorating age-related somatic disease, bacterial and/or viral infections/diseases, and diseases associated with oxidative stress, or symptoms associated with any of the foregoing in a subject in need thereof.

These and other features, aspects, and advantages of the present embodiments will become understood with reference to the following description, appended claims, and accompanying figures.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 exemplifies a flow chart illustrating a process for producing O-CPs from PC;

FIG. 2 is a graph illustrating the absorption spectrum of a complete digest of PC in 1 M HCl, showing absorption between 500 and 700 nm;

FIG. 3A is a graph illustrating the calibration curve for photometric determination of O-CPs expressed in terms of PC content;

FIG. 3B is a graph illustrating the calibration curve for photometric determination of O-CPs expressed in terms of O-CP content;

FIG. 4A is a graph illustrating complete chromatograms detected at 280 nm and 615 nm;

FIG. 4B is a graph illustrating chromatograms detected at 280 nm and 615 nm in a range for 10-16 minutes that contains O-CP peaks;

FIG. 5 is a graph illustrating HPLC chromatographs for complete digests of PC based on absorption at 615 nm;

FIG. 6A is a graph illustrating the calibration curves for the quantification of O-CPs by HPLC expressed in terms of PC content;

FIG. 6B is a graph illustrating the calibration curves for the quantification of O-CPs by HPLC expressed in terms of O-CPs content;

FIG. 7A is a graph illustrating temporal degradation of PC at 52 g/L in various temperature, pH, and enzymatic conditions, in cases of enzyme present the enzyme is contained at 2 g enzyme/kg of PC;

FIG. 7B is a graph illustrating temporal degradation of PC at 105 g/L in various temperature, pH, and enzymatic conditions, in cases of enzyme present the enzyme is contained at 2 g enzyme/kg of PC;

FIG. 7C is a graph illustrating temporal degradation of PC at 210 g/L in various temperature, pH, and enzymatic conditions, in cases of enzyme present the enzyme is contained at 2 g enzyme/kg of PC;

FIG. 7D is a graph illustrating temporal degradation of PC at 50 g/L in various temperature, pH, and enzymatic conditions, in cases of enzyme present the enzyme is contained at 2 g enzyme/kg of PC;

FIG. 7E is a graph illustrating temporal degradation of PC at 100 g/L in various temperature, pH, and enzymatic conditions, in cases of enzyme present the enzyme is contained at 4 g enzyme/kg of PC;

FIG. 7F is a graph illustrating temporal degradation of PC at 200 g/L in various temperature, pH, and enzymatic conditions, in cases of enzyme present the enzyme is contained at 8 g enzyme/kg of PC;

FIG. 8A is a graph illustrating temporal degradation of PC at 50 g/L at 65° C. and pH 8 for various quantities of alcalase enzyme;

FIG. 8B is a graph illustrating temporal degradation of PC at 50 g/L at 50° C. and pH 8 for various quantities of alcalase enzyme;

FIG. 8C is a graph illustrating temporal degradation of PC at 100 g/L at 65° C. and pH 8 for various quantities of alcalase enzyme;

FIG. 8D is a graph illustrating temporal degradation of PC at 100 g/L at 50° C. and pH 8 for various quantities of alcalase enzyme;

FIG. 8E is a graph illustrating temporal degradation of PC at 200 g/L at 65° C. and pH 8 for various quantities of alcalase enzyme;

FIG. 8F is a graph illustrating temporal degradation of PC at 200 g/L at 50° C. and pH 8 for various quantities of alcalase enzyme;

FIG. 8G is a graph illustrating temporal degradation of PC at 300 g/L at 65° C. and pH 8 for various quantities of alcalase enzyme;

FIG. 8H is a graph illustrating temporal degradation of PC at 300 g/L at 50° C. and pH 8 for various quantities of alcalase enzyme;

FIG. 9 is a graph illustrating ORAC of three different PC sources (1-PC, 2-PC, and 3-PC), O-CPs made by subjecting PC to alcalase enzyme (0.2 g/g) (1-O-CPs (A), 2-O-CPs (A), and 3-O-CPs (A)), and O-CPs made by subjecting PC to alcalase enzyme (0.2 g/g) and viscozyme (0.2 g/g) (1-O-CPs (A+V), 2-O-CPs (A+V), and 3-O-CPs (A+V));

FIG. 10 is a graph illustrating ORAC of PC (PC), O-CPs made by subjecting PC to no enzyme at 65° C. and pH 8 for 5 hr (O-CPs-C1), O-CPs made by subjecting PC to alcalase enzyme (0.1 g/g) at 65° C. and pH 8 for 5 hr (O-CPs-E1), and O-CPs made by subjecting PC to alcalase enzyme (0.2 g/g) at 65° C. and pH 8 for 5 hr (O-CPs-E2);

FIG. 11 is a graph illustrating the total antioxidant capacity of (A) PC and O-CPs, (B) EpiCor, and (C) Wellmune;

FIG. 12 is a graph illustrating the dose response of PC, O-CPs, EpiCor, and Wellmune in peripheral blood mononuclear cells (PBMC);

FIG. 13 shows flow cytometry data showing gates for lymphocytes, monocytes, and the four subsets of lymphocytes, allowing analysis of CD69 expression on all five cell types;

FIG. 14 are graphs illustrating the dose response effects of PC, O-CPs, EpiCor, and Wellmune on expression of CD69 on Natural Killer cells (NK cells) following—direct effects wherein raw data are shown as the average±standard deviation of triplicate cultures;

FIG. 15 are graphs illustrating the dose response effects of PC, O-CPs, EpiCor, and Wellmune on the expression of CD25 on NK cells—direct effects wherein raw data are shown as the average±standard deviation of triplicate cultures;

FIG. 16 are graphs illustrating the dose response effects of PC, O-CPs, EpiCor, and Wellmune on expression of CD69 on Natural killer T (NKT) cells—direct effects wherein raw data are shown as the average±standard deviation of triplicate cultures;

FIG. 17 are graphs illustrating the dose response effects of PC, O-CPs, EpiCor, and Wellmune on expression of CD25 on NKT cells—direct effects wherein raw data are shown as the average±standard deviation of triplicate cultures;

FIG. 18 are graphs illustrating the dose response effects of PC, O-CPs, EpiCor, and Wellmune on expression of CD69 on T cells—direct effects wherein raw data are shown as the average±standard deviation of triplicate cultures;

FIG. 19 are graphs illustrating the dose response effects of PC, O-CPs, EpiCor, and Wellmune on expression of CD25 on T cells—direct effects wherein raw data are shown as the average±standard deviation of triplicate cultures;

FIG. 20 are graphs illustrating the dose response effects of PC, O-CPs, EpiCor, and Wellmune on expression of CD69 on cells that are not NK or T cells—direct effects wherein raw data are shown as the average±standard deviation of triplicate cultures;

FIG. 21 are graphs illustrating the dose response effects of PC, O-CPs, EpiCor, and Wellmune on expression of CD25 on cells that are not NK or T cells—direct effects wherein raw data are shown as the average±standard deviation of triplicate cultures;

FIG. 22 are graphs illustrating the dose response effects of PC, O-CPs, EpiCor, and Wellmune on expression of CD69 on monocytes—direct effects wherein raw data are shown as the average±standard deviation of triplicate cultures;

FIG. 23 are graphs illustrating the dose response effects of PC, O-CPs, EpiCor, and Wellmune on the number of CD69+ and CD25+ lymphocytes—direct effects wherein raw data are shown as the average±standard deviation of triplicate cultures;

FIG. 24 are graphs illustrating the dose response effects of PC, O-CPs, EpiCor, and Wellmune on expression of CD69 on NK cells—LPS induced inflammation wherein the percent change compared to LPS treated control cultures, data are shown as the average±standard deviation of triplicate cultures;

FIG. 25 are graphs illustrating the dose response effects of PC, O-CPs, EpiCor, and Wellmune on expression of CD25 on NK cells—LPS induced inflammation wherein the percent change compared to LPS treated control cultures, data are shown as the average±standard deviation of triplicate cultures;

FIG. 26 are graphs illustrating the dose response effects of PC, O-CPs, EpiCor, and Wellmune on expression of CD69 on NKT cells—LPS induced inflammation wherein the percent change compared to LPS treated control cultures, data are shown as the average±standard deviation of triplicate cultures;

FIG. 27 are graphs illustrating the dose response effects of PC, O-CPs, EpiCor, and Wellmune on expression of CD25 on NKT cells—LPS induced inflammation wherein the percent change compared to LPS treated control cultures, data are shown as the average±standard deviation of triplicate cultures;

FIG. 28 are graphs illustrating the dose response effects of PC, O-CPs, EpiCor, and Wellmune on expression of CD69 on T cells—LPS induced inflammation wherein the percent change compared to LPS treated control cultures, data are shown as the average±standard deviation of triplicate cultures;

FIG. 29 are graphs illustrating the dose response effects of PC, O-CPs, EpiCor, and Wellmune on expression of CD25 on T cells—LPS induced inflammation wherein the percent change compared to LPS treated control cultures, data are shown as the average±standard deviation of triplicate cultures;

FIG. 30 are graphs illustrating the dose response effects of PC, O-CPs, EpiCor, and Wellmune on expression of CD69 on cells that are not NK or T cells—LPS induced inflammation wherein the percent change compared to LPS treated control cultures, data are shown as the average±standard deviation of triplicate cultures;

FIG. 31 are graphs illustrating the dose response effects of PC, O-CPs, EpiCor, and Wellmune on expression of CD25 on cells that are not NK or T cells—LPS induced inflammation wherein the percent change compared to LPS treated control cultures, data are shown as the average±standard deviation of triplicate cultures;

FIG. 32 are graphs illustrating the dose response effects of PC, O-CPs, EpiCor, and Wellmune on expression of CD69 on monocytes—LPS induced inflammation wherein the percent change compared to LPS treated control cultures, data are shown as the average±standard deviation of triplicate cultures;

FIG. 33 are graphs illustrating the dose response effects of PC, O-CPs, EpiCor, and Wellmune on the number of CD69+ and CD25+ lymphocytes—LPS induced inflammation wherein the percent change compared to LPS treated control cultures, data are shown as the average±standard deviation of triplicate cultures;

FIG. 34 are graphs illustrating the dose response effects of PC, O-CPs, EpiCor, and Wellmune on expression of CD69 on NK cells—Polyinosinic:polycytidylic acid (Poly I:C) immune activation, data are shown as the average±standard deviation of triplicate cultures;

FIG. 35 are graphs illustrating the dose response effects of PC, O-CPs, EpiCor, and Wellmune on expression of CD25 on NK cells—Poly I:C immune activation, data are shown as the average±standard deviation of triplicate cultures;

FIG. 36 are graphs illustrating the dose response effects of PC, O-CPs, EpiCor, and Wellmune on expression of CD69 on NKT cells—Poly I:C immune activation, data are shown as the average±standard deviation of triplicate cultures;

FIG. 37 are graphs illustrating the dose response effects of PC, O-CPs, EpiCor, and Wellmune on expression of CD25 on NKT cells—Poly I:C immune activation, data are shown as the average±standard deviation of triplicate cultures;

FIG. 38 are graphs illustrating the dose response effects of PC, O-CPs, EpiCor, and Wellmune on expression of CD69 on T cells—Poly I:C immune activation, data are shown as the average±standard deviation of triplicate cultures;

FIG. 39 are graphs illustrating the dose response effects of PC, O-CPs, EpiCor, and Wellmune on expression of CD25 on T cells—Poly I:C immune activation, data are shown as the average±standard deviation of triplicate cultures;

FIG. 40 are graphs illustrating the dose response effects of PC, O-CPs, EpiCor, and Wellmune on expression of CD69 on cells that are not NK or T cells—Poly I:C immune activation, data are shown as the average±standard deviation of triplicate cultures;

FIG. 41 are graphs illustrating the dose response effects of PC, O-CPs, EpiCor, and Wellmune on expression of CD25 on cells that are not NK or T cells—Poly I:C immune activation, data are shown as the average±standard deviation of triplicate cultures;

FIG. 42 are graphs illustrating the dose response effects of PC, O-CPs, EpiCor, and Wellmune on expression of CD69 on monocytes—Poly I:C immune activation, data are shown as the average±standard deviation of triplicate cultures;

FIG. 43 are graphs illustrating the dose response effects of PC, O-CPs, EpiCor, and Wellmune on the number of CD69+ and CD25+ lymphocytes—Poly I:C immune activation, data are shown as the average±standard deviation of triplicate cultures;

FIG. 44 are graphs illustrating the dose response effects of PC, O-CPs, EpiCor, and Wellmune on expression of interleukin-1-beta (IL-1β) levels in PBMC cultures—direct effects, data are shown as the average±standard deviation of triplicate cultures;

FIG. 45 are graphs illustrating the dose response effects of PC, O-CPs, EpiCor, and Wellmune on expression of interleukin-6 (IL-6) levels in PBMC cultures—direct effects, data are shown as the average±standard deviation of triplicate cultures;

FIG. 46 are graphs illustrating the dose response effects of PC, O-CPs, EpiCor, and Wellmune on expression of interleukin-8 (IL-8) levels in PBMC cultures—direct effects, data are shown as the average±standard deviation of triplicate cultures;

FIG. 47 are graphs illustrating the dose response effects of PC, O-CPs, EpiCor, and Wellmune on expression of interleukin-10 (IL-10) levels in PBMC cultures—direct effects, data are shown as the average±standard deviation of triplicate cultures;

FIG. 48 are graphs illustrating the dose response effects of PC, O-CPs, EpiCor, and Wellmune on expression of interleukin-1 receptor antagonist (IL-1ra) levels in PBMC cultures—direct effects, data are shown as the average±standard deviation of triplicate cultures;

FIG. 49 are graphs illustrating the dose response effects of PC, O-CPs, EpiCor, and Wellmune on expression of macrophage inflammatory protein 1 alpha (CCL3) (MIP-1α) levels in PBMC cultures—direct effects, data are shown as the average±standard deviation of triplicate cultures;

FIG. 50 are graphs illustrating the dose response effects of PC, O-CPs, EpiCor, and Wellmune on expression of macrophage inflammatory protein 1 beta (CCL4) (MIP-1β) levels in PBMC cultures—direct effects, data are shown as the average±standard deviation of triplicate cultures;

FIG. 51 are graphs illustrating the dose response effects of PC, O-CPs, EpiCor, and Wellmune on expression of interferon-gamma (IFN-γ) levels in PBMC cultures—direct effects, data are shown as the average±standard deviation of triplicate cultures;

FIG. 52 are graphs illustrating the dose response effects of PC, O-CPs, EpiCor, and Wellmune on expression of tumor necrosis factor alpha (TNF-α) levels in PBMC cultures—direct effects, data are shown as the average±standard deviation of triplicate cultures;

FIG. 53 are graphs illustrating the dose response effects of PC, O-CPs, EpiCor, and Wellmune on expression of granulocyte colony-stimulating factor (G-CSF) levels in PBMC cultures—direct effects, data are shown as the average±standard deviation of triplicate cultures;

FIG. 54 are graphs illustrating the dose response effects of PC, O-CPs, EpiCor, and Wellmune on expression of IL-1β levels in PBMC cultures—LPS induced inflammation wherein the percent change compared to LPS treated control cultures, data are shown as the average±standard deviation of triplicate cultures;

FIG. 55 are graphs illustrating the dose response effects of PC, O-CPs, EpiCor, and Wellmune on expression of IL-6 levels in PBMC cultures—LPS induced inflammation wherein the percent change compared to LPS treated control cultures, data are shown as the average±standard deviation of triplicate cultures;

FIG. 56 are graphs illustrating the dose response effects of PC, O-CPs, EpiCor, and Wellmune on expression of IL-8 levels in PBMC cultures—LPS induced inflammation wherein the percent change compared to LPS treated control cultures, data are shown as the average±standard deviation of triplicate cultures;

FIG. 57 are graphs illustrating the dose response effects of PC, O-CPs, EpiCor, and Wellmune on expression of IL-10 levels in PBMC cultures—LPS induced inflammation wherein the percent change compared to LPS treated control cultures, data are shown as the average±standard deviation of triplicate cultures;

FIG. 58 are graphs illustrating the dose response effects of PC, O-CPs, EpiCor, and Wellmune on expression of IL-1ra levels in PBMC cultures—LPS induced inflammation wherein the percent change compared to LPS treated control cultures, data are shown as the average±standard deviation of triplicate cultures;

FIG. 59 are graphs illustrating the dose response effects of PC, O-CPs, EpiCor, and Wellmune on expression of MIP-1α levels in PBMC cultures—LPS induced inflammation wherein the percent change compared to LPS treated control cultures, data are shown as the average±standard deviation of triplicate cultures;

FIG. 60 are graphs illustrating the dose response effects of PC, O-CPs, EpiCor, and Wellmune on expression of MIP-1β levels in PBMC cultures—LPS induced inflammation wherein the percent change compared to LPS treated control cultures, data are shown as the average±standard deviation of triplicate cultures;

FIG. 61 are graphs illustrating the dose response effects of PC, O-CPs, EpiCor, and Wellmune on expression of IFN-γ levels in PBMC cultures—LPS induced inflammation wherein the percent change compared to LPS treated control cultures, data are shown as the average±standard deviation of triplicate cultures;

FIG. 62 are graphs illustrating the dose response effects of PC, O-CPs, EpiCor, and Wellmune on expression of TNF-α levels in PBMC cultures—LPS induced inflammation wherein the percent change compared to LPS treated control cultures, data are shown as the average±standard deviation of triplicate cultures;

FIG. 63 are graphs illustrating the dose response effects of PC, O-CPs, EpiCor, and Wellmune on expression of G-CSF levels in PBMC cultures—LPS induced inflammation wherein the percent change compared to LPS treated control cultures, data are shown as the average±standard deviation of triplicate cultures;

FIG. 64 are graphs illustrating the dose response effects of PC, O-CPs, EpiCor, and Wellmune on expression of IL-1β levels in PBMC cultures—Poly I:C immune activation, data are shown as the average±standard deviation of triplicate cultures;

FIG. 65 are graphs illustrating the dose response effects of PC, O-CPs, EpiCor, and Wellmune on expression of IL-6 levels in PBMC cultures—Poly I:C immune activation, data are shown as the average±standard deviation of triplicate cultures;

FIG. 66 are graphs illustrating the dose response effects of PC, O-CPs, EpiCor, and Wellmune on expression of IL-8 levels in PBMC cultures—Poly I:C immune activation, data are shown as the average±standard deviation of triplicate cultures;

FIG. 67 are graphs illustrating the dose response effects of PC, O-CPs, EpiCor, and Wellmune on expression of IL-10 levels in PBMC cultures—Poly I:C immune activation, data are shown as the average±standard deviation of triplicate cultures;

FIG. 68 are graphs illustrating the dose response effects of PC, O-CPs, EpiCor, and Wellmune on expression of IL-1ra levels in PBMC cultures—Poly I:C immune activation, data are shown as the average±standard deviation of triplicate cultures;

FIG. 69 are graphs illustrating the dose response effects of PC, O-CPs, EpiCor, and Wellmune on expression of MIP-1α levels in PBMC cultures—Poly I:C immune activation, data are shown as the average±standard deviation of triplicate cultures;

FIG. 70 are graphs illustrating the dose response effects of PC, O-CPs, EpiCor, and Wellmune on expression of MIP-1β levels in PBMC cultures—Poly I:C immune activation, data are shown as the average±standard deviation of triplicate cultures;

FIG. 71 are graphs illustrating the dose response effects of PC, O-CPs, EpiCor, and Wellmune on expression of IFN-γ levels in PBMC cultures—Poly I:C immune activation, data are shown as the average±standard deviation of triplicate cultures;

FIG. 72 are graphs illustrating the dose response effects of PC, O-CPs, EpiCor, and Wellmune on expression of TNF-α levels in PBMC cultures—Poly I:C immune activation, data are shown as the average±standard deviation of triplicate cultures;

FIG. 73 are graphs illustrating the dose response effects of PC, O-CPs, EpiCor, and Wellmune on expression of G-CSF levels in PBMC cultures—Poly I:C immune activation, data are shown as the average±standard deviation of triplicate cultures;

FIG. 74 is a graph illustrating the effects of 30 mg or 300 mg human equivalent dose (HED) of O-CPs, O-CP30 or O-CP300, respectively, 300 mg HED of nicotinamide riboside (NR), and their combination on telomere length in corticosterone-(CORT) induced aging rats Different symbols (a-f) indicate statistical differences among the groups (ANOVA and Tukey's post-hoc test; P<0.05);

FIG. 75 are graphs illustrating the effects of O-CPs, NR, and their combination on (A) liver IL-6 levels, (B) liver TNF-α levels, (C) liver IL-1β levels, and (D) liver IL-8 levels in CORT-induced aging rats. Different symbols (a-f) indicate statistical differences among the groups (ANOVA and Tukey's post-hoc test; P<0.05);

FIG. 76 are graphs illustrating the effects of O-CPs, NR, and their combination on (A) liver protection of telomeres protein 1-a (POT1a) levels, (B) liver protection of telomeres protein 1-b (POT1b) levels, (C) liver telomeric repeat-binding factor 1 (TRF1) levels, and (D) liver telomeric repeat-binding factor 2 (TRF2) levels in CORT-induced aging rats. Different symbols (a-e) indicate statistical differences among the groups (ANOVA and Tukey's post-hoc test; P<0.05);

FIG. 77 is a graph illustrating the effects of O-CPs, NR, and their combination on liver TRF1-interacting nuclear factor 2 (Tin2) levels in CORT-induced aging rats. Different symbols (a-f) indicate statistical differences among the groups (ANOVA and Tukey's post-hoc test; P<0.05);

FIG. 78 are graphs illustrating the effects of O-CPs, NR, and their combination on (A) liver sirtuin 1 (SIRT1) levels, (B) liver sirtuin 3 (SIRT3) levels, and (C) liver nicotinamide phosphoribosyltransferase (NAMPT) levels in CORT-induced aging rats. Different symbols (a-e) indicate statistical differences among the groups (ANOVA and Tukey's post-hoc test; P<0.05);

FIG. 79 are graphs illustrating the effects of O-CPs, NR, and their combination on (A) brain nuclear factor kappa B (NF-κB) levels and (B) brain nuclear factor erythroid 2-related factor 2 (Nrf2) levels in CORT-induced aging rats. Different symbols (a-f) indicate statistical differences among the groups (ANOVA and Tukey's post-hoc test; P<0.05);

FIG. 80 are graphs illustrating the effects of O-CPs, NR, and their combination on (A) brain brain-derived neurotrophic factor (BDNF) levels, (B) brain nerve growth factor (NGF) levels, (C) brain postsynaptic density-93 (PSD93) levels, and (D) brain postsynaptic density-95 (PSD95) levels in CORT-induced aging rats. Different symbols (a-e) indicate statistical differences among the groups (ANOVA and Tukey's post-hoc test; P<0.05);

FIG. 81 is a graph illustrating the effects of O-CPs, NR, and their combination on brain synapsin I levels in CORT-induced aging rats. Different symbols (a-e) indicate statistical differences among the groups (ANOVA and Tukey's post-hoc test; P<0.05);

FIG. 82 are graphs illustrating the effects of 200 μg HED of Se, 30 mg HED of Zn, 300 mg HED of O-CPs (PC-0), and their combination on (A) temporal febrile response, and (B) total pyrexia in LPS-induced mice;

FIG. 83 are graphs illustrating the effects of Se, Zn, O-CPs, and their combination on (A) serum alanine transaminase (ALT) levels, (B) serum aspartate transaminase (AST) levels, (C) serum alkaline phosphatase (ALP) levels, and (D) serum lactate dehydrogenase (LDH) levels in LPS-induced mice. Different symbols (a-e) indicate statistical differences among the groups (ANOVA and Tukey's post-hoc test; P<0.05);

FIG. 84 are graphs illustrating the effects of Se, Zn, O-CPs, and their combination on (A) serum IL-1β levels, (B) serum IL-6 levels, (C) serum TNF-α levels, and (D) serum receptor tyrosine-protein kinase erbB-2 (Neu) levels in LPS-induced mice. Different symbols (a-e) indicate statistical differences among the groups (ANOVA and Tukey's post-hoc test; P<0.05);

FIG. 85 are graphs illustrating the effects of Se, Zn, O-CPs, and their combination on (A) liver Zn levels, (B) liver Se levels, (C) lung Zn levels, and (D) lung Se levels in LPS-induced mice. Different symbols (a-c) indicate statistical differences among the groups (ANOVA and Tukey's post-hoc test; P<0.05);

FIG. 86 are histological images showing the effects of Se, Zn, O-CPs, and their combination on lung damage in LPS-induced mice;

FIG. 87 are histological images showing the effects of Se, Zn, O-CPs, and their combination on liver damage in LPS-induced mice;

FIG. 88 are graphs illustrating the effects of Se, Zn, O-CPs, and their combination on (A) liver IL-1β levels, (B) liver IL-6 levels, (C) liver interleukin-17 (IL-17) levels, (D) liver TNF-α levels, (E) liver NF-κB levels, (F) liver COX-2 levels, and (G) inducible nitric oxide synthase (iNOS) levels in LPS-induced mice. Different symbols (a-e) indicate statistical differences among the groups (ANOVA and Tukey's post-hoc test; P<0.05);

FIG. 89 are graphs illustrating the effects of Se, Zn, O-CPs, and their combination on (A) liver metallothionein (MT) levels, (B) liver zinc transporter ZIP1 (ZIP1) levels, (C) liver zinc transporter ZIP4 (ZIP4) levels, (D) liver zinc transporter ZIP14 (ZIP14) levels, and (E) liver zinc transporter 1 (ZNT1) levels in LPS-induced mice. Different symbols (a-d) indicate statistical differences among the groups (ANOVA and Tukey's post-hoc test; P<0.05);

FIG. 90 are graphs illustrating the effects of Se, Zn, O-CPs, and their combination on (A) lung IL-1β levels, (B) lung IL-6 levels, (C) lung IL-17 levels, (D) lung TNF-α levels, (E) lung NF-κB levels, (F) lung COX-2 levels, and (G) lung iNOS levels in LPS-induced mice. Different symbols (a-e) indicate statistical differences among the groups (ANOVA and Tukey's post-hoc test; P<0.05); and

FIG. 91 are graphs illustrating the effects of Se, Zn, O-CPs, and their combination on (A) lung MT levels, (B) lung ZIP1 levels, (C) lung ZIP4 levels, (D) lung ZIP14 levels, (E) lung ZNT1 levels, and (F) lung zinc transporter 4 (ZNT4) levels in LPS-induced mice. Different symbols (a-d) indicate statistical differences among the groups (ANOVA and Tukey's post-hoc test; P<0.05).

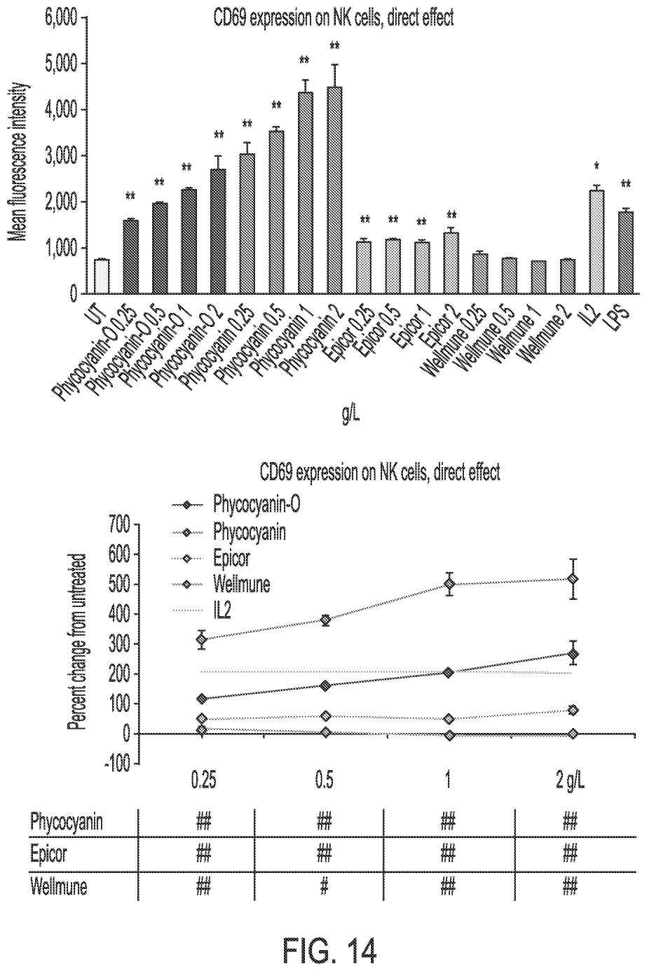

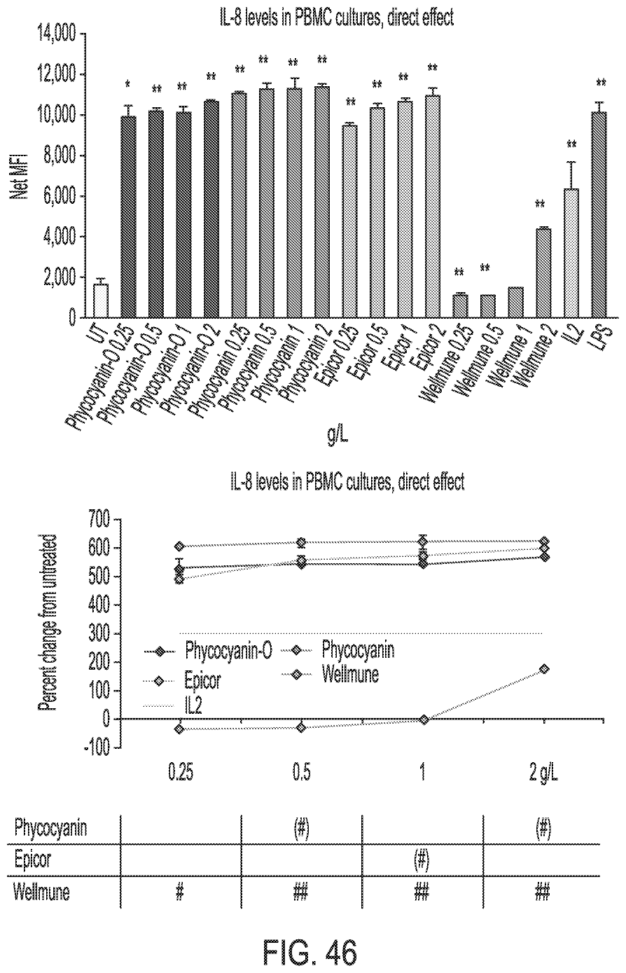

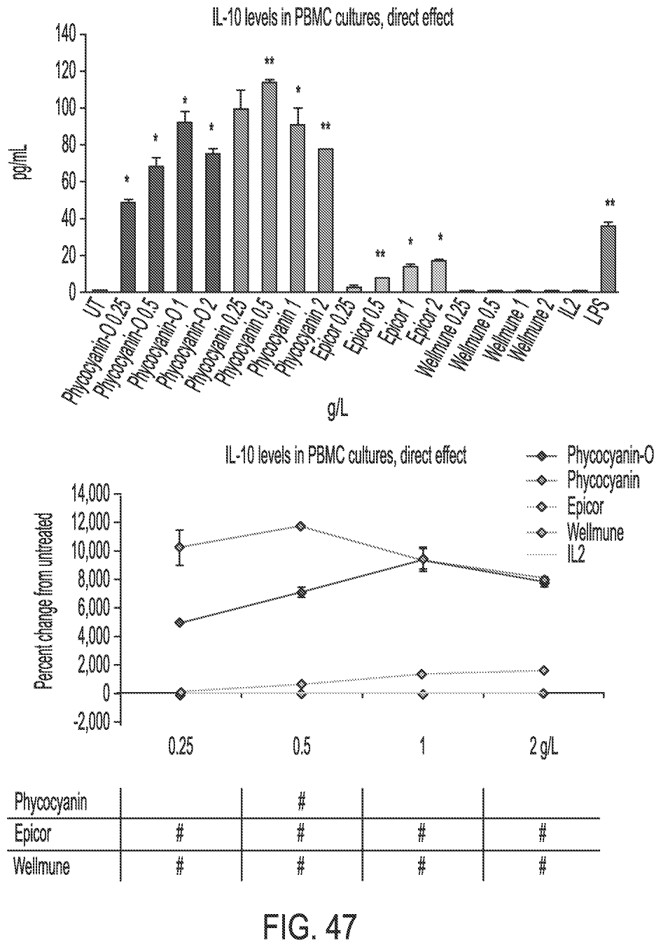

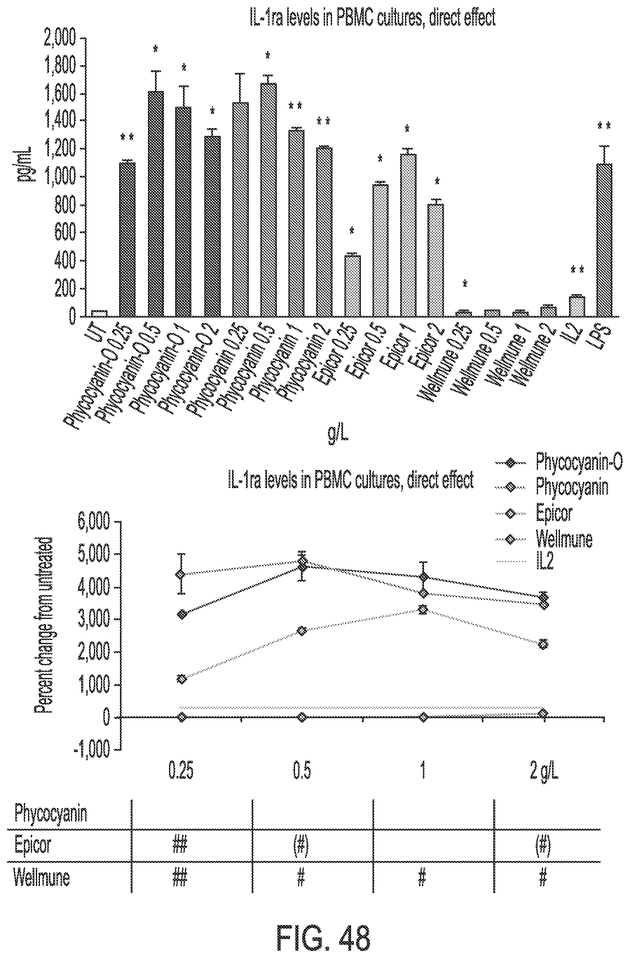

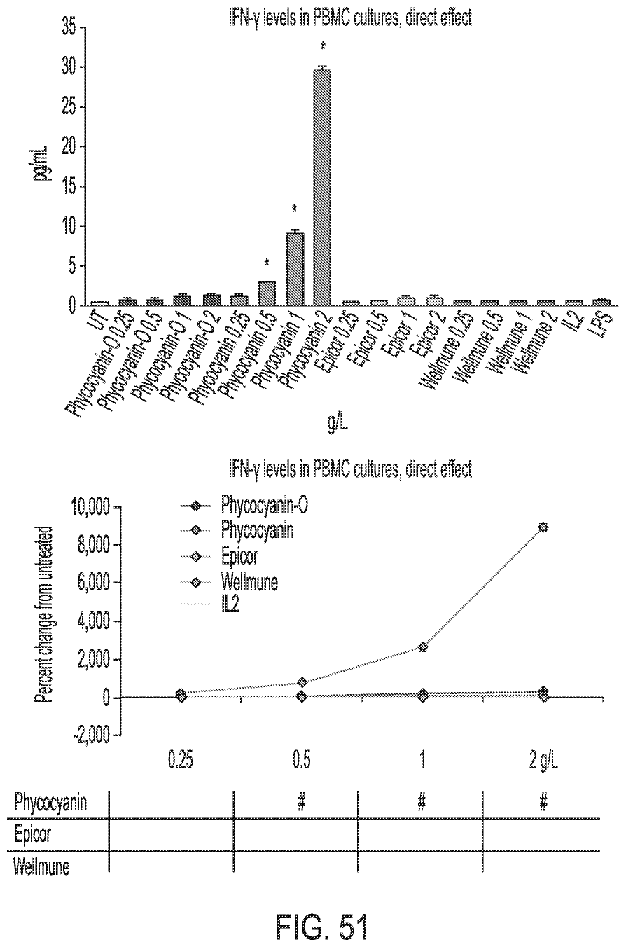

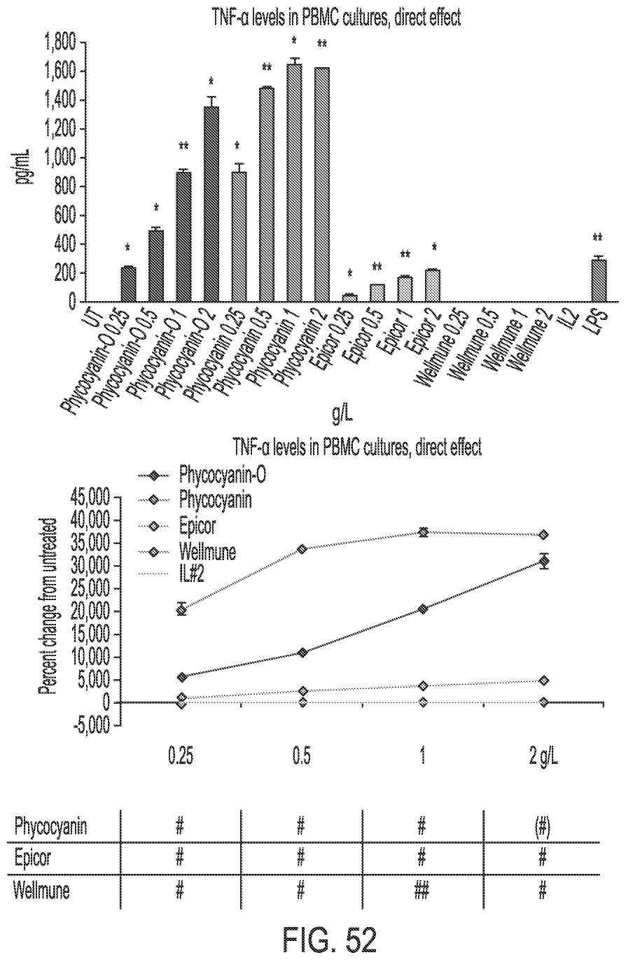

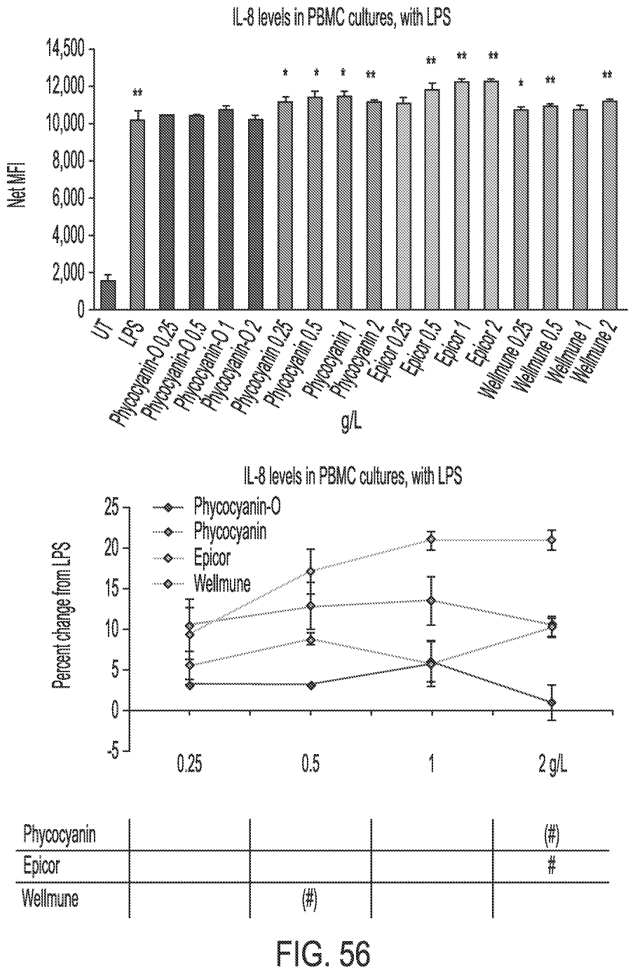

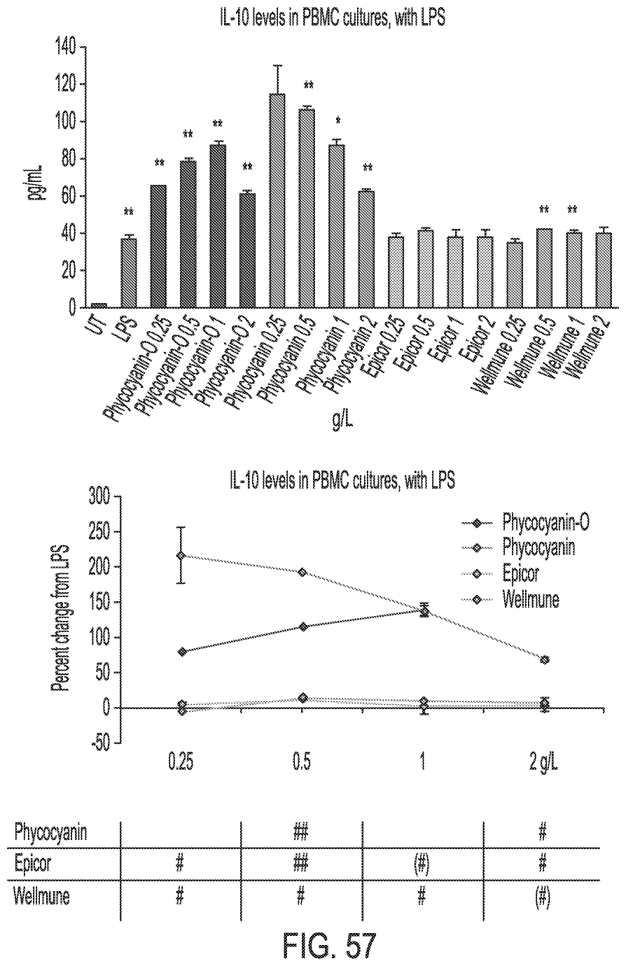

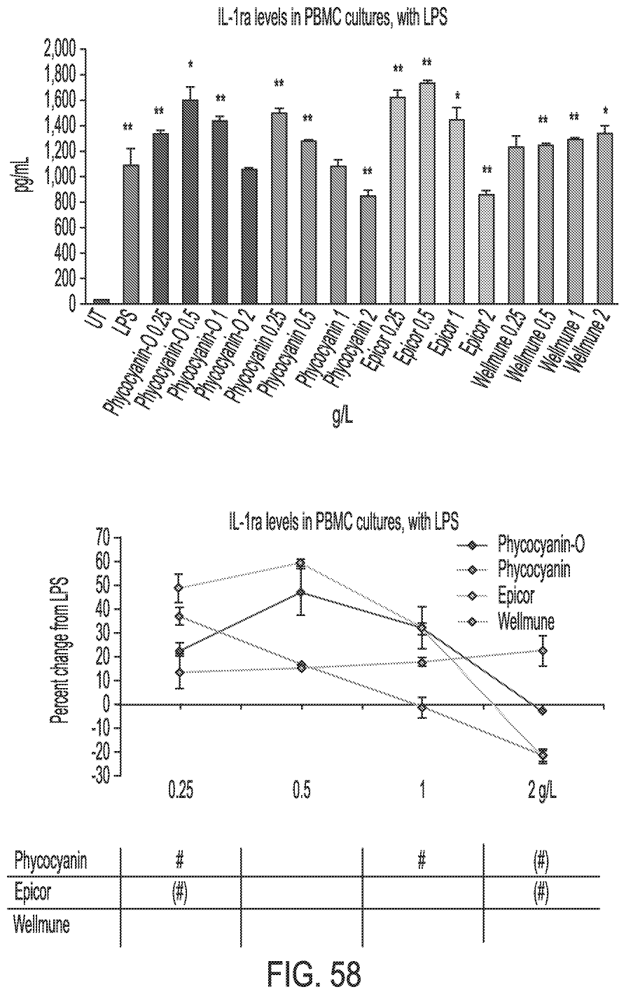

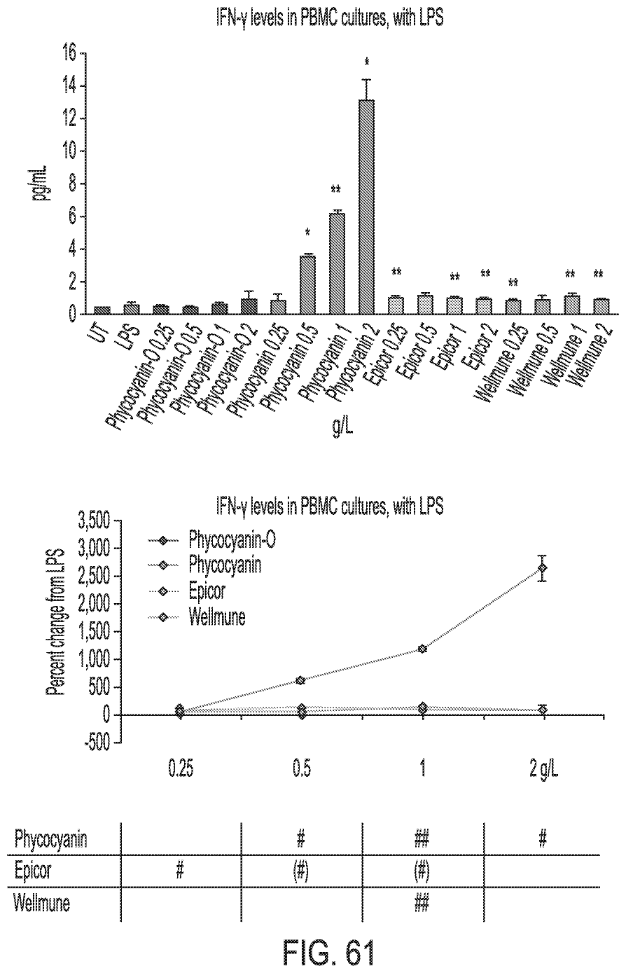

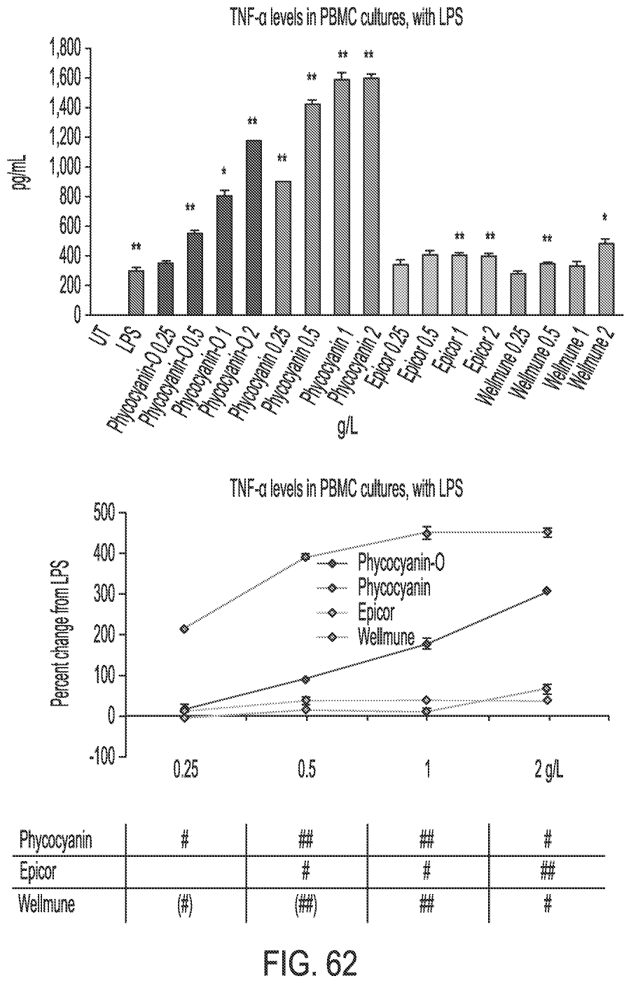

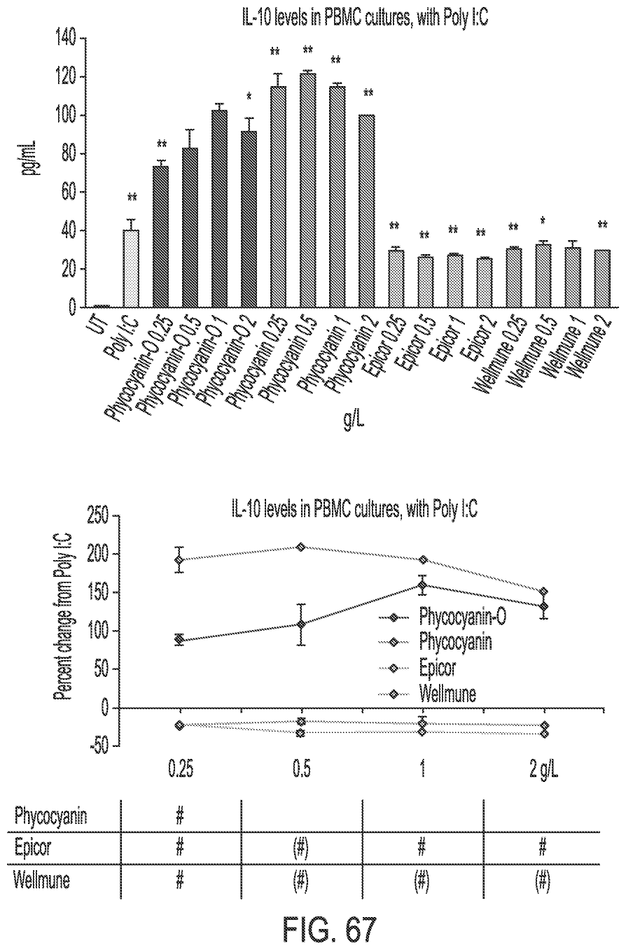

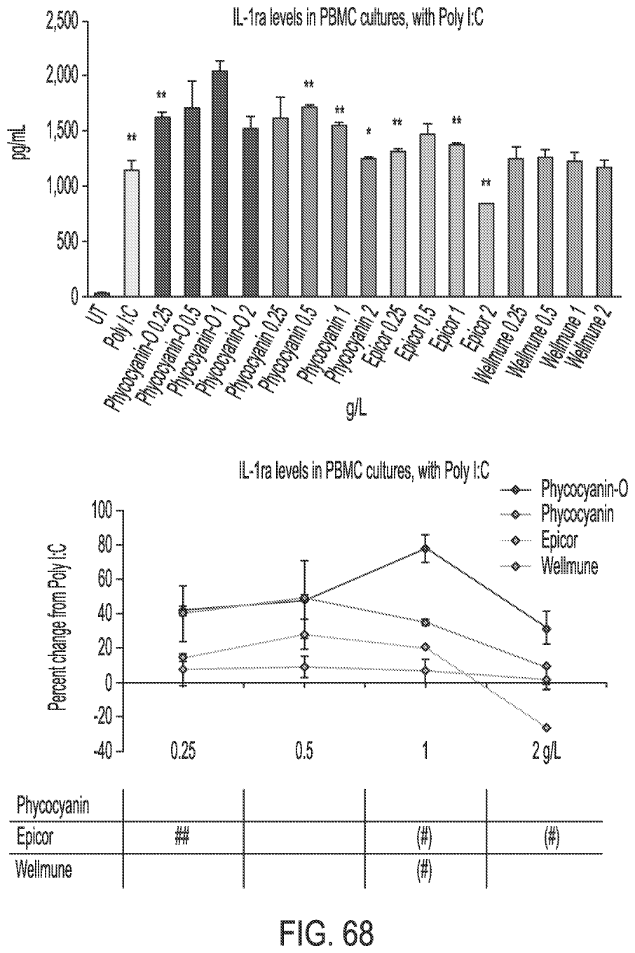

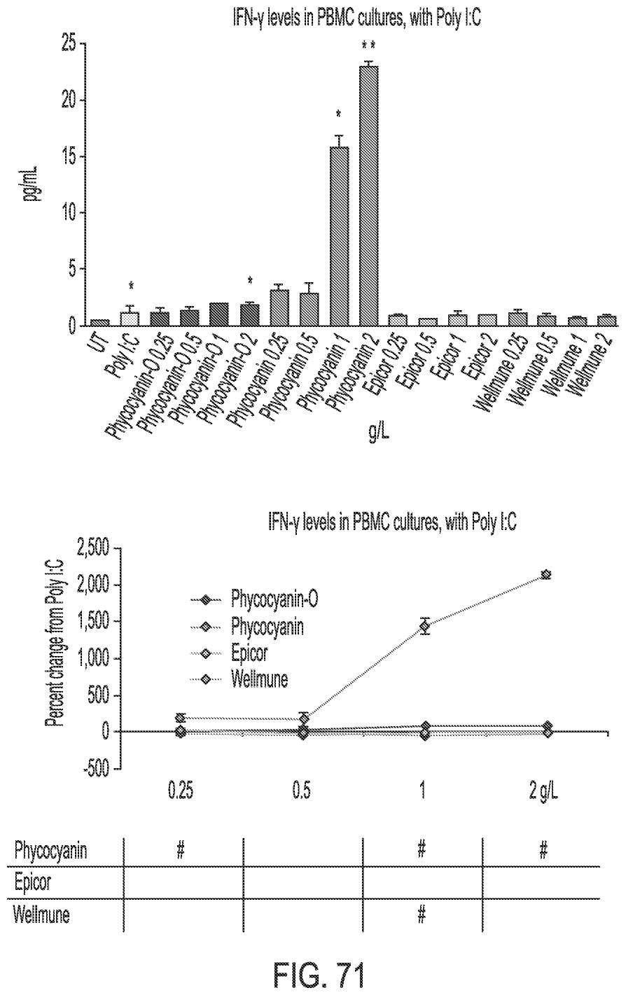

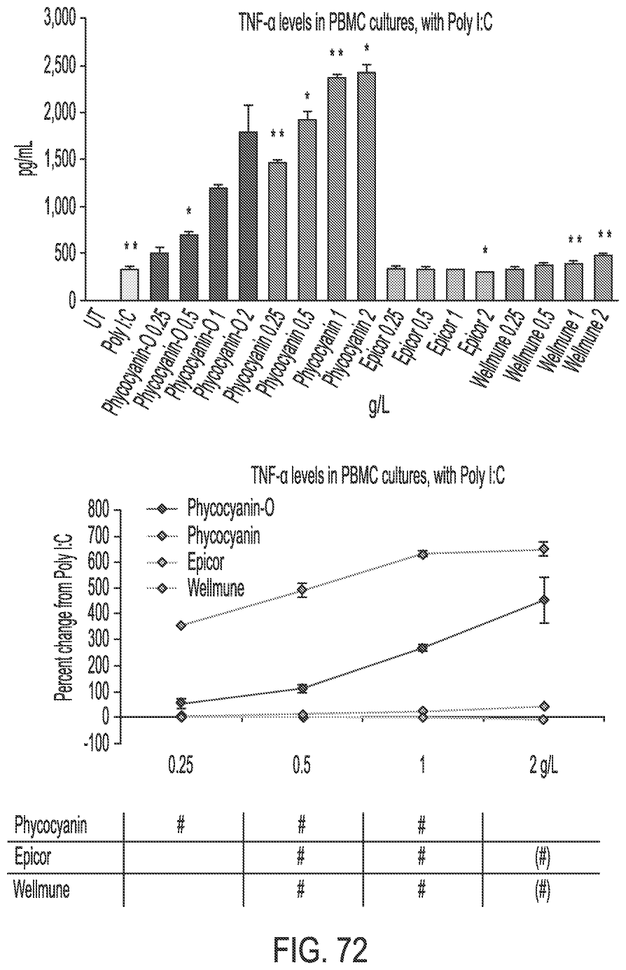

FIGS. 14 to 73 contain asterisks according to the following tables corresponding with the statistical differences compared to the control:

Statistical difference compared to control

| Symbol: | Statistical significance: |
|---|---|
| (*) | P > 0.05 and < 0.1 |
| * | P < 0.05 |
| ** | P < 0.001 |

Statistical difference compared to O-CPs

| Symbol: | Statistical Difference: |
|---|---|
| (#) | P > 0.05 and < 0.1 |
| # | P < 0.05 |
| ## | P < 0.001 |

### DETAILED DESCRIPTION

In the following detailed description, references are made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the spirit or scope of the subject matter presented herein. The aspects of the present disclosure, as generally described herein and illustrated in the Figures, can be arranged, substituted, combined, separated, and designed in a wide variety of different configurations, all of which are contemplated herein and such different configurations would be envisaged by one of skill in the art in view of the instant disclosure.

This disclosure is drawn, inter alia, to certain O-CPs, methods for their production, and treatments utilizing certain chromopeptides and certain O-CPs.

### Methods of Making

Briefly stated, techniques and steps are described herein as methods to produce phycocyanin-derived O-CPs. The methods include filtering a solution of crude PC to separate a protein fraction having a molecular weight more than 10 kDa from compounds having a molecular weight less than or equal to 10 kDa, retaining the protein fraction having a molecular weight more than 10 kDa, hydrolyzing the more than 10 kDa protein fraction with an enzyme to obtain a crude hydrolysate, optionally neutralizing the crude hydrolysate to obtain a neutralized crude hydrolysate, and subjecting the neutralized crude hydrolysate to ultrafiltration to separate the 0-CPs from unhydrolyzed protein, the utilized enzyme and other materials.

The crude PC solution used herein, for example, may comprise 25% pure C-phycocyanin (C-PC), and the remaining 75% comprises natural proteins, peptides, and polysaccharides (phycocyanin alpha and beta subunits, allophycocyanin, phycoerythrin, naturally occurring peptides, amino acids, and sugars), and drying agents or additives (stabilizing agents) to assist in drying (35% trehalose and 5% tri-sodium citrate). The additives as used herein are referred to as low molecular weight complexes or compounds (LMWC) (less than or equal to 10 kDa) and are separated from the large-size protein fraction using a filter. This allows only the larger compounds to remain on the filter, including, for example, C-PC, phycocyanin alpha and beta subunits, allophycocyanin, phycoerythrin, and other peptides. This protein fraction is further digested into O-CPs. Accordingly, this is the unique mixture of O-CPs, which yield the unexpectedly enhanced activity.

The present disclosure demonstrates that hydrolysis of the protein fraction, obtained from ultrafiltration of crude PC having no compounds with a molecular weight of less than or equal to 10 kDa, is much simpler and cost effective for producing O-CPs. Some benefits of the presently disclosed methods are that biocompatible and eco-friendly O-CPs are produced. Further, the chemical hazards associated with production of O-CPs of the prior art, namely methods requiring methanol and strongly acidic conditions (HCl), can be avoided or reduced using methods as disclosed herein. Furthermore, O-CPs made by methods disclosed herein possess unexpectedly enhanced activities, as described further herein.

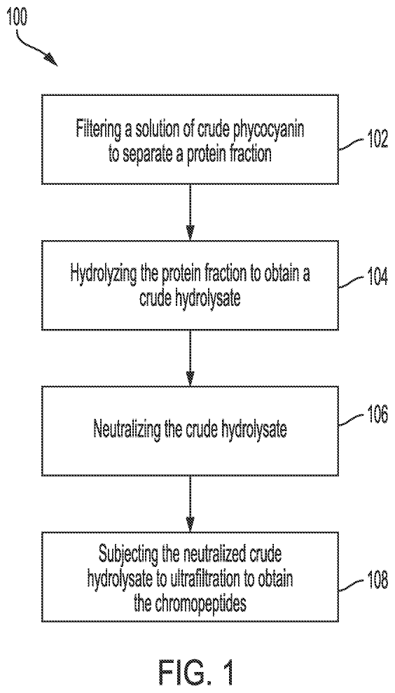

FIG. 1 is a flow chart illustrating a method for producing O-CPs according to one or more embodiments of the present disclosure. The described method 100, filtering a solution of crude PC to separate a protein fraction 102, hydrolyzing the protein fraction to obtain a crude hydrolysate 104, optionally, neutralizing the crude hydrolysate 106, and subjecting the neutralized crude hydrolysate to ultrafiltration to obtain O-CPs 108. At 102, a solution of crude PC is subjected to ultrafiltration to separate a protein fraction. In one example, at 102, the solution of crude PC is processed to remove unwanted LMWCs such as, but not limited to, 0.1 to 10 kDa compounds. In one example, the PC is photometrically characterized before further processing. The ratio of absorbance at 620 nm and 280 nm is used as an indicator of PC purity and the ratio (A₆₂₀/A₂₈₀) of not less than one is maintained throughout the experiments. In one example, crude PC is dissolved in demineralized water to form a solution having a concentration of 50 g/L. In one example, compounds having a molecular weight of less than 10 kDa in size are removed by tangential flow ultrafiltration. The removed low molecular weight compounds are discarded. In one example, the ultrafiltration is carried out at room temperature using a 0.6 m² 10-kDa molecular weight cut-off (MWCO) Hydrosart cassette at 0.1-1 bar pressure and a tangential flow rate of approximately 1-20 L/min, yielding a filtration rate of approximately 0.1-2 L/min. The ultrafiltration is continued until the volume of the retentate. i.e., the protein fraction is reduced to one quarter of the starting volume. The amount of low-molecular-weight compounds is reduced to below 1% of the starting value by repeated washing as calculated by:

$${\%}_{remaining} = 100 \% \times \frac{{V}_{protein fraction}}{{V}_{initial}} \times {( \frac{{V}_{protein fraction}}{{V}_{protein fraction} + {V}_{wash}} )}^{number of washes}$$

The final retentate, i.e., the protein fraction, may either be used directly in the next step at 104, stored frozen at −30° C. (−22° F.), or freeze-dried. The permeate can be discarded as non-hazardous aqueous waste.

After filtering the crude solution 102, the resulting concentration of the protein fraction may then be solubilized, and the concentration of protein fraction in solution may then be adjusted. In some embodiments, the protein fraction may be solubilized in an aqueous solution or water, such as demineralized water. Other aqueous solutions not explicitly listed would readily be envisaged by those of skill in the art, in view of the disclosure contained herein, however certain solvents, such as methanol and other hazardous solvents, would not be contemplated by a skilled artisan nor commensurate in scope with the methods disclosed herein. In certain embodiments, the concentration of protein fraction in solution can be adjusted to about 50 g/L to about 300 g/L, or any range or concentration therebetween.

After filtering and/or adjusting the solution concentration, the solution may then be subjected to hydrolyzing 104. In some embodiments, hydrolyzing 104 may comprise the protein fraction being hydrolyzed using an enzyme. In some embodiments, a protein fraction, as described herein, is combined with an amount of an enzyme. The enzymes include, but are not limited to alcalase, neutrase, protamex, celluclast, viscozyme, and any combination of the foregoing. The amount of an enzyme can be about 0.002 g/g (enzyme to protein) to about 1 g/g. For example, the amount of enzyme may be about 0.002 g/g, 0.01 g/g, 0.05 g/g, 0.1 g/g, 0.2 g/g, 0.3 g/g, 0.4 g/g, 0.5 g/g, 0.6 g/g, 0.7 g/g, 0.8 g/g, 0.9 g/g, 1.0 g/g, or any range or amount in between any two of the preceding values and any other ranges or amounts disclosed herein.

In some embodiments, following the addition of an enzyme, as described herein, the pH, temperature, or both of the solution is adjusted. The adjustment is not particularly limited; however, preferred pH's of the solution are about 6 and about 8, and preferred temperatures of the solution are about 50° C. and about 65° C. Other adjustments, not explicitly listed, would readily be envisaged by those of skill in the art, in view of the disclosure contained herein. Moreover, pH's and temperatures well-outside these ranges, such as strongly acidic (less than pH 2) and other conditions non-compatible with large-scale manufacturing, would not be contemplated by a skilled artisan nor commensurate in scope with the methods disclosed herein.

In certain embodiments, following adjustment of solution pH, temperature, or both, as described herein, the solution is allowed to react. The reaction time may be less than about 1 hr to about 36 hr, or any length of time therebetween. Preferred embodiments within this range are reaction times of about 2 hr, about 5 hr, about 7 hr, and about 24 hr.

Investigative methods were experimentally used to make O-CPs disclosed herein. These methods disclosed herein can use either methanol, HCl, or both, and pepsin. These investigative methods were presented solely as a method of making O-CPs from PC to screen 0-CPs for their activity in vitro or in vivo.

In an alternative method of making O-CPs, the pH of the solution from 102 is adjusted to a value of 1.2 using 32% food-grade hydrochloric acid. This corresponds to a final concentration of approx. 80 mM HCl. A reaction vessel is chosen in such a way that it can withstand low pH values and presence of chlorides. Porcine pepsin is then added to the solution to a final concentration of 1000 FIP units/L (i.e., 0.5 g/L at a standard activity of ≥2,000 FIP units per gram). After initial mixing, agitation or further pH adjustment is not required. The reaction is carried out in a closed vessel to avoid loss of HCl to the atmosphere. The progress of hydrolysis is monitored either by HPLC or photometrically. In one example, the hydrolysis is completed after 36 hours at 37° C. (98.6° F.). The crude hydrolysate is used for the next step at 106. No aqueous waste is generated during this hydrolysis step 104.

Following the hydrolysis reaction 104, the crude hydrolysate is neutralized 106 to return to composition to a neutral pH (pH 7). In some instances, neutralizing the pH of the crude hydrolysate 106 is performed using food-grade NaOH or HCl solution; the compound used would be readily understood in the context of the pH used to perform hydrolysis 104. While performing neutralizing, it is essential to avoid over titration, as negative effect on the product stability can be seen if the pH is too low or too high.

Following either the hydrolysis reaction 104 or the neutralizing step 106, the resulting product may optionally be subjected to filtration, such as tangential flow ultrafiltration (10 kDa MWCO) 108, to separate the desired O-CPs of less than 10 kDa in the permeate from the unwanted insoluble contaminants, enzyme, unhydrolyzed protein, and the like. The enzyme can include unreacted enzyme or spent enzyme that has been utilized for hydrolyzing the protein fraction. In certain embodiments, filtration 108 can be carried out at room temperature using a 0.6 m² 10-kDa MWCO Hydrosart (SARTORIUS) cassette at 0.1-1 bar pressure and a tangential flow rate of about 1 L/min to about 20 L/min, or a filtration rate of about 0.1 L/min to about 2 L/min. In some embodiments, the filtration may be continued until the volume of the retentate is reduced as much as possible. The instances in which filtration 108 is performed on the product of the hydrolysis reaction 104 product or the product of the neutralizing step 106 would readily be understood in context. For example, a method utilizing no HCl during the hydrolysis reaction 104 may not need to be subject to the neutralizing step, and therefore were filtration 108 to be performed, it may be performed on the product following the hydrolysis reaction 104. Similarly, if the hydrolysis reaction 104 were performed in strongly acidic conditions (HCl), the product of the hydrolysis reaction 104 could be subjected to a neutralizing step 106, and therefore were filtration 108 to be performed, it could be performed on the product following the neutralizing step 106.

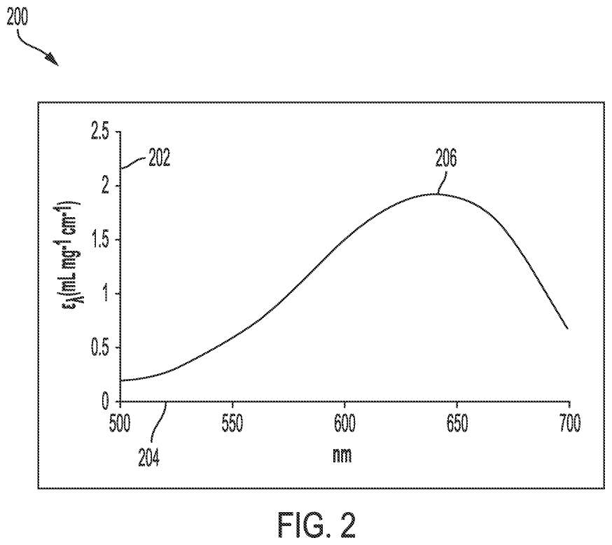

FIG. 2 is a graph 200 illustrating the absorption spectrum between 500 and 700 nm of a complete digest of PC using pepsin in 1 M HCl for 24 hr. The vertical axis 202 represents an extinction coefficient and the horizontal axis 204 represents the absorbance. The absorbance curve 206 indicates the variation of the extinction coefficient with absorbance. The mass extinction coefficient for O-CPs is about 10 fold greater and peaks at about 640 nm, with an extinction coefficient of 1.92 mL mg⁻¹ cm⁻¹. Compared with PC at neutral pH, the peak is red-shifted, and the extinction coefficient is less than half, as the O-CPs yield from PC is only about 10.2%, the extinction coefficient expressed for O-CPs is 18.8 mL mg⁻¹ cm⁻¹.

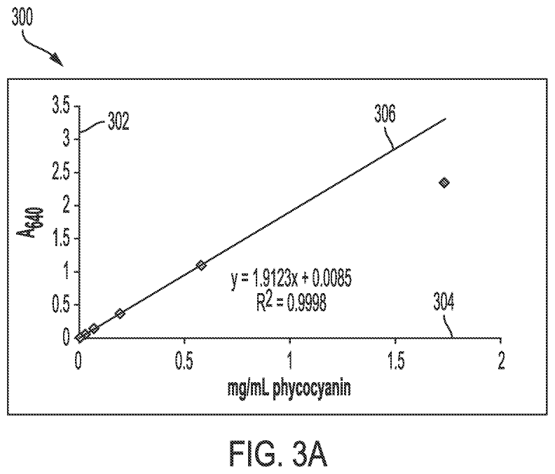

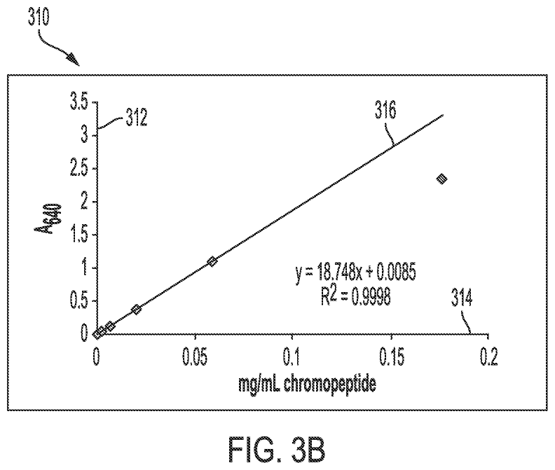

FIG. 3A is a graph 300 illustrating the calibration curve for photometric determination of PC, whereas FIG. 3B is a graph 310 illustrating the calibration curve for photometric determination of PC expressed in terms of O-CPs content. In FIG. 3A, the vertical axis 302 represents absorbance at 640 nm and the horizontal axis 304 represents the concentration of PC. The best-fit line 306 indicates a linearity for the standard curve up to at least about 0.6 mg/mL of PC. In FIG. 3B, the vertical axis 312 represents absorbance at 640 nm and the horizontal axis 314 represents the concentration of PC. The best-fit line 316 indicates a linearity for the standard curve up to at least about 0.06 mg/mL of O-CPs. Quantification is assumed to be reliable for A₆₄₀ values between 0.05 and 1. The slopes of the lines correspond well with the extinction coefficients determined, with 1.92 mL mg⁻¹ cm⁻¹ versus 1.91 mL mg⁻¹ cm⁻¹ for the test standard for PC and 18.8 mL mg⁻¹ cm⁻¹ versus 18.7 mL mg⁻¹ cm⁻¹ for O-CPs produced by the methods disclosed herein.

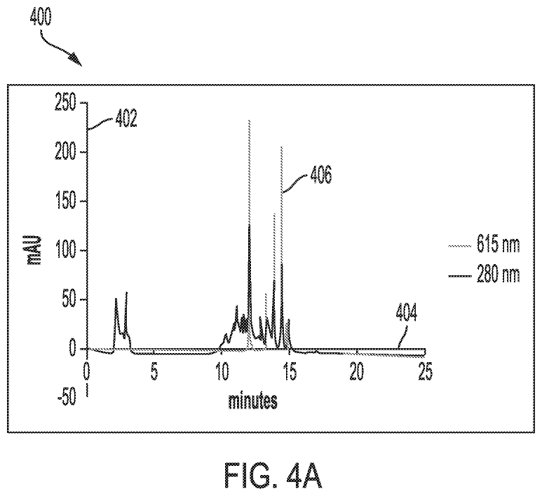

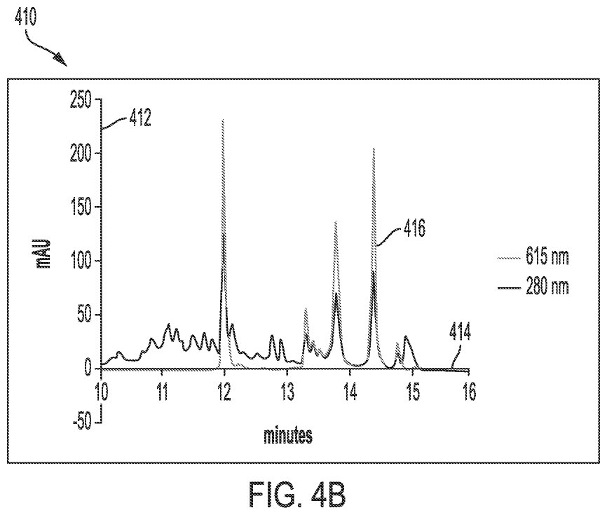

In addition to photometric characterization as shown in FIGS. 3A and 3B, a protocol for the quantification of PC by HPLC is also established. HPLC gives the added advantage of giving an indication of the number of O-CPs obtained by complete digest. FIG. 4A is a graph 400 illustrating complete chromatograms, that contain O-CPs, detected at 280 nm and 615 nm over a 25 minute run-time, whereas FIG. 4B is a graph 410 illustrating chromatograms, that contain 0-CPs, detected at 280 nm and 615 nm during in the 10-16 minute range. In FIG. 4A, the vertical axis 402 represents the measure of the intensity of absorbance in milli-Absorbance units (mAU) and the horizontal axis 404 represents time in minutes. The chromatogram 406 indicates the retention time for each peak. Similarly, in FIG. 4B, the vertical axis 412 represents the measure of the intensity of absorbance in mAU and the horizontal axis 414 represents time in minutes. The chromatogram 416 indicates the retention time for each peak. Detection at 280 nm is suitable for all peptides containing aromatic residues, particularly tryptophan, while detection at 615 nm is specific to O-CPs. No peaks at 615 nm were detected outside of the range between 10 and 16 minutes.

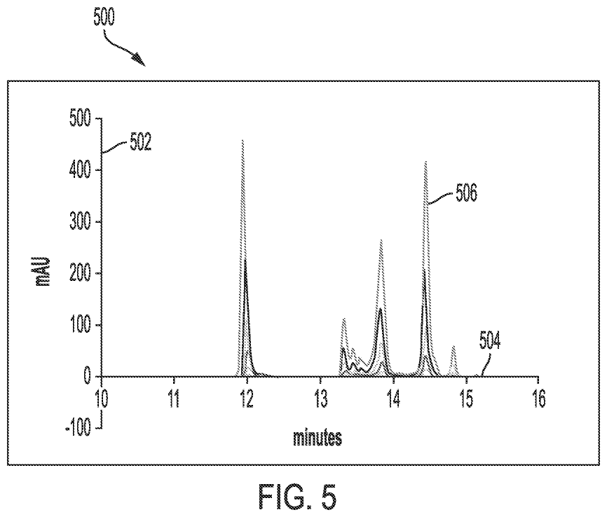

FIG. 5 is a graph 500 illustrating HPLC chromatographs for complete digests of PC based on absorption at 615 nm. In FIG. 5, the vertical axis 502 represents the measure of the intensity of absorbance in mAU and the horizontal axis 504 represents time in minutes. The chromatograms 506 indicates the retention time for each of the major and minor peaks. Five major and two minor peaks are detected.

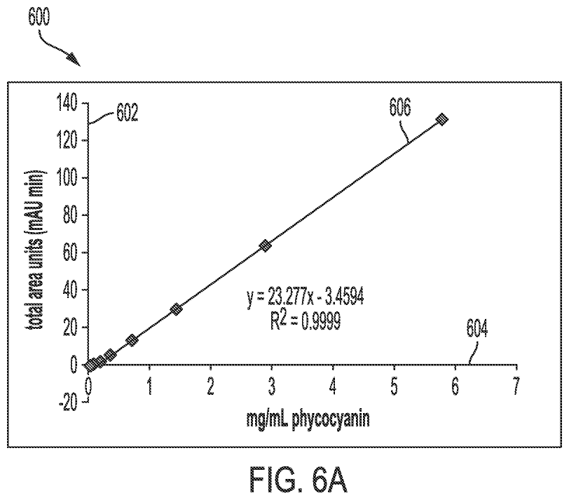

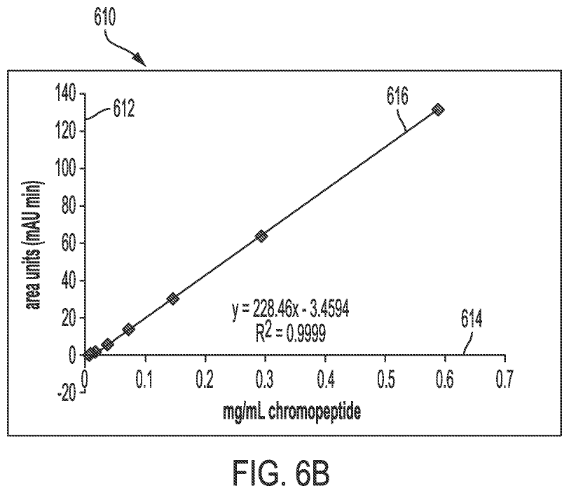

FIG. 6A is a graph 600 illustrating the calibration curve for the quantification of O-CPs by HPLC expressed in terms of PC. In FIG. 6A, the vertical axis 602 represents total area units (mAU min) and the horizontal axis 604 represents concentration of PC (mg/mL). The calibration curve 606 indicates a correlation between total area and concentration was highly linear within over the entire concentration range, and is captured by the equation:

y=23.277x−3.4594 with an R² of 0.9999

FIG. 6B is a graph 610 illustrating the calibration curve for the quantification of O-CPs by HPLC expressed in terms of O-CPs. In FIG. 6B, the vertical axis 612 represents total area units (mAU min) and the horizontal axis 614 represents concentration of chromopeptides (mg/mL). The calibration curve 616 indicates a correlation between total area and concentration was highly linear within over the entire concentration range, and is captured by the equation:

y=228.46x−3.4594 with an R² of 0.9999

### Methods of Use

Briefly stated, compositions are generally described treating, preventing, or ameliorating age-related somatic disease, bacterial and/or viral infections/diseases, and diseases associated with oxidative stress, or symptoms associated with any of the foregoing. The compositions show unexpected results that the O-CPs provide unexpected and improved properties when compared to either PC or commercially available immune modulating supplements such as, but not limited to, EpiCor and Wellmune, as well as various compounds or forms of zinc such as, but not limited to, zinc gluconate and zinc picolinate. The O-CPs provide unexpected and improved properties in terms of total antioxidant capacity, cellular antioxidant protection or bioavailability, and immune activation and cytokine production in various instances such as, but not limited to, under 3 culture conditions, namely, direct effects (unstressed cultures), effects in context of a bacterial inflammatory immune challenge, and effects in context of a viral mimetic immune challenge. Accordingly, described herein are compositions that provide for effective activity of O-CPs, and methods of treatment thereof, that include O-CPs alone or in combination with other compounds to provide immunomodulatory effects. In some embodiments compositions disclosed herein may be administered with nicotinamide riboside (NR), selenium or a compound thereof, such as selenomethionine, zinc or a compound thereof, such as zinc picolinate, or combinations thereof. In some instances, compositions comprising O-CPs and NR show unexpected results in that the combination provides unexpected and improved properties when compared to either O-CPs or NR alone. The compositions also show unexpected results in that the O-CPs provide unexpected and improved properties when compared to conventional dietary supplements. Similarly, the combination of O-CPs with zinc picolinate and selenomethionine provides unexpected and improved properties when compared to either O-CPs, zinc picolinate, or selenomethionine alone. Accordingly, described herein are compositions comprising an effective dose that provide for therapeutic or nutraceutical activity to a subject, uses and methods of treatment thereof, that include a therapeutically effective amount of O-CPs and at least one of the NR, zinc picolinate, selenomethionine, or any combination thereof.

As disclosed herein, immunomodulatory effects or immunomodulation refers to immune activation and modulation or immunosuppression in context of bacterial and viral challenges or infections. Immune activation means that effector cells of the immune system are activated in order to proliferate, migrate, differentiate, or become active in any other form. On the other hand, immunosuppression or modulation refers as reducing the activation or efficacy of the immune system. Immunomodulation, in this context, can also be referred as influencing the nature or the character of an immune reaction, either by affecting an immune reaction, which is still developing or maturing, or by modulating the character of an established immune reaction.

In some embodiments, O-CPs compositions can comprise a therapeutically effective amount of O-CPs and at least one pharmaceutically acceptable excipient for treating, preventing or ameliorating, or preventing age-related somatic diseases or bacterial or viral infections. In embodiments, the composition can comprise a therapeutically effective amount of O-CPs in combination with NR for treating, preventing or ameliorating, or preventing age-related somatic diseases in a subject. In some embodiments, the composition can comprise a therapeutically effective amount of O-CPs in combination with zinc picolinate and/or selenomethionine for treating, preventing or ameliorating, or preventing bacterial or viral infections in a subject.

As used herein, the expression “therapeutically effective amount” includes a non-toxic but sufficient amount of a composition for use in the embodiments disclosed herein to provide the desired therapeutic or nutraceutical effect. The amount of the active ingredient of the composition disclosed herein can vary in different subjects depending on various factors such as, but not limited to, the species being treated, age and general condition of the subject, severity of the condition being treated, particular active ingredient being administered, weight of the subject, and the mode of administration. Thus, to specify an exact “effective amount” can vary based on the embodiments disclosed herein. Moreover, a suitable “effective amount” can be determined by a person skilled in the art for a particular case in view of the disclosure contained herein.

By way of example, a “therapeutically effective amount” of the O-CPs disclosed herein, for treatment of age-related somatic diseases can be, for example (in dosage per weight of the subject), 30 mg/70 kg or 0.428 mg/kg, 300 mg/70 kg or 4.286 mg/kg, 3 g/70 kg or 0.0428 g/kg, or any fraction in between this range of the O-CPs for a human subject. Accordingly, in some embodiments, the dose of O-CPs in the compositions disclosed herein can be about 0.428 mg/kg per day, 4.286 mg/kg per day or 0.0428 g/kg per day for the human subject. A conversion factor of, e.g., 6.17 can be used to convert human doses to rat or murine doses. Accordingly, in some embodiments, a “therapeutically effective amount” of the O-CPs disclosed herein for treatment of age-related somatic diseases can be, for example, 2.64 mg/kg, 26.44 mg/kg, 0.264 g/kg, or any fraction in between this range of the O-CPs for a murine subject. Accordingly, in some embodiments, the dose of O-CPs in the compositions disclosed herein can be about 2.64 mg/kg per day, 26.44 mg/kg per day, or 0.264 g/kg per day, for the murine subject. The exemplary therapeutically effective amounts listed above can, in some embodiments, be administered on a daily basis, e.g., for 21 consecutive days, as required to achieve the desired therapeutic effect in the subject.

By way of example, a “therapeutically effective amount” of the NR (used in combination with O-CPs) as disclosed herein, for treatment of age-related somatic diseases can be, for example, 30 mg/70 kg to 300 mg/70 kg or 0.428 mg/kg to 4.286 mg/kg, for a human subject. Accordingly, in some embodiments, the dose of NR in the compositions disclosed herein can be about 0.428 mg/kg to about 4.286 mg/kg per day for the human subject. A conversion factor of, e.g., 6.17 can be used to convert human doses to rat or murine doses. Accordingly, in some embodiments, a “therapeutically effective amount” of the NR disclosed herein, for treatment of age-related somatic diseases can be, for example, 2.64 mg/kg, or 26.44 mg/kg, for a murine subject. Accordingly, in some embodiments, the dose of NR in the compositions disclosed herein can be about 2.64 mg/kg per day to about 26.44 mg/kg per day for the murine subject. The exemplary therapeutically effective amounts listed above can, in some embodiments, be administered on a daily basis, e.g., for 21 consecutive days, as required to achieve the desired therapeutic effect in the subject.

By way of example, a “therapeutically effective amount” of the O-CPs disclosed herein, for treatment of bacterial or viral infections can be, for example, 30 mg/70 kg to 3 g/70 kg or 0.428 mg/kg to 0.0428 g/kg for a human subject. Accordingly, in some embodiments, the dose of O-CPs in the compositions disclosed herein can be about 0.428 mg/kg to about 0.0428 g/kg per day for the human subject. A conversion factor of, e.g., 12.33 can be used to convert human doses to mouse or murine doses. Accordingly, in some embodiments, a “therapeutically effective amount” of the O-CPs disclosed herein for treatment of bacterial or viral infections can be, for example, 5.28 mg/kg or 0.528 g/kg for a murine subject. Accordingly, in some embodiments, the dose of O-CPs in the compositions disclosed herein can be about 5.28 mg/kg per day to about 0.528 g/kg per day for the murine subject. The exemplary therapeutically effective amounts listed above can, in some embodiments, be administered to the subject, e.g., 2 weeks before the LPS treatment, as required to achieve the desired therapeutic effect in the subject.

By way of example, a “therapeutically effective amount” of the zinc picolinate disclosed herein, for treatment of bacterial or viral infections can be, for example, 5 mg/70 kg to 100 mg/70 kg or 0.071 mg/kg to 1.428 mg/kg for a human subject. Accordingly, in some embodiments, the dose of zinc picolinate in the compositions disclosed herein can be about 0.071 mg/kg per day to about 1.428 mg/kg per day for the human subject. A conversion factor of, e.g., 12.33 is used to convert human doses to mouse or murine doses. Accordingly, in some embodiments, a “therapeutically effective amount” of the zinc picolinate disclosed herein for treatment of bacterial or viral infections can be, for example, 0.875 (elemental zinc) mg/kg to 17.61 (elemental zinc) mg/kg, or 4.375 mg/kg to 88.05 mg/kg for a murine subject. Accordingly, in some embodiments, the dose of zinc picolinate in the compositions disclosed herein can be about 0.875 (elemental zinc) mg/kg per day to about 17.61 (elemental zinc) mg/kg per day, or 4.375 mg/kg per day to 88.05 mg/kg per day, for the murine subject. The exemplary therapeutically effective amounts listed above can, in some embodiments, be administered to the subject, e.g., 2 weeks before the LPS treatment, as required to achieve the desired therapeutic effect in the subject.

By way of example, a “therapeutically effective amount” of the selenomethionine disclosed herein, for treatment of bacterial or viral infections can be, for example, 50 μg/70 kg to 500 μg/70 kg or 0.714 μg/kg to 7.14 μg/kg for a human subject. Accordingly, in some embodiments, the dose of selenomethionine in the compositions disclosed herein can be about 0.714 μg/kg per day to about 7.14 μg/kg per day for the human subject. A conversion factor of, e.g., 12.33 is used to convert human doses to mouse or murine doses. Accordingly, in some embodiments, a “therapeutically effective amount” of the selenomethionine disclosed herein for treatment of bacterial or viral infections can be, for example, 8.80 μg/kg or 88.0 μg/kg for a murine subject. Accordingly, in some embodiments, the dose of selenomethionine in the compositions disclosed herein can be about 8.80 μg/kg per day to about 88.0 μg/kg per day for the murine subject. The exemplary therapeutically effective amounts listed above can, in some embodiments, be administered to the subject, e.g., 2 weeks before the LPS treatment, as required to achieve the desired therapeutic effect in the subject.

In some embodiments, O-CPs compositions, as described herein, may further comprise an amount of enzyme. In certain embodiments, said enzyme can be the enzyme used in a hydrolysis reaction to prepare the O-CPs from PC. The enzyme, if contained, is therefore preferably of a quality that is at least “generally recognized as safe” (GRAS), and more preferably the enzyme is therefore at least “food grade” enzyme. In certain embodiments, the enzyme may be alcalase. The amount of enzyme present is not particularly limited, and may be about 10 μg to about 10 g. For example, the amount of enzyme the composition can be 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 125 μg, 150 μg, 175 μg, 200 μg, 225 μg, 250 μg, 275 μg, 300 μg, 325 μg, 350 μg, 375 μg, 400 μg, 425 μg, 450 μg, 475 μg, 500 μg, 525 μg, 575 μg, 600 μg, 625 μg, 650 μg, 675 μg, 700 μg, 725 μg, 750 μg, 775 μg, 800 μg, 825 μg, 850 μg, 875 μg, 900 μg, 925 μg, 950 μg, 975 μg, 1000 μg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1.25 g, 1.5 g, 1.75 g, 2.0 g, 2.25 g, 2.5 g, 2.75 g, 3.0 g, 3.25 g, 3.5 g, 3.5 g, 3.75 g, 4.0 g, 4.25 g, 4.5 g, 4.75 g, 5.0 g, 5.25 g, 5.5 g, 5.75 g, 6.0 g, 6.25 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, or more, or any range or amount in between any two of the preceding values and any other ranges or amounts disclosed herein.

Some embodiments of the compositions disclosed herein, may further comprise at least one pharmaceutically acceptable excipient, or vehicle. For example, pharmaceutically acceptable vehicles can include carriers, excipients, diluents, and the like, as well as combinations or mixtures thereof. As used herein, an “excipient” refers to a substance that is added to a composition for the purpose of long-term stabilization, bulk, consistency, binding ability, lubrication, disintegrating ability, drug absorption, enhanced solubility etc. A “diluent” is a type of excipient.

As used herein, a “carrier” refers to a compound that interacts with and enhances the properties of active ingredients or which facilitates the incorporation of a compound into cells or tissues of a subject.

As used herein, a “diluents” refers to a constituent that acts as filler in a formulation to increase weight and improve content uniformity. The diluent may be necessary or desirable in a formulation.

In some embodiments, the compositions disclosed herein are administered through various delivery modes including, but not limited to, orally, intravenously, or subcutaneously. Other modes of administration of the compositions include, but are not limited to, intradermal, intramuscular, or intraperitoneal. The compositions described herein can be administered to human subjects. In some embodiments, the compositions are mixed with other active ingredients, or carriers, diluents, excipients, or combinations thereof.

In some embodiments, compositions, as disclosed herein, can take the form of solutions, suspensions, emulsion, tablets, pills, pellets, capsules, capsules containing liquids, powders, sustained-release formulations, suppositories, emulsions, aerosols, sprays, chewable dosage forms, gelatin dosage forms, liquid dosage forms, or any other form suitable for use.

The compositions as disclosed herein can be provided in a blister pack or dispenser device or the like. The blister pack can include, for example, metal or plastic foil or the like. The dispenser device can be accompanied by instructions for administration of the compositions. The compositions as disclosed herein can be provided in a suitable container and labeled for treatment of an indicated condition or disease.

In some embodiments, compositions, as described herein, can be administered to human subjects. In some embodiments, the compositions are mixed with other active ingredients, or carriers, diluents, excipients, or combinations thereof. Compositions intended for oral use can be prepared according to any method known in the art for the manufacture of pharmaceutically acceptable compositions and such compositions may include one or more of the following agents: sweeteners, flavoring agents, coloring agents, coatings, and preservatives. The sweetening and flavoring agents will increase the palatability of the preparation. Tablets containing the complexes in admixture with non-toxic pharmaceutically acceptable excipients suitable for tablet manufacture are acceptable. Pharmaceutically acceptable vehicles such as excipients are compatible with the other ingredients of the formulation (as well as non-injurious to the patient). Such excipients include inert diluents such as calcium carbonate, sodium carbonate, lactose, calcium phosphate or sodium phosphate; granulating and disintegrating agents, such as corn starch or alginic acid; binding agents such as starch, gelatin, or acacia; and lubricating agents such as magnesium stearate, stearic acid, or talc. Tablets can be uncoated or can be coated by known techniques to delay disintegration and absorption in the gastrointestinal tract and thereby provide a sustained action over a longer period of time. For example, a time delay material such as glyceryl monostearate or glyceryl distearate alone or with a wax can be employed.

Formulations for oral use can also be presented as hard gelatin-containing or non-gelatinous capsules wherein the active ingredient is mixed with an inert solid diluent, for example calcium carbonate, calcium phosphate or kaolin, or as soft gelatin capsules wherein the active ingredient is mixed with water or an oil medium, such as peanut oil, liquid paraffin, or olive oil. Aqueous suspensions can contain the complex of the described herein admixed with excipients suitable for the manufacture of aqueous suspensions. Such excipients include suspending agents, dispersing, or wetting agents, one or more preservatives, one or more coloring agents, one or more flavoring agents and one or more sweetening agents such as sucrose or saccharin.

Oil suspensions can be formulated by suspending the active ingredient in a vegetable oil, such as arachis oil, olive oil, sesame oil or coconut oil, or in a mineral oil such as liquid paraffin. The oil suspension can contain a thickening agent, such as beeswax, hard paraffin or cetyl alcohol. Sweetening agents, such as those set forth above, and flavoring agents can be added to provide a palatable oral preparation. These compositions can be preserved by an added antioxidant such as ascorbic acid. Dispersible powders and granules suitable for preparation of an aqueous suspension by the addition of water can provide the active ingredient in admixture with a dispersing or wetting agent, a suspending agent, and one or more preservatives. Additional excipients, for example sweetening, flavoring, and coloring agents, can also be present.

Syrups and elixirs can be formulated with sweetening agents, such as glycerol, sorbitol, or sucrose. Such formulations can also contain a demulcent, a preservative, a flavoring, or a coloring agent.

The composition for parenteral administration can be in the form of a sterile injectable preparation, such as a sterile injectable aqueous or oleaginous suspension. This suspension can be formulated according to methods well known in the art using suitable dispersing or wetting agents and suspending agents. The sterile injectable preparation can also be a sterile injectable solution or suspension in a non-toxic parenterally-acceptable diluent or solvent, such as a solution in 1,3-butanediol. Suitable diluents include, for example, water, Ringer's solution, and isotonic sodium chloride solution. In addition, sterile fixed oils can be employed conventionally as a solvent or suspending medium. For this purpose, any bland fixed oil can be employed including synthetic mono or diglycerides. In addition, fatty acids such as oleic acid can likewise be used in the preparation of injectable preparations.

It will be appreciated that the amount of the compound may be combined with a carrier material to produce a single dosage form. Such forms will vary depending upon the host treated and the particular mode of administration.

In some embodiments, compositions described herein may be administered via supplements or dosages designed for animals. In some animal applications, the compound or composition may be added to and/or comprise a pet treat or biscuit, for example, a dog biscuit or a cat treat.

Aqueous suspensions may contain the compound disclosed herein in admixture with excipients suitable for the manufacture of aqueous suspensions. Such excipients include suspending agents, dispersing, or wetting agents, one or more preservatives, one or more coloring agents, one or more flavoring agents and one or more sweetening agents such as sucrose or saccharin.

The compositions intended for oral administration can be prepared according to any method known in the art for manufacturing the compositions, and such compositions may also contain at least one of the agents, namely, sweeteners, flavoring agents, coloring agents, and preservatives.

In some embodiments, the compositions disclosed herein may be formulated in a controlled release vehicle. Utilization of controlled release vehicles would readily be envisaged by those of skill in the pharmaceutical sciences in view of the disclosure contained herein, and these aspects can be applied to nutritional and dietary supplements. The technology and products in this art are variably referred to as controlled release, sustained release, prolonged action, depot, repository, delayed action, retarded release, and timed release; the words “controlled release” as used herein is intended to incorporate each of the foregoing technologies.

Numerous controlled release vehicles can be used, including biodegradable or bioerodable polymers such as polylactic acid, polyglycolic acid, and regenerated collagen. Controlled release drug delivery devices can include creams, lotions, tablets, capsules, gels, microspheres, liposomes, ocular inserts, minipumps, and other infusion devices such as pumps and syringes. Implantable or injectable polymer matrices, and transdermal formulations, from which active ingredients are slowly released, and can be used in the disclosed methods.

Controlled release preparations can be achieved by the use of polymers to form complexes with or absorb a composition. The controlled delivery can be exercised by selecting appropriate macromolecules such as polyesters, polyamino acids, polyvinylpyrrolidone, ethylenevinyl acetate, methylcellulose, carboxymethylcellulose, and protamine sulfate, and the concentration of these macromolecule as well as the methods of incorporation are selected in order to control release of active complex.

Controlled release of active complexes can be taken to mean any of the extended release dosage forms. The following terms may be considered to be substantially equivalent to controlled release, for the purposes of the present disclosure: continuous release, controlled release, delayed release, depot, gradual release, long term release, programmed release, prolonged release, programmed release, proportionate release, protracted release, repository, retard, slow release, spaced release, sustained release, time coat, time release, delayed action, extended action, layered time action, long acting, prolonged action, sustained action medications and extended release, release in terms of pH level in the gut and intestine, breakdown of the molecule and based on the absorption and bioavailability.

Hydrogels, wherein a composition as disclosed herein is dissolved in an aqueous constituent to gradually release over time, can be prepared by copolymerization of hydrophilic mono-olefinic monomers such as ethylene glycol methacrylate. Matrix devices, wherein a composition is dispersed in a matrix of carrier material, can be used. The carrier can be porous, non-porous, solid, semi-solid, permeable or impermeable. Alternatively, a device comprising a central reservoir of a composition disclosed herein surrounded by a rate controlling membrane can be used to control the release of the complex. Rate controlling membranes include ethylene-vinyl acetate copolymer or butylene terephthalate/polytetramethylene ether terephthalate. Use of silicon rubber or ethylene-vinyl alcohol depots are also contemplated.

Controlled release oral formulations can also be used. In an embodiment, a composition as described herein is incorporated into a soluble or erodible matrix, such as a pill or a lozenge. In another example, the oral formulations can be a liquid used for sublingual administration. These liquid compositions can also be in the form a gel or a paste. Hydrophilic gums, such as hydroxymethylcellulose, are commonly used. A lubricating agent such as magnesium stearate, stearic acid, or calcium stearate can be used to aid in the tableting process.

The compositions described herein may be administered once, twice, or three times per day. In some embodiments, the compositions are administered four times a day. For example, the compositions may be administered before, after, or during a meal. Dosing for oral administration may be with a regimen calling for single daily dose, or for a single dose every other day, or for a single dose within 72 hours of the first administered dose, or for multiple, spaced doses throughout the day. The active agents which make up the therapy may be administered simultaneously, either in a combined dosage form or in separate dosage forms intended for substantially simultaneous oral administration. The active agents which make up the therapy may also be administered sequentially, with either active component being administered by a regimen calling for two-step ingestion. Thus, a regimen may call for sequential administration of the active agents with spaced-apart ingestion of the separate, active agents. The time period between the multiple ingestion steps may range from a few minutes to as long as about 72 hours, depending upon the properties of each active agent such as potency, solubility, bioavailability, plasma half-life and kinetic profile of the agent, as well as depending upon the age and condition of the patient. The active agents of the therapy whether administered simultaneously, substantially simultaneously, or sequentially, may involve a regimen calling for administration of one active agent by oral route and the other active agent by intravenous route. In one aspect, the embodiments described herein can achieve therapeutic and/or nutraceutical benefits not previously recognized or achievable, and thus, unexpectedly and surprisingly achieve improved abilities for using the compositions. In some embodiments a composition is formulated for intravenous administration because a more concentrated solution can be produced. Whether the active agents of the therapy are administered by oral or intravenous route, separately or together, each such active agent will be contained in a suitable pharmaceutical formulation of pharmaceutically-acceptable excipients, diluents, or other formulations components.

In general, the dosage forms of compositions of this disclosure can be prepared by techniques described in Remington's Pharmaceutical Sciences, a reference in this field [Gennaro A R, Ed. Remington: The Science and Practice of Pharmacy. 20th Edition. Baltimore: Lippincott, Williams & Williams, 2000]. For therapeutic purposes, the active components of this combination therapy application can be combined with one or more adjuvants appropriate to the indicated route of administration. The components may be admixed with lactose, sucrose, starch powder, cellulose esters of alkanoic acids, cellulose alkyl esters, talc, stearic acid, magnesium stearate, gelatin, acacia gum, sodium alginate, polyvinylpyrrolidone, and/or polyvinyl alcohol, and then tableted or encapsulated for convenient administration, the amounts of which are ascertainable by the skilled artisan. Such capsules or tablets may contain a controlled-release formulation as may be provided in a dispersion of active compound in hydroxypropyl methylcellulose. Solid dosage forms can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract. Both the solid and liquid oral dosage forms can contain coloring and flavoring to increase patient acceptance. Other adjuvants and modes of administration can be utilized, and these aspects can also be applied to any of the nutritional or dietary supplements described herein.

In some embodiments, the composition can comprise O-CPs for treating or preventing bacterial infections, which can elevate the expression levels of activation markers CD25 and CD69 of the cells such as, but not limited to, NK cells, NKT cells, T lymphocytes, and Non-NK non-T cells in a subject. The activation marker CD69 of monocytes can also have elevated levels during bacterial infections in the subject. The bacterial infection can be a bacterial lipopolysaccharides (LPS) induced infection. In other embodiments, the composition can comprise O-CPs for treating or preventing viral infections which can elevate the expression levels of activation markers CD25 and CD69 of the cells such as, but not limited to, NK cells, NKT cells, and Non-NK non-T cells in the subject. The activation marker CD69 of T lymphocytes and monocytes can also have elevated levels during viral infections in the subject. The viral infection can be an infection induced by viral-mimic Polyinosinic:polycytidylic acid (Poly I:C). In other embodiments, the composition can comprise O-CPs which can elevate the expression levels of activation markers CD25 and CD69 of the cells such as, but not limited to, NK cells, NKT cells, T lymphocytes, and Non-NK non-T cells in the subject, in absence of an immune challenge. The subjects disclosed herein can be specific cell types or model cell type such as, but not limited to, human red blood cells (RBC). In other embodiments, the composition can comprise O-CPs in combination with zinc gluconate and/or zinc picolinate for treating or preventing bacterial or viral infections in a subject.

As disclosed herein, CD69 refers to the earliest inducible cell surface glycoprotein during lymphoid activation resulting in lymphocyte proliferation and cellular signaling. As disclosed herein, CD25 refers to the receptor for the cytokine interleukin-2 (IL-2) which is present on activated T cells and B cells. CD25 can also be expressed on NK and NKT cells, and in some cases shows an inverse correlation with CD69 expression.

The O-CPs in the compositions disclosed herein can stimulate an increase in CD69+CD25+ double-positive cells during the LPS induced infection, during the infection induced by viral-mimic Poly I:C, or in absence of any immune challenge.

As disclosed herein, Poly I:C is referred as a synthetic double-stranded RNA as a model for viral activation through toll-Like-Receptor 3 (TLR3). As disclosed herein, LPS is referred as the highly inflammatory bacterial lipopolysaccharide LPS (bacterial toxin) from E. coli which is used to induce inflammation after treating the immune cells with a natural product.

In some embodiments, the composition can comprise O-CPs for treating or preventing the bacterial or viral infections by determining the expression levels of immune-activating cytokines, anti-inflammatory cytokines, and a growth factor. The immune-activating cytokines disclosed herein can be, such as, interleukin 1 beta (IL-1β), interleukin 6 (IL-6), interleukin 8 (IL-8), macrophage inflammatory protein 1 alpha (CCL3) (MIP-1α), macrophage inflammatory protein 1 beta (CCL4) (MIP-1(3), interferon gamma (IFN-γ), or tumor necrosis factor alpha (TNF-α). The anti-inflammatory cytokines disclosed herein can be, such as, interleukin-1 receptor antagonist (IL-1ra), or interleukin 10 (IL-10). The growth factor disclosed herein can be, such as, granulocyte colony-stimulating factor (G-CSF). In other embodiments, the composition can comprise O-CPs which can elevate the expression levels of IL-1β, IL-6, MIP-1β, TNF-α, IL-1ra, IL-10, or G-CSF during the bacterial infection. In other embodiments, the composition can comprise O-CPs, which can elevate the expression levels of IL-1β, IL-6, IL-8, MIP-1α, MIP-1β, TNF-α, IFN-γ, IL-1ra, IL-10, or G-CSF during the viral infection.

Some embodiments provide methods of predicting or monitoring whether a subject suffering from the bacterial or viral infections responds to a treatment with the composition comprising O-CPs by determining the expression levels of activation markers CD25 and CD69 of the cells such as, but not limited to, NK cells, NKT cells, T lymphocytes and Non-NK non-T cells, and by determining the expression levels of immune-activating cytokines, anti-inflammatory cytokines, and growth factor. The immune-activating cytokines disclosed herein can be, by way of non-limiting examples, IL-1β, IL-6, IL-8, MIP-1α, MP-1β, IFN-γ, or TNF-α. The anti-inflammatory cytokines disclosed herein can be, by way of non-limiting examples, IL-1ra, or IL-10. The growth factor disclosed herein can be, by way of non-limiting example, G-CSF.

As disclosed herein, IL-10 refers to an important mediator of inflammation which is produced by activated macrophages as a proprotein which is cleaved by caspase 1. IL-6, as disclosed herein, refers to mostly a pro-inflammatory cytokine. Inhibitor to IL-6 can be developed as drug for rheumatoid arthritis. IL-8, as disclosed herein, refers to a neutrophil chemotactic factor which can be often associated with inflammation and plays complex role in stem cell biology. MIP-1α, as disclosed herein, refers to factors which are produced by macrophages following stimulation with bacterial endotoxins. These are crucial for immune responses to infection and inflammation. Also, they activate neutrophils and induce the release of pro-inflammatory cytokines. MIP-1β, as disclosed herein, also refers to factors which are produced by macrophages following stimulation with bacterial endotoxins. These are also crucial for immune responses to infection and inflammation. Also, they activate neutrophils and induce the release of pro-inflammatory cytokines. IFN-γ, as disclosed herein, refers to macrophage-activating factor which are associated with a number of autoinflammatory and autoimmune diseases. TNF-α, as disclosed herein, refers to adipokine involved in systemic inflammation which are produced mainly by activated macrophages. They refer to member of a group of cytokines that stimulates acute phase reaction.

As disclosed herein, IL-1ra refers to a natural inhibitor of the pro-inflammatory effects of IL-1β. IL-10, as disclosed herein, refers to an anti-inflammatory cytokine but which requires activation of cells to induce.

As disclosed herein, G-CSF refers to a glycoprotein which promotes proliferation of neutrophils. G-CSF can be used pharmaceutically to mobilize stem cells and accelerate tissue repair, for example after stroke, heart attack, and in many other clinical situations or conditions.

Some embodiments provide methods of treating, preventing or ameliorating age-related somatic diseases in a subject which includes identification of the subject having or at a risk of developing an age-related somatic disease, and administration of a therapeutically effective amount of a pharmaceutical composition comprising O-CPs, or combination of O-CPs with NR, to the subject. The age-related somatic diseases include coronary heart disease, dementia, hypertension, and neurodegenerative diseases. Other embodiments provide methods of treating, preventing, or ameliorating bacterial or viral infections in a subject which includes identification of a subject having bacterial or viral infections, and administration of a therapeutically effective amount of a composition comprising O-CPs, or combination of O-CPs with zinc picolinate and/or selenomethionine, to the subject. The bacterial infection can be a lipopolysaccharides (LPS) induced infection.

Provided herein are methods of treatment of the conditions or infections enumerated above by providing to a subject of a therapeutically effective amount of the O-CPs, or O-CPs in combination with NR, or O-CPs in combination with zinc picolinate and/or selenomethionine, as disclosed herein. In the various embodiments disclosed herein, the subject can be a mammal such as an animal, including, but not limited to, mouse, rat, rabbit, guinea pig, dogs, cats, cows, pigs, sheep, goats, chickens, horses, llamas, alpacas, ducks, etc., and a human.

As used herein, the term “treating” or “treatment” means remediation or amelioration of a medical condition, or alleviation of any undesired signs or symptoms of a disease or illness to an extent, or inhibiting the progression of the disease or illness, or even prevention of the disease or illness, or to improve the condition due to the disease or illness can be considered as the treatment. The term “treatment” does not necessarily mean complete cure of the disease or illness. In addition to treatment or prevention, the compositions disclosed herein can be used to ameliorate or reduce the effects of an ailment, such as for example, when used as a dietary supplement or nutraceutical agent or composition. In addition, compositions disclosed herein can be used to supplement the diet to aid in the reduction of the likelihood of developing an ailment as described herein, such as when used as a dietary supplement to aid in the reduction of a bacterial or viral infection.

The compositions disclosed herein can be assayed in vitro and in vivo for the desired therapeutic or prophylactic activity prior to use in human subjects. For example, in vivo assays can be used to determine whether administration of a specific compound described herein or a combination of compositions disclosed herein is preferred for treating, preventing, or ameliorating age-related somatic disease, bacterial and/or viral infections/diseases, and diseases associated with oxidative stress, or symptoms associated with any of the foregoing. The compositions disclosed herein can also be shown to be effective and safe in animal models or animal subjects. The compositions disclosed herein are shown to be effective and safe in the in-vitro model cells such as, but not limited to, human red blood cells (RBC).

In addition, in vitro assays can be employed to help in identifying optimal dosage ranges. The precise dose to be employed in the compositions can be dependent upon the route of administration, and the seriousness of the disease or illness, and can be decided as per the decision of the practitioner. Suitable dosage ranges of O-CPs to be administered in PBMC are generally about 0.039 g/L-2.5 g/L. In preferred embodiments, the dosage range is about 0.25 g/L-2.0 g/L. Techniques for administration of the compositions described herein are known to person skilled in the art.

### EXAMPLES

The following examples are intended as illustrative and non-limiting and represent specific embodiments of the present disclosure.

### Example 1

### Characterization of PC Solution

Photometric Characterization

The ratio of absorbance at 620 nm and 280 nm is commonly used as an indicator of PC purity. Food-grade C-PC exhibits an A₆₂₀/A₂₈₀ of about 0.7, while analytical (“pure”) C-PC has an A₆₂₀/A₂₈₀ of above 4. The stated specification for the crude PC solution was an A₆₂₀/A₂₈₀ of not less than 1. The extinction coefficient at 615 nm was used for photometric quantification of PC (5.92 mL mg⁻¹ cm⁻¹). For all photometric measurements the PC solutions (phosphate buffer pH 7.0) were diluted to give absorbances of no more than 1 in a 1^{-cm} cuvette. In particular, a solution of 0.5 g of crude PC was prepared in 100 mL of 10 mM sodium phosphate buffer (pH 7) at room temperature. Optical absorbances at 280, 615, and 620 nm was measured in a 1-cm light path quartz cuvette against a blank of 10 mM sodium phosphate buffer (pH 7). The ratio of the absorbances at 620 and 280 nm (A₆₂₀/A₂₈₀) was not less than 1. The concentration of PC in the sample solution was calculated from the absorbance at 615 nm using an extinction coefficient of 5.92 mL mg⁻¹ cm⁻¹. The per-mass percentage of C-PC was not below 30%.

Content of Low Molecular Weight Compounds

10.0 g of crude PC was dissolved in 100 mL of demineralized water. The resulting solution was transferred to 10-kDa molecular weight cut-off (MWCO) dialysis tubing and dialyzed for 24 hours against five changes of 5 liters of demineralized water at room temperature. The contents of the dialysis tubing were then quantitatively transferred to a polyethylene dish, frozen at −70° C., freeze-dried, and weighed. The difference in weight between the original amount of PC (10.0 g) and the amount remaining after dialysis was interpreted as material smaller than 10 kDa. The low molecular weight content was not more than 45% mass of the PC.

The PC solution was used for processing when the results of the photometric characterization conformed to the expected specifications and the PC solution was discarded when the results were not conformed to the expected specifications.

### Example 2

### Preparation of Protein Fraction

Preparation of Crude PC Solution:

After passing quality control or photometric characterization of the crude PC solution, the crude PC solution was dissolved in demineralized water to a concentration of 50 g/L. In particular, 1 kg of PC is dissolved in 20 L of demineralized water at room temperature. Moderate agitation is used for 15 minutes (50-100 rpm, single 60-mm diameter six-blade Rushton stirrer). Introduction of air into the solution was avoided to prevent foaming. The solution was made homogeneous and free of clumps.

Tangential Flow Filtration:

Compounds less than 10 kDa in size were removed from the crude PC solution by filtration, such as tangential flow ultrafiltration, and discarded. Tangential flow ultrafiltration was carried out at room temperature using a 0.6 m² (6.5 ft²) 10-kDa molecular weight cut-off (MWCO) Hydrosart cassette at 0.5 bar (7.25 psi) pressure and a tangential flow rate of about 5-10 L/min (1.3-2.6 gal/min), yielding a filtration rate of about 0.5-1 L/min (0.13-0.26 gal/min). The filtration was continued until the volume of the retentate, or protein fraction was reduced to one quarter of the starting volume, that is, 5 L. The retentate or the protein fraction was washed five times by adding 5 L of demineralized water and the retentate volume or the protein fraction was reduced to 5 L again. This reduced the amount of low molecular weight compounds to below 1% of the starting value by repeated washing as calculated by:

$${\%}_{remaining} = 100 \% \times \frac{{V}_{protein fraction}}{{V}_{initial}} \times {( \frac{{V}_{protein fraction}}{{V}_{protein fraction} + {V}_{wash}} )}^{number of washes}$$

The filtrate thus obtained was clear, and colorless or yellowish to slight green in color. The protein fraction thus obtained was used immediately or was optionally stored frozen at −30° C. (−22° F.), or freeze dried. The filtrate was discarded as non-hazardous aqueous waste.

### Example 3

### Hydrolysis

Preparation of Solution:

The concentration of the retentate or protein fraction obtained in the previous step was adjusted to 50 g/L dissolved >10 kDa solids using demineralized water. The volume required for this can be calculated from the values obtained from the characterization of the PC solution. The pH of the solution was then adjusted to a value of 1.2 using 32% food-grade hydrochloric acid. This corresponded to a final concentration of about 80 mM HCl. The acid was added in small batches with good agitation to prevent clumping. The resistance of the reaction vessel to low pH and chloride was also confirmed.

Pepsin Hydrolysis:

Porcine pepsin was added to the protein fraction to a final concentration of 1000 FIP units/L (that is, 0.5 g/L at a standard activity of ≥2,000 FIP units per gram). After initial mixing, or agitation further pH adjustment was not required, but the reaction was carried out in a closed vessel to avoid loss of HCl to the atmosphere. The progress of hydrolysis was monitored by High-pressure liquid chromatography (HPLC) or photometrically and was completed after 36 hours at 37° C. (98.6° F.). The crude hydrolysate thus obtained was not stored before progressing to the next stage. No aqueous waste was generated during this step.

### Example 4

### Neutralization of the Crude Hydrolysate

Preparation of Solution:

The acidic crude hydrolysate was adjusted to pH 7 using food-grade sodium hydroxide solution to obtain a neutralized crude hydrolysate. Care had been taken to not overtitrate as alkaline pH values have a negative effect on product stability.

### Example 5

### Downstream Processing to Obtain (O-CPs)

Tangential Flow Filtration:

The neutralized crude hydrolysate was immediately subjected to filtration, such as tangential flow ultrafiltration (10 kDa MWCO) to separate the desired O-CPs of less than 10 kDa in the permeate from the unwanted insoluble contaminants, pepsin, and unhydrolyzed protein in the retentate. This was carried out at room temperature using a 0.6 m² (6.5 ft²) 10-kDa MWCO Hydrosart (Sartorius) cassette at 0.5 bar (7.25 psi) pressure and a tangential flow rate of about 5-10 L/min (1.3-2.6 gal/min), yielding a filtration rate of about 0.5-1 L/min (0.13-0.26 gal/min). The filtration was continued until the volume of the retentate was reduced as much as possible (less than one liter). The retentate was optionally washed to recover any remaining <10 kDa peptide, but as this introduced additional water that was required to be removed by freeze-drying, the additional drying costs was weighed against the value of the recovered product. The waste retentate was discarded as non-hazardous aqueous waste. No quality control release measures were required for this step.

### Example 6

### Freeze Drying

Freezing:

Depending on the concentration, the permeate was prone to foaming if not completely frozen. Therefore, the permeate was filled into stainless steel plates to a fill height of 2.5 cm (1 inch) and frozen at −70° C. (−94° F.) for 12 hours to avoid foaming.

Drying:

The plates were transferred to the freeze drier. Primary drying was done at 0.42 mbar (0.006 psi) and −20° C. (−4° F.), secondary drying was done at the same pressure without heating. The dry powder so obtained was stored in a cool dry place and in the dark. The O-CPs content of the final product or the dried powder was determined photometrically and by HPLC. The final product was released when the results conformed to the expected specifications and the final product was discarded when the results were not conformed to the expected specifications.

### Example 7

### Spray Drying

The permeate obtained after downstream processing can optionally undergo a spray drying method to product a dry powder from the permeate. Spray drying includes, for example, rapidly drying the permeate with a hot gas. The dry powder is then stored in a cool, dry, and dark location. The O-CPs content of the final product or of the dried powder is determined photometrically and/or by HPLC.

### Example 8

PC Product

The basis for establishing the assays was the material (Food grade PC powder) from Delhi Nutraceuticals Pvt Ltd. The C-PC content of this material was previously established to be 32%. All quantifications of pure chromopeptide of PC were based on this determination, that is, 100 g of PC (food grade) corresponds to 32 g of pure C-PC.

### Example 9

PC Precipitation

Many proteins, including PC, can be precipitated by high concentrations of trichloroacetic acid or hydrochloric acid. To determine a suitable HCl concentration, volumes of 0.9 mL of a stock solution of 1 g/L of the crude PC (corresponding to 0.32 g/L PC) were treated by addition of 0.1 mL of various concentrations of HCl to give final concentrations of 0.1 M, 0.25 M, 0.5 M, 1 M, and 2 M HCl. These samples were mixed well and incubated on ice for 10 minutes before centrifugation at 13,000×g for 5 minutes at room temperature. The absorption at 640 nm of the supernatant was recorded as a measure of PC remaining in the solution. A concentration of 1 M HCl was found to give satisfactory precipitation.

### Example 10

Photometric Characterization

The extinction coefficient at 615 nm was used for photometric quantification of PC (5.92 mL mg⁻¹ cm⁻¹). All photometric quantifications of PC were carried out in phosphate buffer at pH 7.0. Spectra were recorded to determine the absorption maximum of O-CPs under acidic conditions. Samples of crude PC were diluted to give absorbances of no more than 1 in a 1-cm cuvette.

### Example 11

Quantitative Digestion of PC to Obtain O-CPs

A known amount of PC was digested to completion with pepsin (24 h) to obtain a known amount of O-CPs. Briefly, 80 μL of protein fraction (5 mg/mL) was added to 760 μL of 84 mM HCl and 35 mM NaCl, pH 1.2, and incubated with 0.4 μg of pepsin (≥2,000 FIP units per gram, CARL ROTH) per μg of protein fraction at 37° C. for 24 hours. After complete digestion, one volume of 10 M HCl was added to nine volumes of digestate or crude hydrolysate, followed by incubation on ice for 10 minutes. Insoluble precipitates were then removed by centrifugation at 13,000×g for 5 minutes at room temperature.

Calculations of O-CPs content were based on the following sequences for the alpha and beta subunits of PC.

Complete digest of the PC was proceeded to the shortest fragments, yielding an average O-CPs molecular weight of 1272 g/mol. In reality, the digest yielded some variation in peptide length for the first fragment of the beta subunit, but the variation in molecular weight was relatively small. An exact quantification of each length variant was not possible at this stage due to the unavailability of the standard material.

Complete digest of one mole of dimeric PC subunit (37,454 g/mol) was taken to yield 3,816 g (3×1272 g/mol) of O-CPs, or 10.2% of the mass of PC. If all of the variable-length peptide were in the longest form instead of the shortest assumed here (AACLRD instead of CL), the 0-CPs yield would be 11.3% instead of 10.2%. Therefore, the error was relatively low, and the current assumption was conservative.

### Example 12

Photometric Quantification of O-CPs

For photometric quantification of O-CPs, 0.1 mL of 10 M HCl was added to 0.9 mL of the digestate or crude hydrolysate, followed by mixing and immediate incubation on ice for 10 minutes. This sample was then centrifuged for 5 minutes at 13,000×g at room temperature. The A₆₄₀ of the supernatant was measured against a water blank in a 1-cm cuvette. The A₆₄₀ should be between 0.05 and 1 for reliable measurements; the sample can optionally be diluted with 1 M HCl if necessary. Mass extinction coefficients of 1.91 mL mg⁻¹ cm⁻¹ and 18.7 mL mg⁻¹ cm⁻¹ should be used to convert the A₆₄₀ value to mg/mL of PC or O-CPs, respectively. This conversion gives the concentration in the sample after addition of HCl for precipitation. Further, to determine the concentration before addition of HCl, the calculated value should be multiplied by 1.11.

### Example 13

Quantification of O-CPs by High-Pressure Liquid Chromatography (HPLC)

HPLC was carried out to achieve good separation of the peaks on the RP-C8 column. The column (Acclaim 120 C8, 5 μm particle size, 120 Å pore size, 2.1 mm×250 mm, THERMO SCIENTIFIC) was equilibrated with 0.1% formic acid in water (buffer A) for 20 minutes at a flow rate of 0.3 mL/min. Samples were prepared by adding 0.1 mL of 10 M HCl to 0.9 mL of digestate or crude hydrolysate, followed by mixing and immediate incubation on ice for 10 minutes. The sample was then centrifuged for 5 minutes at 13,000×g at room temperature. Prepared sample (5 μL) was injected before continuing running with buffer A for 5 minutes. A linear gradient from 0 to 100% of buffer B (0.1% formic acid in acetonitrile) was then applied for the next 15 minutes, followed by 100% B over the next 2 minutes and a return to 100% A over another 3 minutes. Buffer A and buffer B were used as mobile phases. Detection was done at 280 nm and 615 nm. The system was an UltiMate 3000 (DIONEX). Calibration curves in the concentration range 0.3 to 6 mg/mL (PC) or 0.03 to 0.6 mg/mL (O-CPs) were required to be prepared regularly, and at least two standards of known concentration are required to be included in each run sequence for validation. Integration was done over all peaks in the 10-16 minute retention time interval.

### Example 14

HCl-Free Optimization of O-CPs Synthesis and Antioxidant Synthesis

Embodiments of processes omitting HCl were pursued and created as part of the disclosure set forth therein because HCl is incompatible with steel and would require specialized equipment to operate on a large scale. The inventors sought to develop a novel method of making O-CPs in the absence of HCl to yield a method that would allow for improved large-scale manufacture of O-CPs. Therefore, experiments were performed to determine processing conditions for making O-CPs in the absence of HCl, which can more effectively and economically be used on a larger, industrial scale.

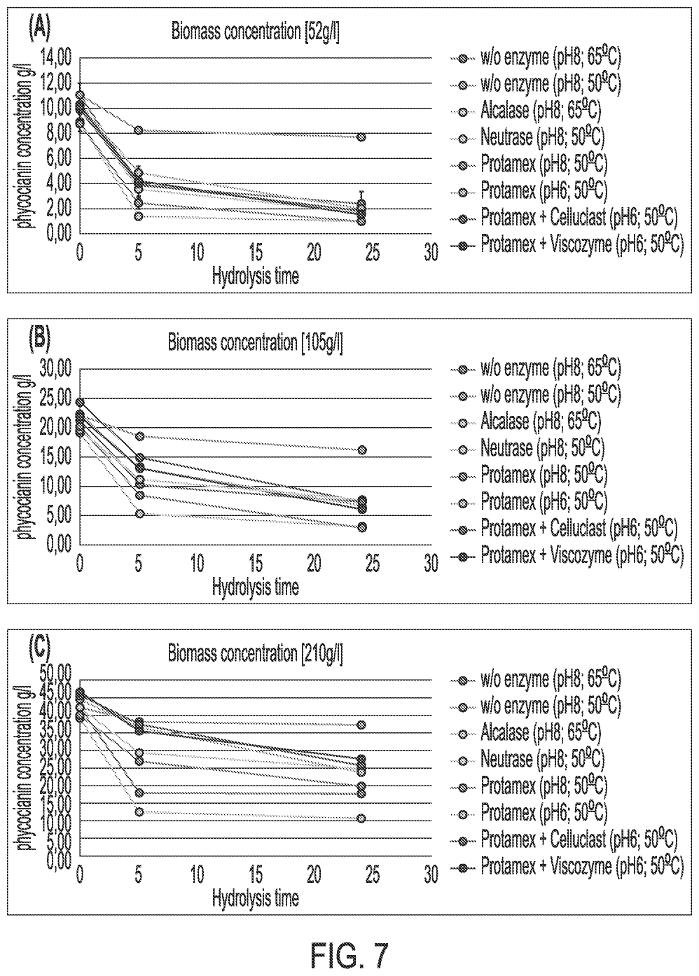

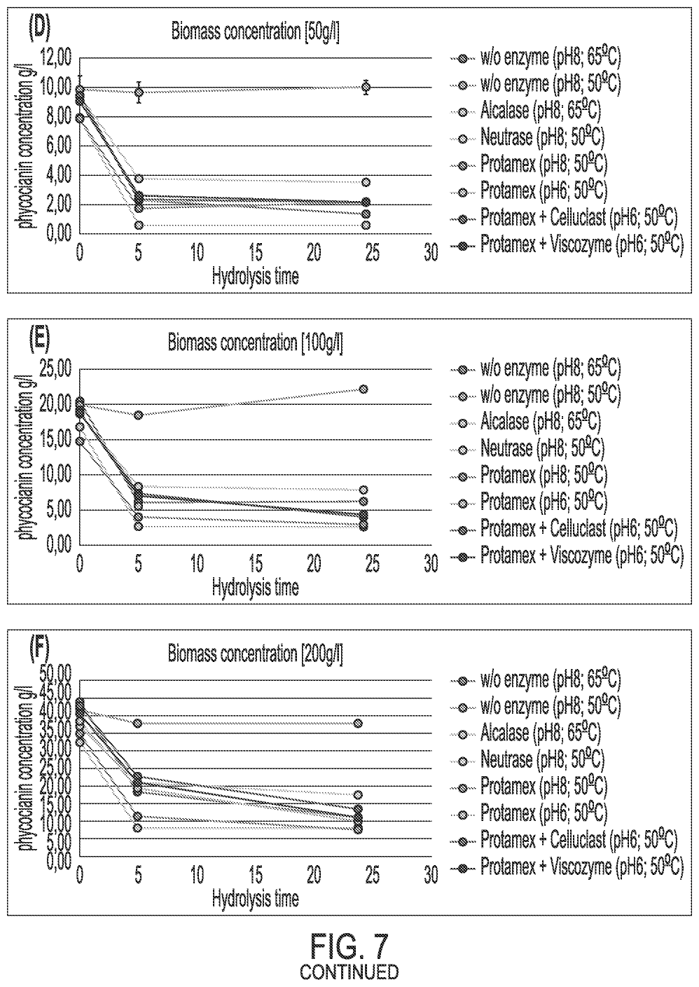

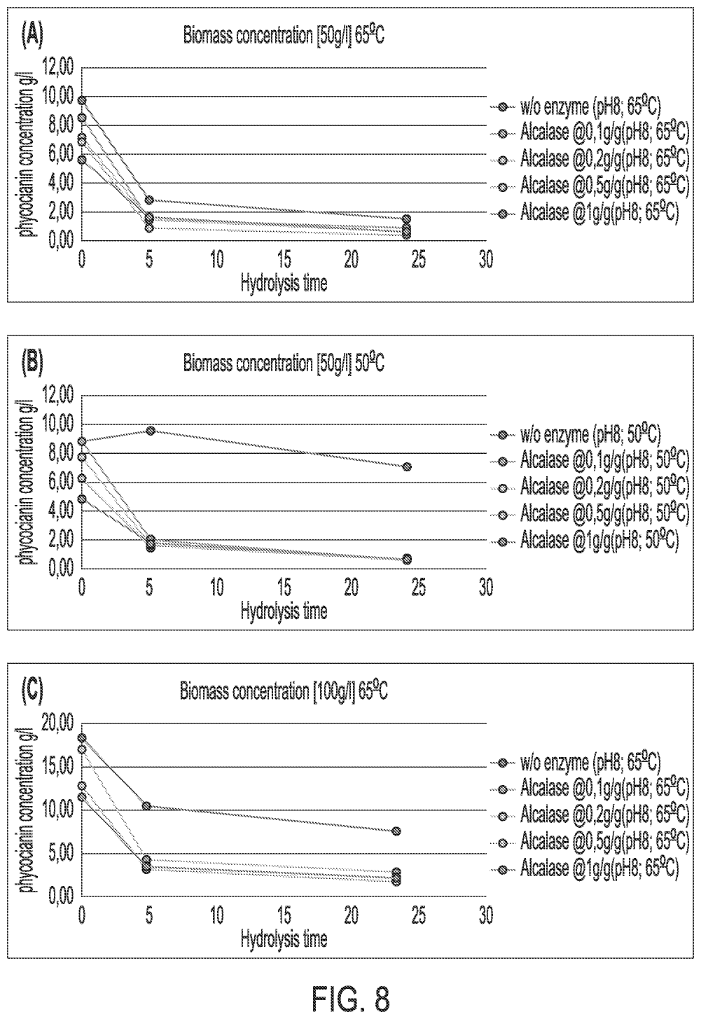

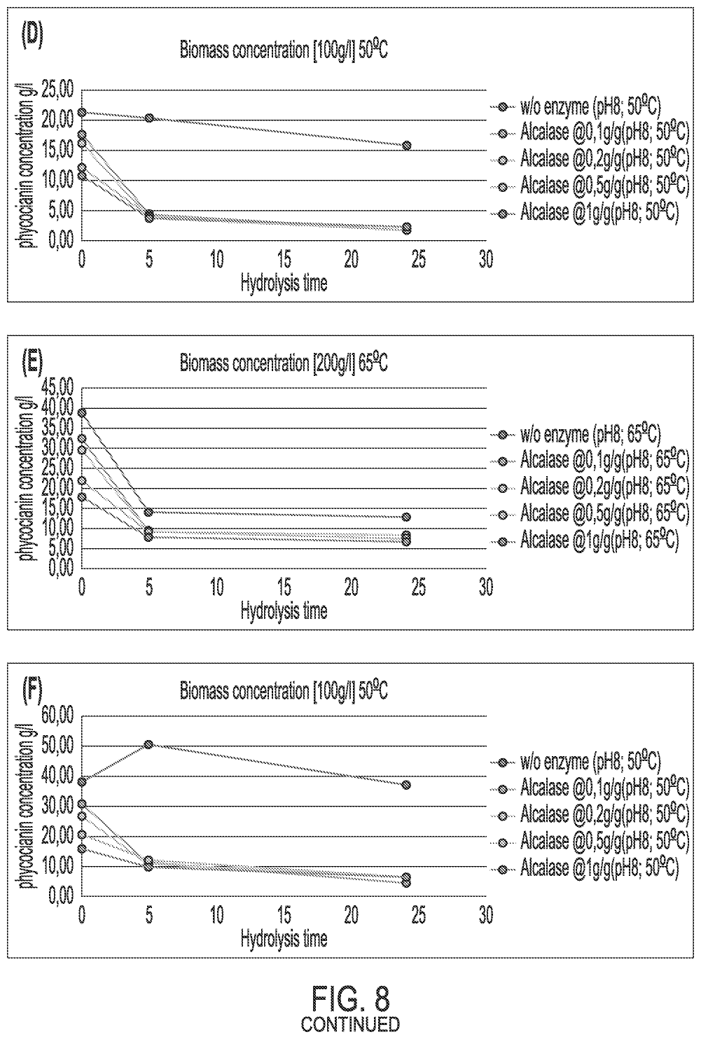

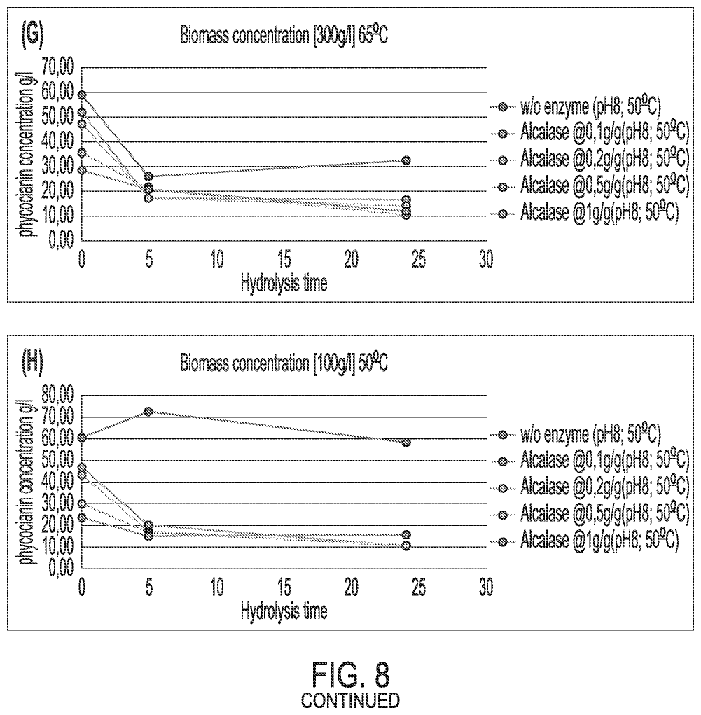

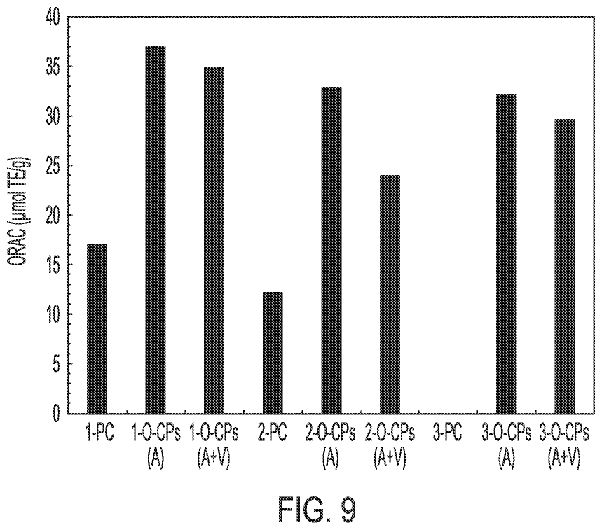

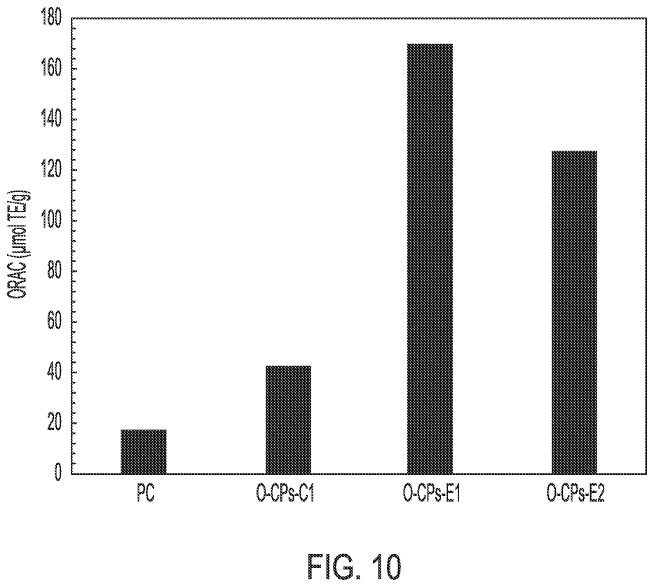

Initial screening was performed to evaluate the importance that crude PC concentration, temperature, pH, reaction time, and enzyme used played on degradation of PC to O-CPs and are shown in FIG. 7. From the initial screening, food-grade alcalase, pH 8, and 65° C. were identified as the optimal conditions, resulting in at least 80% conversion of PC to O-CPs within 5 hr and independent of crude PC composition. A secondary screening was then performed on alcalase to determine an optimal concentration of enzyme, temperature, and pH to use in the hydrolysis reaction to achieve maximal conversion of PC, for which the results are shown in FIG. 8. From the secondary screening, the inventors determined an optimal conditions for the reaction to make O-CPs was a concentration of crude PC in solution of 50 g/L, a pH of 8, 65° C., and 0.2 g alcalase/g of PC. These conditions were utilized to prepare O-CPs from three PC sources. The O-CPs were then evaluated for antioxidant activity (ORAC assay) and are shown in FIG. 9. In FIG. 9, the highest antioxidant activity was found in the O-CPs made by subjecting the PC to alcalase enzyme. A final experiment was performed to further improve on the antioxidant activity of 0-CPs, for which the results are shown in FIG. 10. In FIG. 10, the top best condition was O-CPs made by subjecting PC to alcalase enzyme (0.1 g/g) at 65° C. and pH 8 for 5 hr (O-CPs-E1). This condition yielded a surprising and unexpected result of a 10-fold increase in antioxidant activity over untreated PC, and a 4-fold increase in antioxidant activity over PC subjected to the same conditions absent alcalase (O-CPs-C1).

### Results

A photometric method as well as an HPLC protocol for the quantification of O-CPs had been successfully developed. This study served to identify conditions that allow discrimination between undigested PC and O-CPs and established quantification methods by photometry and HPLC. The O-CPs have been defined as hydrolytic fragments of PC that are acid-soluble and exhibit an absorption maximum between 600 nm and 700 nm.

Discrimination of PC and O-CPs by Acid Precipitation

After addition of HCl to a stock solution of PC and centrifugation, a blue pellet was easily observable at higher concentrations (0.5 M and above), and little to no blue coloration remained in the supernatant. Based on these results, a concentration of 1 M HCl was chosen to precipitate PC while leaving O-CPs in solution. Addition of 1 M HCl to a complete digest of PC did not precipitate any blue material nor lead to a reduction in the A₆₁₅ of the solution, indicating that 0-CPs remain soluble.

Photometric Quantification of O-CPs

A solution containing 0.476 g/L (final concentration) of PC was digested to completion with pepsin for 24 hrs. This corresponded to 0.152 g/L pure PC. A spectrum was recorded after HCl precipitation to determine the absorption maximum under acidic conditions, as shown in FIG. 2. The absorption maximum was at 640 nm, with an extinction coefficient of 1.92 mL mg⁻¹ cm⁻¹ for total PC content. Compared with PC at neutral pH, the maximum was red-shifted, and the extinction coefficient was less than half. As the O-CPs yield from PC was only about 10.2%, the extinction coefficient expressed for O-CPs was 18.8 mL mg⁻¹ cm⁻¹.

A calibration curve was recorded to determine the useful range for using photometry to measure O-CPs concentration. FIG. 3A shows the result for calibration curves for photometric determination of O-CPs expressed in terms of PC concentration. The correlation between A₆₄₀ and concentration was linear up to at least about 0.6 mg/mL PC. FIG. 3B shows the calibration curves for photometric determination of O-CPs expressed in terms of pure O-CPs concentration. The correlation between A₆₄₀ and concentration was linear up to at least about 0.06 mg/mL O-CPs. Quantification was assumed to be reliable for A₆₄₀ values between 0.05 and 1. The slopes of the lines corresponded well with the extinction coefficients determined in the previous section, with 1.92 mL mg⁻¹ cm⁻¹ versus 1.91 mL mg⁻¹ cm⁻¹ for PC and 18.8 mL mg⁻¹ cm⁻¹ versus 18.7 mL mg⁻¹ cm⁻¹ for O-CPs.

Quantification of O-CPs by HPLC

A protocol for the quantification of O-CPs by HPLC was also established. HPLC gives the added advantage of giving an indication of the number of peptides obtained by the complete digest of the protein fraction. Five major peaks were expected to be observed by HPLC. FIGS. 4A and 4B shows chromatograms for a complete PC digest detected at 280 nm and 615 nm. FIG. 4A shows the complete chromatogram, while FIG. 4B only shows the range 10-16 minutes that contains all O-CPs peaks. The PC concentration was 2.89 mg/ml in this sample, corresponding to 0.294 mg/ml O-CPs. Detection at 280 nm was suitable for all peptides containing aromatic residues, particularly tryptophan, while detection at 615 nm was specific for the O-CPs. No peaks at 615 nm were detected outside of the range between 10 and 16 minutes. In FIG. 5 this range of 10-16 minutes was shown for a number of different O-CPs concentrations.

In previous studies, five major and two minor peaks were detected. As it was not possible to assign a specific peptide to each peak, the total area of all peaks was used for quantification. Clear peak detection without manual adjustment was only possible at concentrations of 0.36 mg/mL pure PC (0.037 mg/mL O-CPs) or above up to 5.77 mg/mL PC. FIG. 5 shows the HPLC chromatographs for complete digests of PC based on absorption at 615 nm. The highest peaks corresponded to a digest of 5.77 mg/ml PC, and each lower concentration is half of the one above. The correlation between total area and concentration was highly linear within this range and was used for quantification. The corresponding calibrations are shown in FIGS. 6A and 6B. In particular, FIG. 6A shows the calibration curve for the quantification of 0-CPs by HPLC expressed in terms of PC. FIG. 6B shows the calibration curve for the quantification of O-CPs by HPLC expressed in terms of pure O-CPs content.

Photometric quantification based on different solubilities of PC and O-CPs in HCl was established and found to be easy, reliable, and reproducible. The separation of O-CPs by HPLC was successfully transferred to HPLC setup and gave comparable results in terms of the number of detectable peaks. Quantification of O-CPs by HPLC was also found to be reliable. Both techniques showed very satisfactory performances and can be used for the quantification of PC hydrolysis and O-CPs concentration measurements.

One caveat that should be taken into consideration is that all standardizations of crude PC concentrations are based on the photometric determination of PC in the crude PC as provided. As the crude PC was not pure and did not have a statement of measured purity in absolute terms, a direct gravimetric standardization was not possible. However, the determined PC content (32%) corresponds well with the not-less-than value of 30% given in the product specifications (characterization of crude PC solution). The calibrations are therefore considered adequate.

### Example 15

### Materials and Methods Used for Determining Anti-Aging Effects of the Compositions

Animals

Wistar rats (n=7 in each group; 8 weeks old) were used and animals were reared at the temperature of (22±2° C.), humidity (55±5%) and a 12/12 hour light/dark cycle. Pellet food and water were provided ad libitum. The experiment was conducted under the protocol approved by Firat University.

Study Design Groups

Two control groups were taken for studies. The normal control group was given phosphate-buffered saline (PBS) and the CORT control was given corticosterone (CORT). Doses of O-CPs and NR were determined based on the Human Equivalent Dose (HED) for Drug Development according to Shin et al. (2010). Conversion of human doses to animal doses was based on body surface area. A conversion factor of 6.17 was used to convert human doses to rat doses. The human equivalent dose was 30 mg and 300 mg for O-CPs and 300 mg for nicotinamide riboside.

O-CPs Doses:

Low: 30 mg/70 kg=0.428 mg/kg/day human*6.17=2.64 mg/kg/day rat

High: 300 mg/70 kg=4.286 mg/kg/day human*6.17=26.44 mg/kg/day rat

Nicotinamide riboside (NR) dose:

300 mg/70 kg=4.286 mg/kg/day human*6.17=26.44 mg/kg/day rat

There were 7 study design groups:

1. Normal Control

2. CORT Control

3. CORT+NR=300 mg (HED)

4. CORT+O-CPs (low)=30 mg (HED)

5. CORT+O-CPs (high)=300 mg (HED)

6. CORT+O-CPs (low)+NR=30 mg+300 mg (HED)

7. CORT+O-CPs (high)+NR=300 mg+300 mg (HED)

CORT (Sigma-Aldrich Co., St. Louis, Mo., USA) was suspended in saline with 0.1% Tween 80% and 0.2% DMSO. Rats in groups 2-7 were injected daily with CORT (40 mg/kg) subcutaneously for 21 consecutive days. All injections were performed in a volume of 2 ml/kg body weight, between 7:00 and 7:10 p.m. The rats were dosed with study product involving various dosages of O-CPs, NR and their combination by oral gavage, which was administered daily for 21 days.

### Example 16

### Biochemical Analysis

Serum levels of glucose, triglycerides, cholesterol, aspartate aminotransferase (AST), alanine aminotransferase (ALT), urea, and creatinine were analyzed with a portable automated chemistry analyzer (Samsung LABGEO PT10, Samsung Electronics Co., Suwon, Korea). Total serum CORT levels, and levels of nicotinamide adenine dinucleotide (NAD+) and NAD+ precursors such as nicotinamide (Nam), tryptophan (Trp), nicotinic acid (Na) and nicotinamide mononucleotide (NMN) and NADPH were measured through the telomerase repeat amplification protocol using a commercial kit following the manufacturer's instructions by ELISA (Elx-800, Bio-Tek Instruments Inc, Vermont, USA). Telomere length in the liver was determined by qPCR. Telomerase activity was measured according to the telomerase repeat amplification protocol using a Trapeze kit (Chemicon/Millipore, Billerica, Mass.). The levels of glutathione (GSH) and reactive oxygen species (ROS) in the rat liver were determined using the corresponding commercial ELISA kits (Wako Pure Chemical Industries) according to the manufacturer's instructions. Serum levels of malondialdehyde (MDA) were analyzed by High Performance Liquid Chromatography (HPLC). Antioxidant enzymes [superoxide dismutase (SOD), catalase (CAT), glutathione peroxidase (GSHPx)] were measured using the relevant commercial kits according to the enzyme-linked immunosorbent assay (ELISA, Elx-800, Bio-Tek Instruments Inc, Vermont, USA) method.

### Example 17

### Western Blot Analyses

The activities of telomere capping genes, including telomeric repeat-binding factor 1 (TRF1), telomeric repeat-binding factor 2 (TRF2), protection of telomeres protein 1a (POT-1a), protection of telomeres protein 1b (Pot-1b), and TRF1-interacting protein 2 (Tin2), liver sirtuin 3 (SIRT3) and sirtuin 1 (SIRT1), nicotinamide phosphoribosyltransferase (NAMPT), and several cytokines (IL-1β, IL-6, IL-8; TNF-α, COX-2) were detected. The expression of brain nuclear factor erythroid 2-related factor 2 (Nrf2), nuclear factor kappa B (NF-κB), presynaptic synapsin I, postsynaptic density protein PSD95, PSD93, brain derived neurotrophic factor (BDNF), and nerve growth factor (NGF) were determined.

### Example 18

### Materials and Methods Used for Determining Anti-Bacterial Effects of the Compositions or Effects on Immune Health

Animals

Six-week-old male BALB/c mice was purchased from the Firat University Experimental Animal Center. All mice were ad libitum access to standard rodent chow and water during the study. Mice were housed in cages throughout the study. The following conditions were maintained: temperature, 22±2° C.; relative humidity, 55±5%; and a 12-hours light: dark cycle. The care and treatment of the animals were in accordance with the guidelines established by the Use of Laboratory Animals and were approved by the Animal Care and Use Committee of Firat University.

Study Design Groups

Two control groups were taken for studies. The normal control group was given PBS and the other control was given lipopolysaccharides (LPS) along with a vehicle, i.e., phosphate buffered saline. Forty-two mice were randomly divided into 6 treatment groups with 7 mice in each group as follows:

1. Normal Control

2. LPS+Vehicle

3. LPS+Selenium=200 μg Selenium (HED) from selenomethionine

4. LPS+Zinc Picolinate=30 mg zinc (HED) from zinc picolinate (20% zinc)

5. LPS+O-CPs=300 mg (HED) O-CPs

6. LPS+O-CPs+Zinc Picolinate+Selenium (using the same doses as groups 3-5).

Selenomethionine, zinc picolinate and O-CPs or vehicle were orally administered 2 weeks before LPS treatment. Control mice received 50 μL of PBS. Subsequently, rats in Groups 2 through 6 received LPS (0.04 mg/kg i.p.) 30 min after active treatment with Se, ZnPic, and O-CPs. Basal temperature readings for all treatments and LPS injections were done between 8.00 and 9.00 GMT to avoid temperature differences due to circadian rhythm. The change in rectal temperature in all rats were recorded 30 min after PBS or LPS injection and at subsequent 30 min intervals for 4 hours. After 6 hours of LPS treatment, the mice were euthanized and their blood, liver and lungs were collected for further analysis.

Doses of selenomethionine, zinc picolinate and O-CPs were determined based on the Human Equivalent Dose (HED) for Drug Development according to Shin et al. (2010). Conversion of human doses to animal doses were based on body surface area. A conversion factor of 12.33 was used to convert human doses to mouse doses. The human equivalent dose was 200 μg of selenium (Se) from selenomethionine, 30 mg of zinc (Zn) from zinc picolinate and 300 mg of O-CPs.

Selenomethionine dose: 200 μg/70 kg=2.86 μg/kg/day human*12.33=35.23 μg/kg/day per mouse;
Zinc picolinate (20%) dose: 30 mg/70 kg=0.428 mg/kg/day human*12.33=5.28 elemental zinc mg/kg/day mouse=5.28*100/20=26.4 mg/kg/day per mouse;
O-CPs dose: 300 mg/70 kg=4.29 mg/kg/day human*12.33=52.84 mg/kg/day per mouse.

### Example 19

### Biochemical Analysis

Serum levels of glucose, triglycerides, cholesterol, aspartate aminotransferase (AST), alanine aminotransferase (ALT), urea, and creatinine were analyzed with a portable automated chemistry analyzer (Samsung LABGEO PT10, Samsung Electronics Co., Suwon, Korea). Serum levels of MDA were analyzed by HPLC. Antioxidant enzymes (SOD, CAT, GSHPx) were measured using the relevant commercial kits according to the enzyme-linked immunosorbent assay (ELISA, Elx-800, Bio-Tek Instruments Inc, Vermont, USA) method. Serum, zinc and selenium levels in liver and lung were detected by atomic absorption spectroscopy (AAS).

### Example 20

### Western Blot Analyses

Pro-inflammatory cytokine, NF-κB, TNFα, IL-1, IL-6, IL-17, COX-2, inducible nitric oxide synthase (iNOS), metallothionein (MT), ZIP1, ZIP4, ZIP14 (zinc importers), ZNT1, ZNT4, and ZNT9 (zinc exporters) in the livers and lungs were detected by Western Blot.

### Example 21

### Histology

The lungs and liver of each mouse were harvested, stored in 10% buffered formalin and embedded in paraffin. For histological investigations, 3 μm sections were cut, deparrafinized, dehydrated and stained with haematoxylin and eosin (H & E). The tissue sections were observed for inflammatory changes under light microscope.

### Example 22

### Tests for Determining Anti-Aging Effects of the Compositions

The samples size of the study was determined to be totally 49 rats (n=7; 7 groups) with the help of G*Power package program (Version 3.1.9.2) with alpha error 0.05 and 90% power with effect size 0.7 calculated from previous studies. The data was analyzed using statistical analysis software (SPSS 17.0). In this study, conformity to the assumption of normality from the prerequisites of the parametric tests was performed using the “Shapiro-Wilk” test and the homogeneity of the variances were checked with the “Levene” test. Analysis of variance (ANOVA) test was performed to determine the differences between the groups and post-hoc Tukey test was used for multiple comparisons of the groups. Statistical significance was accepted as P<0.05.

F tests—ANOVA: Fixed effects, omnibus, one-way

Analysis: A priori: Compute required sample size

Input: Effect size f=0.7
α errprob=0.05
Power (1-β errprob)=0.9
Number of groups=7

Output: Non centrality parameter λ=24.0100000
Critical F=2.3239938
Numerator df=6
Denominator df=42
Total sample size=49
Actual power=0.9483953

### Example 23

### Anti-Aging Effects of PC and NR

Effects of PC and NR on initial and final body weight in corticosterone (CORT) induced aging rats has been shown in below in Table 1. Effects of O-CPs and NR on liver in terms of levels of various antioxidant enzymes and its precursors such as NAD+, Nam, Na and NMN, NADPH and GSH present in corticosterone (CORT) induced aging rats are provided in Table 2. Effects of O-CPs and NR on serum antioxidant enzymes such as MDA, SOD, GSH-Px and CAT in corticosterone (CORT) induced aging rats are provided in Table 3. Effects of O-CPs and NR on serum biochemistry such as glucose (GLU), creatine, blood urea nitrogen (BUN), ALT and AST in corticosterone (CORT) induced aging rats are provided in Table 4.

TABLE 1
Effects of O-CPs (PC30 or PC300) and NR on initial and final body weight in corticosterone (CORT) induced aging rats
Data are presented as mean and standard deviation.

| | Groups | | | | | | |
|---|---|---|---|---|---|---|---|
| Body | | | | CORT + | CORT + | CORT + | CORT + |
| Weight | Control | CORT | CORT + NR | O-CP30 | O-CP300 | NR + O-CP30 | NR + O-CP300 |
| Initial Body | 246.43 ± 8.73 | 249.86 ± 10.09 | 246.86 ± 12.60 | 247.43 ± 18.85 | 247.71 ± 11.31 | 248.43 ± 14.90 | 245.29 ± 16.84 |
| Weight, g | | | | | | | |
| Final Body | 276.57 ± 9.85^{a} | 210.43 ± 8.28^{b} | 222.86 ± 23.11^{b} | 221.29 ± 11.25^{b} | 221.86 ± 9.92^{b} | 225.14 ± 13.23^{b} | 217.86 ± 19.42^{b} |
| Weight, g | | | | | | | |

^{a-b}Means in the same line without a common superscript differ significantly (P < 0.05).

TABLE 2
Effects of O-CPs and NR on liver in corticosterone (CORT) induced aging rats

| | Groups | | | | | | |
|---|---|---|---|---|---|---|---|
| | | | | CORT + | CORT + | CORT + | CORT + |
| Enzymes | Control | CORT | CORT + NR | O-CP30 | O-CP300 | NR + O-CP30 | NR + O-CP300 |
| CORT, | 46.33 ± 5.82^{b} | 110.22 ± 6.15^{a} | 102.16 ± 8.68^{a} | 104.48 ± 3.67^{a} | 105.91 ± 6.39^{a} | 105.46 ± 8.13^{a} | 104.99 ± 5.34^{a} |
| ng/ml | | | | | | | |
| Liver | 0.76 ± 0.05^{a} | 0.33 ± 0.04^{f} | 0.59 ± 0.04^{c} | 0.42 ± 0.05^{e} | 0.49 ± 0.06^{de} | 0.67 ± 0.05^{b} | 0.55 ± 0.06^{cd} |
| Nicotin- | | | | | | | |
| amide | | | | | | | |
| adenine | | | | | | | |
| dinucleotide | | | | | | | |
| NAD+, | | | | | | | |
| μmol/g | | | | | | | |
| Liver | 2.36 ± 0.15^{a} | 1.01 ± 0.08^{e} | 1.60 ± 0.06^{c} | 1.31 ± 0.09^{d} | 1.43 ± 0.13^{d} | 1.78 ± 0.10^{b} | 1.66 ± 0.10^{cb} |
| Nicotin- | | | | | | | |
| amide | | | | | | | |
| (Nam), | | | | | | | |
| μmol/g | | | | | | | |
| Liver | 1.79 ± 0.09^{a} | 1.22 ± 0.12^{d} | 1.63 ± 0.09^{abc} | 1.46 ± 0.10^{c} | 1.57 ± 0.17^{bc} | 1.71 ± 0.13^{ab} | 1.73 ± 0.12^{ab} |
| Nicotinic | | | | | | | |
| acid (Na), | | | | | | | |
| μmol/g | | | | | | | |
| Liver | 0.45 ± 0.05^{a} | 0.15 ± 0.02^{e} | 0.30 ± 0.02^{bc} | 0.20 ± 0.02^{de} | 0.24 ± 0.03^{cd} | 0.36 ± 0.05^{b} | 0.32 ± 0.07^{b} |
| Nicotin- | | | | | | | |
| amide mono | | | | | | | |
| nucleotide | | | | | | | |
| (NMN), | | | | | | | |
| μmol/g | | | | | | | |
| Liver | 62.65 ± 4.13^{a} | 30.84 ± 3.52^{e} | 44.42 ± 2.31^{c} | 39.88 ± 3.04^{cd} | 41.38 ± 3.57^{cd} | 52.52 ± 5.39^{b} | 37.89 ± 2.23^{d} |
| NADPH | | | | | | | |
| nmol/g | | | | | | | |
| Liver GSH | 48.51 ± 3.62^{a} | 16.95 ± 2.81^{e} | 33.25 ± 2.07^{bc} | 24.15 ± 1.70^{d} | 29.74 ± 1.15^{c} | 36.89 ± 2.18^{b} | 32.77 ± 2.66^{c} |
| U/mg protein | | | | | | | |

Data are presented as mean and standard deviation.
^{a-f}Means in the same line without a common superscript differ significantly (P < 0.05)

TABLE 3
Effects of O-CPs and NR on serum antioxidant enzymes in corticosterone (CORT) induced aging rats

| | Groups | | | | | | |
|---|---|---|---|---|---|---|---|
| | | | | CORT + | CORT + | CORT + | CORT + |
| Enzymes | Control | CORT | CORT + NR | O-CP30 | O-CP300 | NR + O-CP30 | NR + O-CP300 |
| MDA nmol/ml | 0.55 ± 0.07^{f} | 1.97 ± 0.13^{a} | 1.74 ± 0.07^{b} | 1.53 ± 0.07^{c} | 1.41 ± 0.07^{cd} | 1.26 ± 0.07^{e} | 1.28 ± 0.09^{de} |
| SOD, U/ml | 128.00 ± 5.48^{a} | 54.54 ± 6.39^{e} | 61.36 ± 4.5^{e} | 72.70 ± 3.05^{d} | 86.41 ± 6.34^{c} | 96.84 ± 6.77^{b} | 98.27 ± 4.65^{b} |
| GSH-Px, U/ml | 64.87 ± 3.08^{a} | 19.62 ± 1.37^{e} | 26.28 ± 1.98^{d} | 31.76 ± 4.96^{cd} | 37.10 ± 2.80^{c} | 44.68 ± 5.58^{b} | 45.98 ± 4.10^{b} |
| CAT, U/ml | 164.98 ± 6.13^{a} | 102.03 ± 4.80^{e} | 114.69 ± 4.82^{d} | 124.55 ± 5.48^{c} | 131.43 ± 3.76^{bc} | 140.55 ± 6.76^{b} | 139.05 ± 7.81^{b} |

Data are presented as mean and standard deviation.
^{a-f}Means in the same line without a common superscript differ significantly (P < 0.05)

TABLE 4
Effects of O-CPs and NR on serum biochemistry in corticosterone (CORT) induced aging rats.

| | Groups | | | | | | |
|---|---|---|---|---|---|---|---|
| | | | | CORT + | CORT + | CORT + | CORT + |
| Enzymes | Control | CORT | CORT + NR | O-CP30 | O-CP300 | NR + O-CP30 | NR + O-CP300 |
| GLU (mg/dl) | 119.14 ± 8.43^{ab} | 127.86 ± 5.05^{a} | 119.86 ± 7.10^{ab} | 118.57 ± 2.64^{abc} | 114.14 ± 5.24^{bc} | 109.29 ± 5.47^{c} | 118.29 ± 4.68^{bc} |
| Creatine | 0.40 ± 0.06 | 0.38 ± 0.07 | 0.37 ± 0.08 | 0.40 ± 0.08 | 0.39 ± 0.06 | 0.36 ± 0.09 | 0.45 ± 0.05 |
| BUN (mg/dl) | 21.46 ± 3.57 | 23.01 ± 2.32 | 22.90 ± 1.87 | 22.13 ± 2.52 | 21.59 ± 3.00 | 22.20 ± 4.11 | 23.17 ± 3.65 |
| ALT (U/L) | 95.57 ± 8.42 | 98.14 ± 12.81 | 98.00 ± 5.55 | 98.71 ± 6.82 | 98.03 ± 7.36 | 96.10 ± 5.78 | 98.72 ± 4.79 |
| AST (U/L) | 110.69 ± 11.79 | 117.46 ± 7.53 | 114.71 ± 10.92 | 112.60 ± 13.13 | 117.43 ± 15.25 | 114.06 ± 12.59 | 116.20 ± 19.06 |

Data are presented as mean and standard deviation.
^{a-c}Means in the same line without a common superscript differ significantly (P < 0.05)

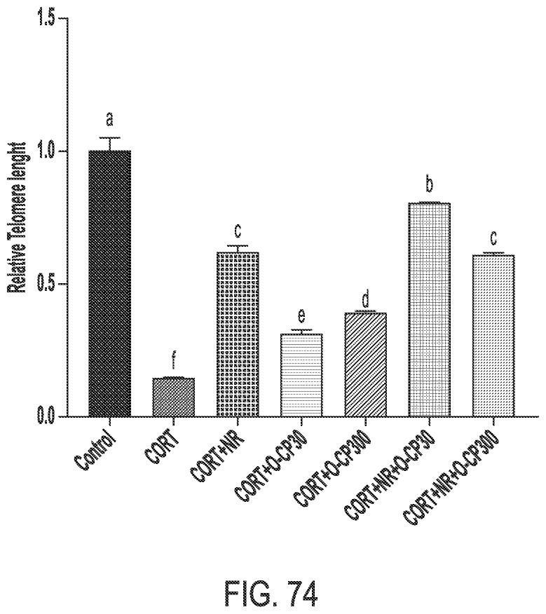

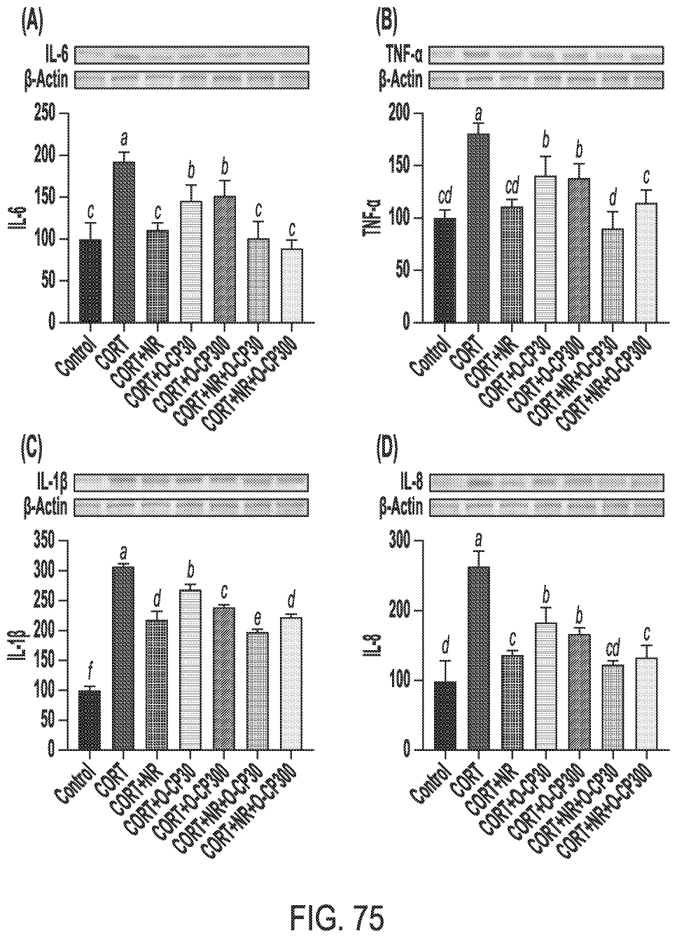

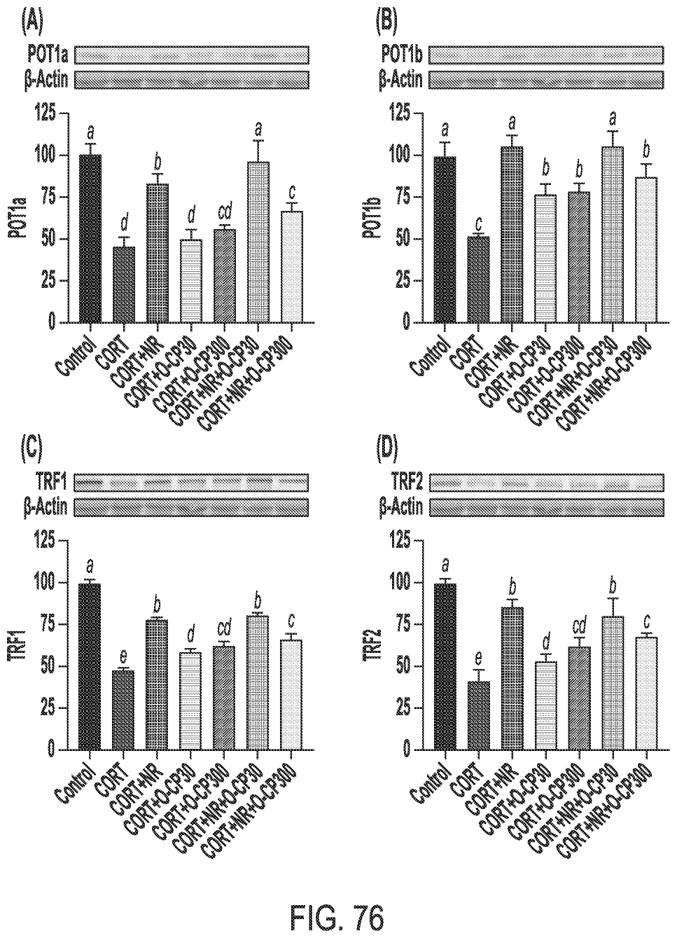

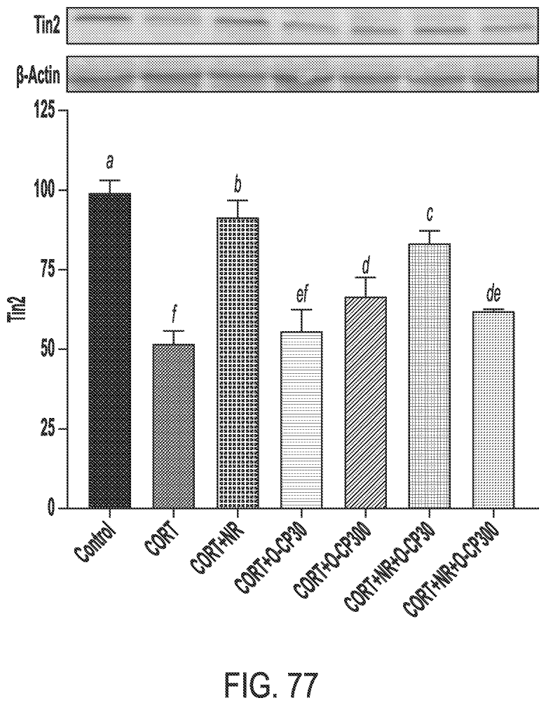

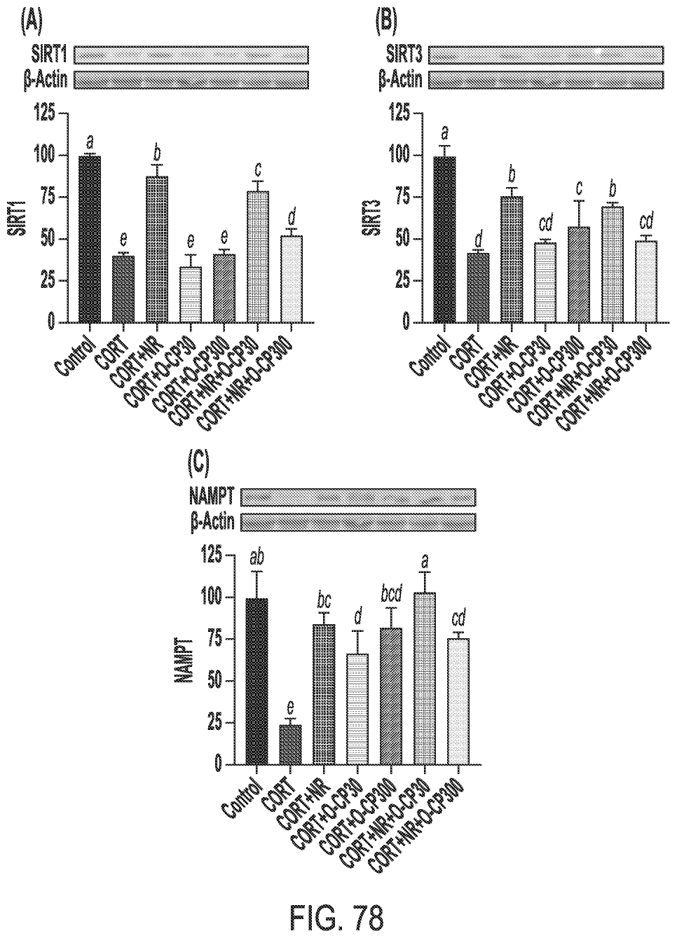

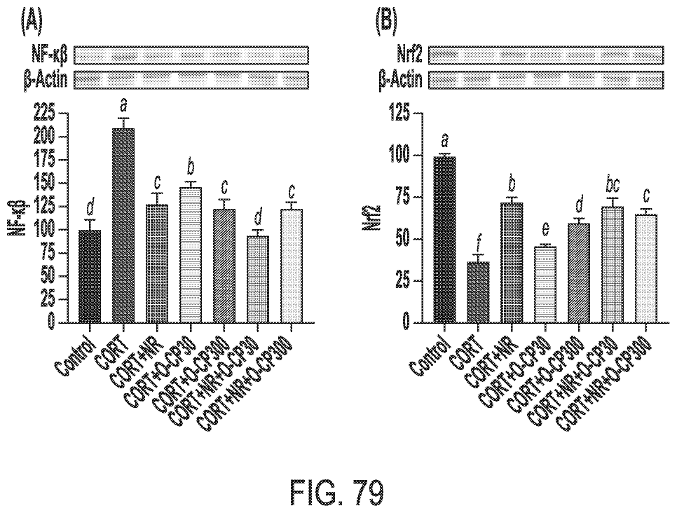

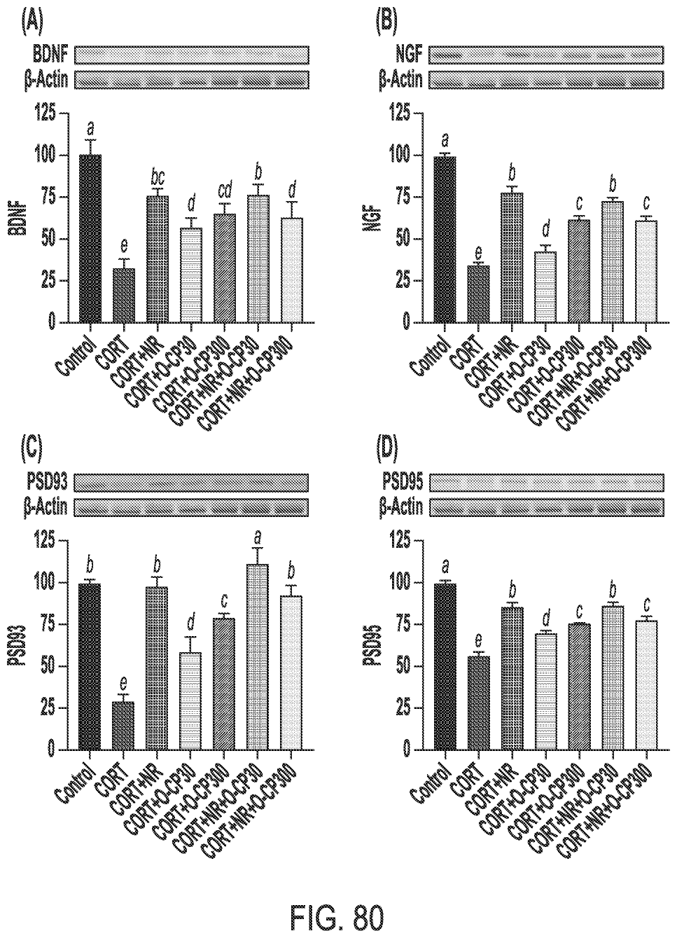

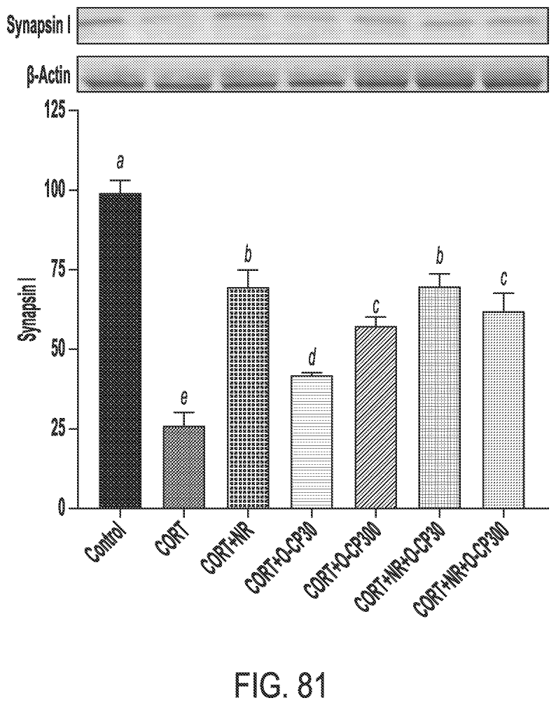

Effects of various dosages of O-CPs, NR and their combination on liver relative telomere length in corticosterone (CORT) induced aging rats are shown in FIG. 74. In FIG. 74, the combination of O-CPs and NR prevents degradation of liver telomere in comparison with CORT control which shows degradation of liver telomere to a greater extent in absence of any treatment. Effects of various dosages of O-CPs, NR and their combination on liver interleukins (IL-6, IL-1β, IL-8) and TNF-α levels in corticosterone (CORT) induced aging rats are shown in FIGS. 75A-75D. Effects of various dosages of O-CPs, NR and their combination on liver POT1a, POT1b, TRF1, TRF2 and Tin2 levels in corticosterone (CORT) induced aging rats are shown in FIGS. 76A-76D and 77. Effects of various dosages of O-CPs, NR and their combination on liver SIRT1, SIRT3 and NAMPT levels in corticosterone (CORT) induced aging rats are shown in FIGS. 78A-78D. Effects of various dosages of O-CPs, NR and their combination on brain NF-κβ, and Nrf2 levels in corticosterone (CORT) induced aging rats are shown in FIGS. 79A-79B. Further, effects of various dosages of O-CPs, NR and their combination on brain BDNF, NGF, PSD93, PSD95 and Synapsin I levels in corticosterone (CORT) induced aging rats are shown in FIGS. 80A-80D and 81.

### Example 24

### Tests for Determining Anti-Bacterial Effects of the Compositions or Effects on Immune Health

The samples size of the study was 42 mice (n=7; 6 groups) and analyzed with the help of G*Power package program (Version 3.1.9.2) with alpha error 0.05 and 85% power with effect size 0.65 calculated from previous studies. The data was analyzed using statistical analysis software (SPSS 17.0). In this study, conformity to the assumption of normality from the prerequisites of the parametric tests was performed using the “Shapiro-Wilk” test and the homogeneity of the variances were checked with the “Levene” test. Analysis of variance (ANOVA) test was performed to determine the differences between the groups and post-hoc Tukey test was used for multiple comparisons of the groups. Statistical significance was accepted as P<0.05.

F tests—ANOVA: Fixed effects, omnibus, one-way

Analysis: A priori: Compute required sample size

Input: Effect size f=0.65
α errprob=0.05
Power (1-β errprob)=0.85
Number of groups=6

Output: Non centrality parameter λ=17.7450000
Critical F=2.4771687
Numerator df=5
Denominator df=36
Total sample size=42
Actual power=0.8726155

### Example 25

### Effects of Se, Zn, and O-CPs on Immune Health

Effects of selenium (Se), zinc (Zn), and O-CPs on serum biochemical parameters such as ALT, AST, ALP, LDH, BUN, Creatine and T Bilirubin, in LPS-induced pyrexia in mice are provided in Table 5. Effects of Se, Zn, and O-CPs on liver and lung levels of antioxidant enzymes such as MDA, SOD, CAT, GSHPx in LPS induced pyrexia in mice, are provided in Table 6.

TABLE 5
Effects of Se, Zn, and O-CPs (PC-O) on serum biochemical parameters in LPS-induced pyrexia in mice

| | Groups | | | | | |
|---|---|---|---|---|---|---|
| | | | | | | LPS + Se + |
| Enzymes | Control | LPS | LPS + Se | LPS + Zn | LPS + PC-O | Zn + PC-O |
| ALT (IU/L) | 41.57 ± 10.061^{e} | 365.29 ± 16.93^{a} | 261.57 ± 16.16^{bc} | 280.43 ± 8.85^{b} | 244.43 ± 16.37^{c} | 220.29 ± 18.74^{d} |
| AST (IU/L) | 52.29 ± 5.56^{e} | 779.29 ± 59.38^{a} | 573.43 ± 59.23^{b} | 580.86 ± 32.61^{b} | 517.57 ± 38.20^{bc} | 458.00 ± 50.36^{c} |
| ALP (IU/L) | 59.86 ± 15.04^{d} | 318.43 ± 31.63^{a} | 285.43 ± 10.33^{b} | 292.71 ± 15.34^{ab} | 266.71 ± 12.65^{bc} | 243.14 ± 9.82^{c} |
| LDH, (IU/L) | 66.09 ± 5.84^{e} | 277.59 ± 16.03^{a} | 192.11 ± 10.72^{c} | 216.13 ± 11.39^{b} | 189.83 ± 5.39^{c} | 170.41 ± 6.71^{d} |
| BUN, (mg/dL) | 19.46 ± 1.50^{c} | 40.87 ± 4.92^{a} | 34.36 ± 3.26^{b} | 35.57 ± 3.20^{ab} | 32.44 ± 2.89^{b} | 32.03 ± 3.64^{b} |
| Creatine, (mg/dL) | 0.42 ± 0.06^{c} | 0.70 ± 0.07^{a} | 0.59 ± 0.08^{ab} | 0.59 ± 0.06^{ab} | 0.55 ± 0.06^{b} | 0.52 ± 0.09^{bc} |
| T Bilirubin, (mg/dL) | 0.29 ± 0.06^{d} | 1.20 ± 0.12^{a} | 0.95 ± 0.08^{b} | 0.97 ± 0.08^{b} | 0.90 ± 0.05^{bc} | 0.82 ± 0.04^{c} |

Data are presented as mean and standard deviation.
^{a-e}Means in the same line without a common superscript differ significantly (ANOVA and Tukey's post-hoc test; P < 0.05).

TABLE 6
Effects of Se, Zn, and O-CPs on liver and lung levels of antioxidant enzymes in LPS induced pyrexia in mice

| | Groups | | | | | |
|---|---|---|---|---|---|---|
| | | | | | | LPS + Se + |
| Enzymes | Control | LPS | LPS + Se | LPS + Zn | LPS + PC-O | Zn + PC-O |
| Liver, MDA, (nmol/mL) | 3.24 ± 0.22^{e} | 7.11 ± 0.46^{a} | 5.86 ± 0.26^{b} | 5.92 ± 0.35^{b} | 5.16 ± 0.34^{c} | 4.34 ± 0.36^{d} |
| Liver, SOD, (U/mg) | 153.7 ± 9.11^{a} | 56.37 ± 5.29^{d} | 95.16 ± 5.06^{b} | 75.94 ± 5.93^{c} | 77.19 ± 7.29^{c} | 103.35 ± 6.74^{b} |
| Liver, CAT, (U/mg) | 86.89 ± 5.45^{a} | 31.49 ± 2.57^{d} | 41.89 ± 4.03^{c} | 43.06 ± 3.24^{c} | 42.03 ± 3.26^{c} | 60.71 ± 3.54^{b} |
| Liver, GSHPx, (U/mg) | 61.32 ± 3.77^{a} | 21.39 ± 1.96^{e} | 44.79 ± 3.86^{c} | 36.86 ± 1.98^{d} | 37.98 ± 2.16^{d} | 49.64 ± 3.56^{b} |
| Lung, MDA (nmol/mL) | 4.18 ± 0.44^{d} | 9.24 ± 0.42^{a} | 6.67 ± 0.49^{b} | 6.83 ± 0.30^{b} | 6.69 ± 0.37^{b} | 5.89 ± 0.37^{c} |
| Lung, SOD, (U/mg) | 105.78 ± 6.72^{a} | 31.89 ± 2.86^{d} | 59.32 ± 4.81^{b} | 50.36 ± 2.93^{c} | 49.28 ± 5.04^{c} | 65.75 ± 3.93^{b} |
| Lung, CAT, (U/mg) | 52.30 ± 3.70^{a} | 18.69 ± 1.43^{d} | 32.29 ± 1.86^{b} | 28.53 ± 1.70^{c} | 27.76 ± 2.76^{c} | 35.12 ± 1.75^{b} |
| Lung, GSHPx, (U/mg) | 46.15 ± 3.97^{a} | 16.86 ± 1.42^{d} | 33.86 ± 3.41^{b} | 28.08 ± 1.98^{c} | 26.63 ± 2.46^{c} | 36.75 ± 2.53^{b} |

Data are presented as mean and standard deviation.
^{a-e}Means in the same line without a common superscript differ significantly (ANOVA and Tukey's post-hoc test; P < 0.05).

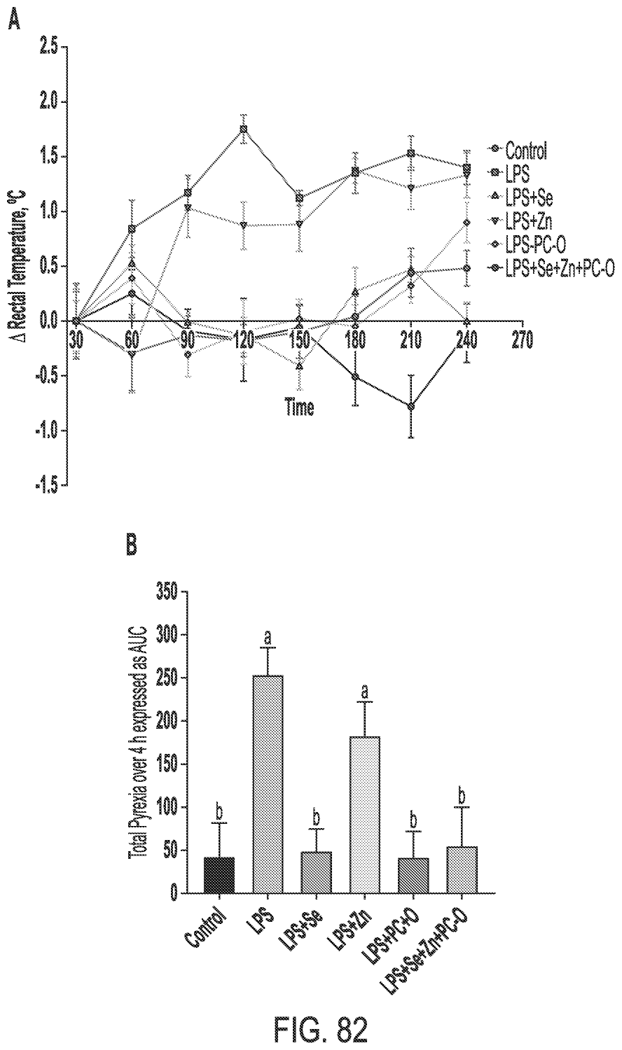

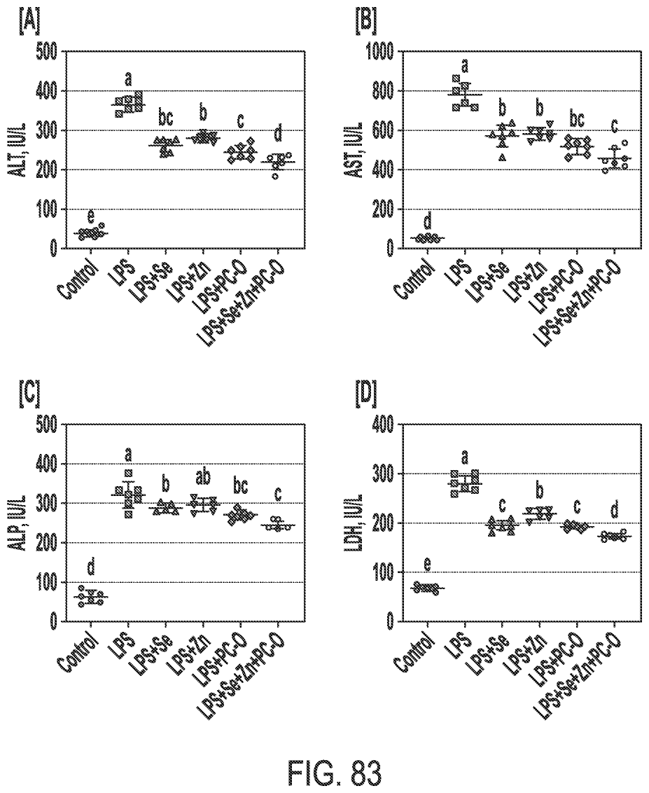

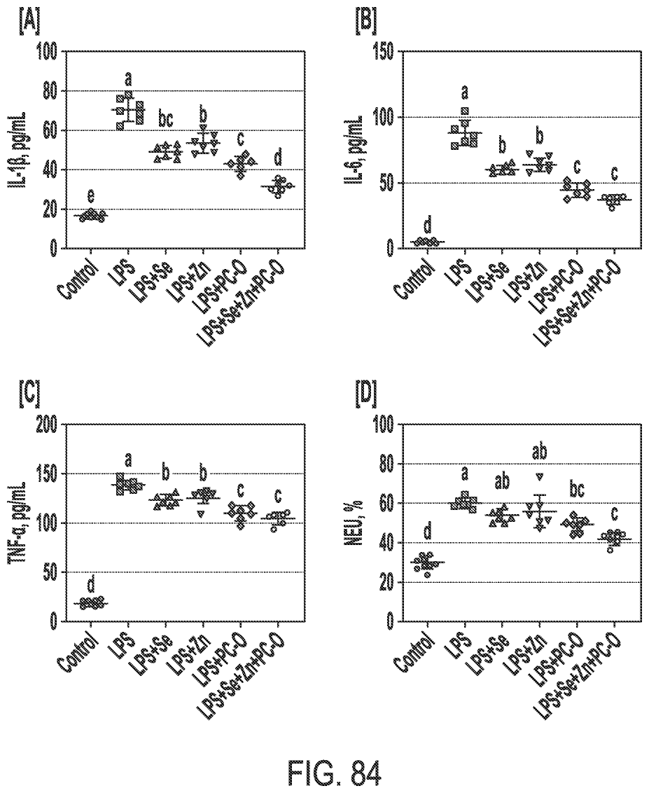

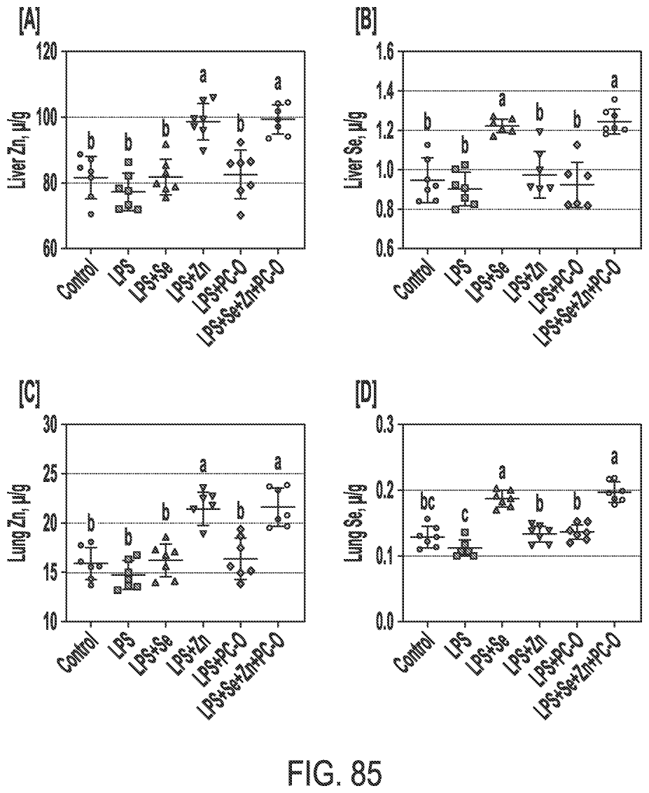

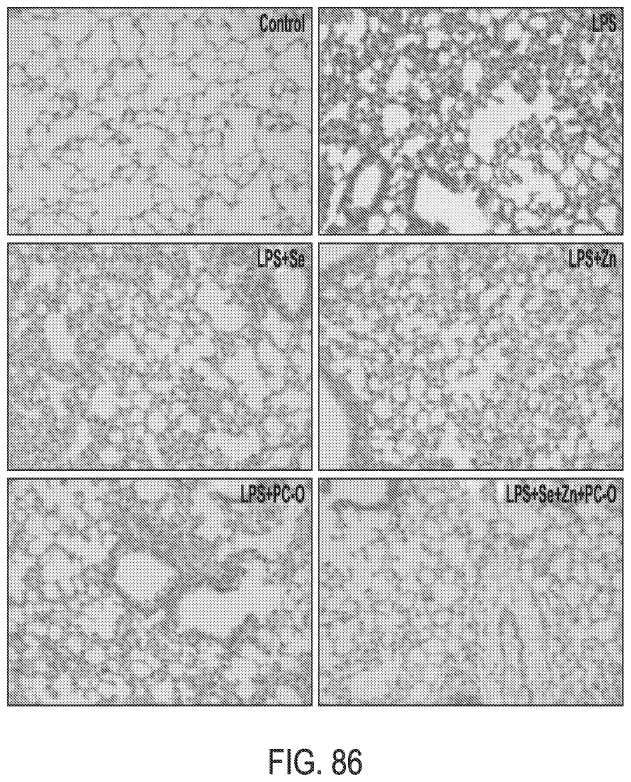

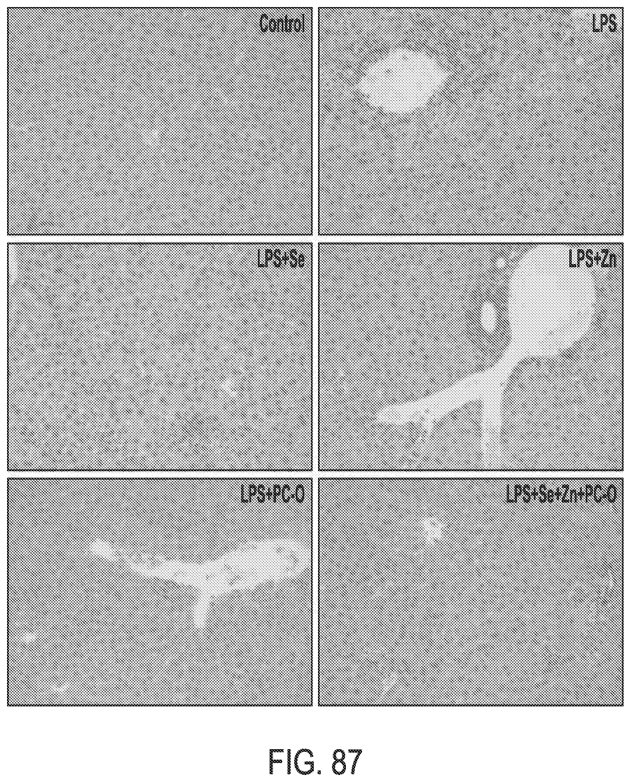

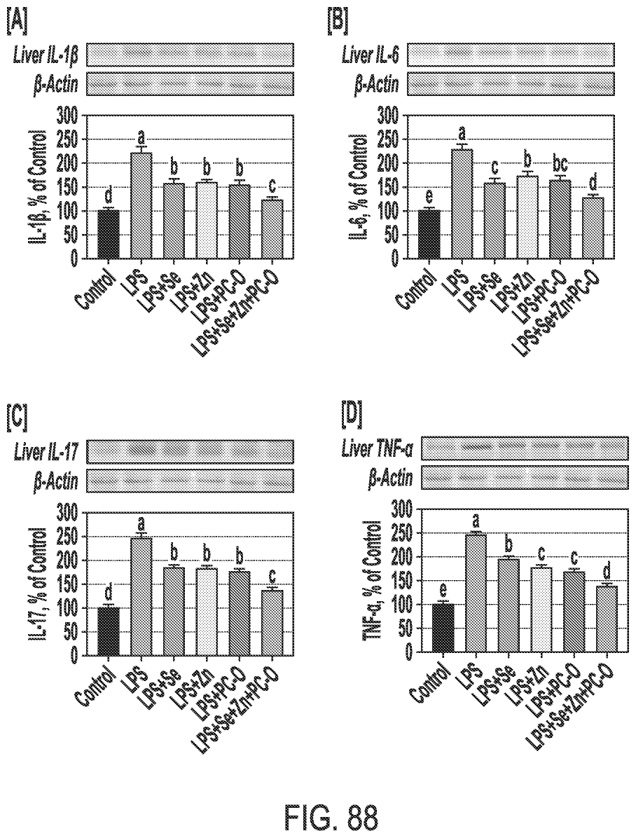

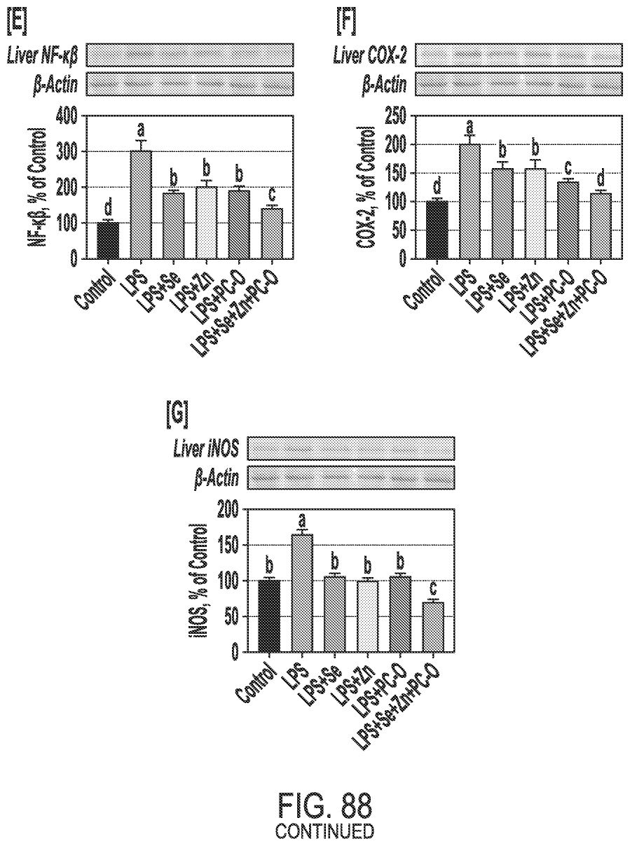

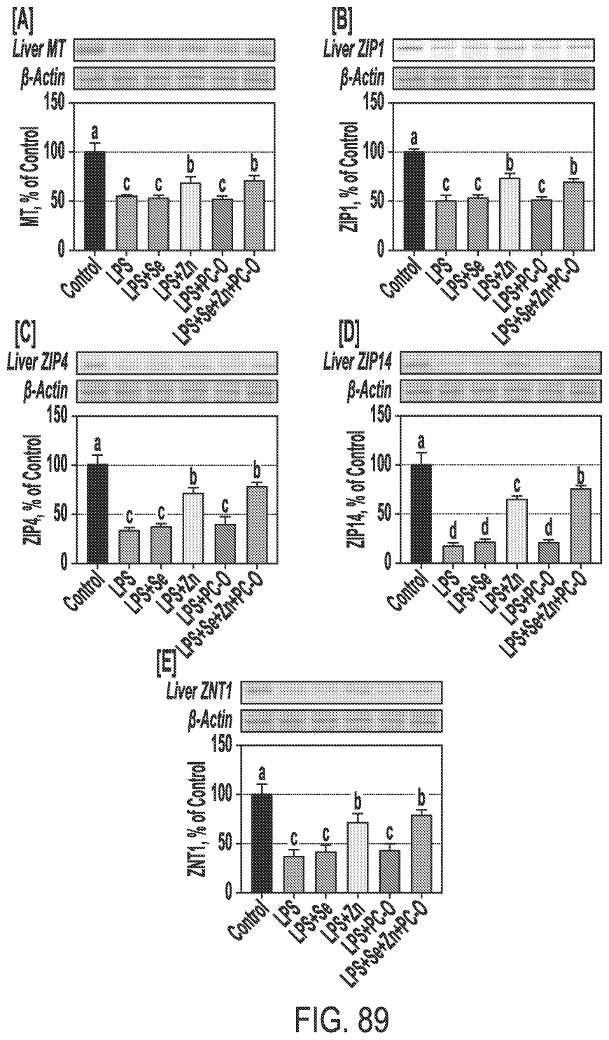

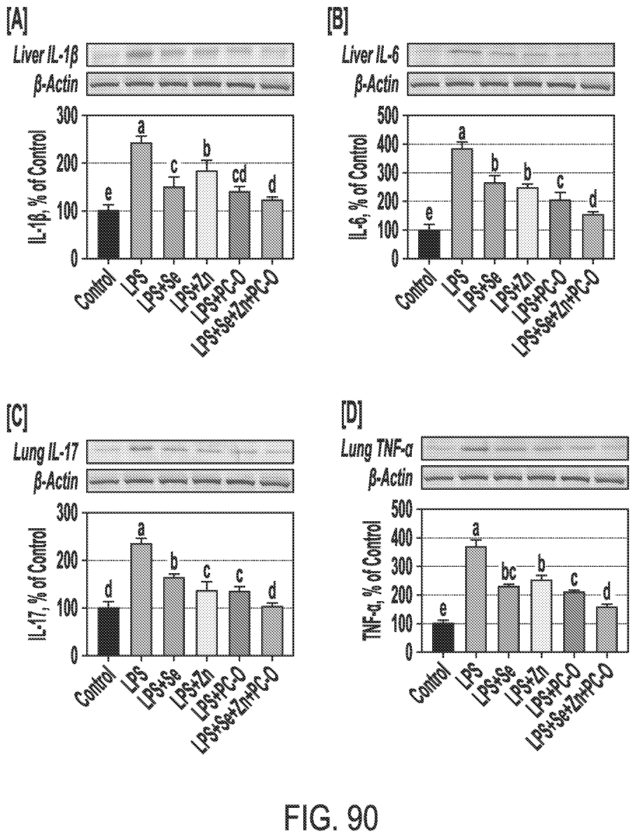

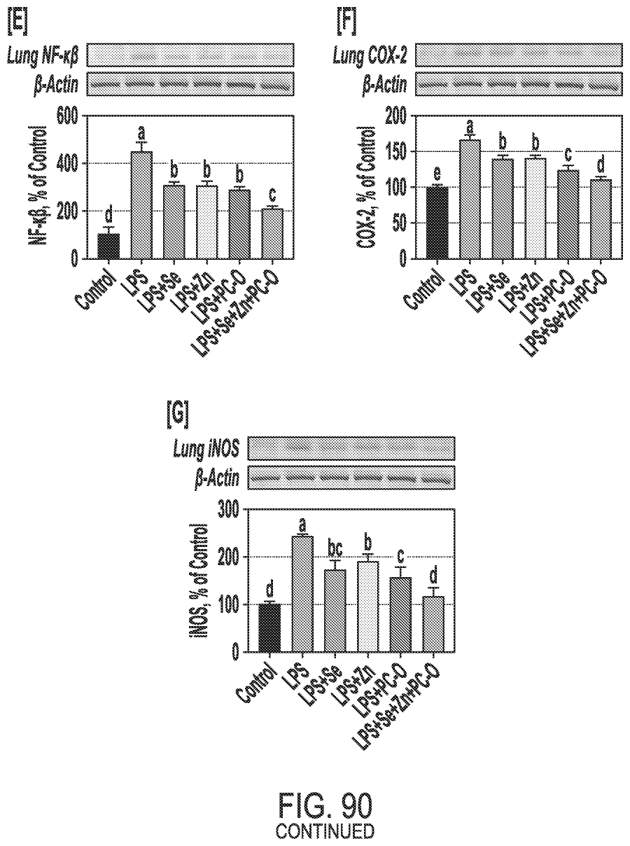

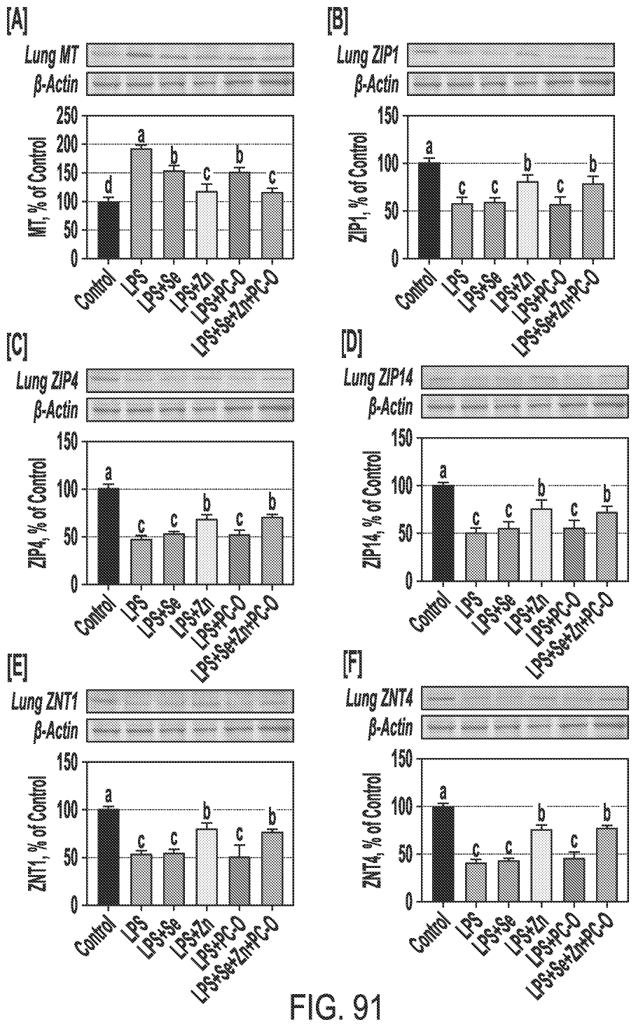

Effects of various dosages of Se, Zn, and O-CPs and their combination on LPS-induced febrile response and total pyrexia expressed as AUC of time course curves in mice are shown in FIG. 82B. FIG. 82A shows the effect of various dosages of Se, Zn, and O-CPs on rectal temperature of the subjects. In FIGS. 82A and 82B, the combination of Se, Zn and O-CPs reduces the total pyrexia in comparison with LPS control which shows higher pyrexia in absence of any treatment. Effects of various dosages of Se, Zn, O-CPs and their combination on serum ALT, AST, ALP and LDH levels in LPS induced pyrexia in mice are shown in FIGS. 83A-83D. Effects of various dosages of Se, Zn, O-CPs and their combination on serum interleukins (IL-6, IL-1β), TNF-α and serum NEU levels in LPS induced pyrexia in mice are shown in FIGS. 84A-84D. Effects of various dosages of Se, Zn, O-CPs and their combination on liver Zn, liver Se, lung Zn, and lung Se levels in LPS induced pyrexia in mice are shown in FIGS. 85A-85D, respectively. Effects of various dosages of Se, Zn, O-CPs and their combination on lung injury and on liver damage in LPS induced pyrexia in mice are shown in FIGS. 86-87, respectively. Effects of various dosages of Se, Zn, O-CPs and their combination on liver interleukins (IL-1β, IL-6, IL-17), TNF-α, NF-κβ, COX-2, and iNOS levels in LPS induced pyrexia in mice are shown in FIGS. 88A-88G, respectively. Effects of various dosages of Se, Zn, O-CPs and their combination on liver MT, ZIP1, ZIP4, ZIP14, and ZNT1 levels in LPS induced pyrexia in mice are shown in FIGS. 89A-89E, respectively. Effects of various dosages of Se, Zn, O-CPs and their combination on lung interleukins (IL-1β, IL-6, IL-17), TNF-α, NF-κβ, COX-2, and iNOS levels in LPS induced pyrexia in mice are shown in FIGS. 90A-90G. Effects of various dosages of Se, Zn, O-CPs and their combination on lung MT, ZIP1, ZIP4, ZIP14, ZNT1, and ZNT4 levels in LPS induced pyrexia in mice are shown in FIGS. 91A-91G, respectively.

### Example 26

Materials and Methods

O-CPs and PC are water-soluble. A 100 mg/mL stock solution was prepared in physiological saline. The powders were allowed to dissolve, and the solution was then sterile filtered through a 0.22 micron filter. Serial dilutions were made in physiological saline. EpiCor and Wellmune contain water-soluble compounds, but also contain insoluble solids. Aqueous extracts were prepared in physiological saline. The powders were allowed to re-hydrate for 1 hour under gentle agitation. The solids were precipitated by centrifugation, and the supernatants were then sterile filtered through a 0.22 micron filter. Serial dilutions were made in physiological saline. The solids remaining after centrifugation were re-suspended and washed once in physiological saline to remove or highly dilute any remaining aqueous compounds. Table 1 below shows the products tested.

TABLE 1
Products tested

| | Product | Solubility | Handling |
|---|---|---|---|
| 1. | Phycocyanin-O | Water-soluble | Aqueous |
| 2. | Phycocyanin | Water-soluble | Aqueous |
| 3. | EpiCor | Contains water-soluble compounds | Aqueous |
| 4. | Wellmune | Contains water-soluble compounds | Aqueous |

### Example 27

Tests Performed

Two different antioxidant tests were used to compare the test products. The Folin-Ciocalteu assay shows whether a given test product contains antioxidants. The CAP-e assay shows whether some of those antioxidants are bioavailable at the cellular level. The testing was performed twice for each assay: A first run to allow for an initial crude screening of effects across a standard dose range, and a second run that allowed fine-tuning of optimal doses for a good comparison of these specific products.

In addition, the effects of the test products on immune activation were measured using flow cytometry. The expression of the CD69 and CD25 activation markers were evaluated on Natural Killer cells, NKT cells, T cells, and monocytes/macrophages. The culture supernatants were used to test for production of cytokines, anti-viral peptides, and growth factors involved in regenerative functions. The following testing was conducted:

Immune Cell Activation:
(i) Direct effects of the test products on immune cell activation status.
(ii) Effects when immune cells are pre-treated with the test products immediately prior to an inflammatory challenge with the bacterial toxin LPS.
(ii) Effects when immune cells are pre-treated with the test products immediately prior to a viral mimetic challenge, using Poly I:C.

For Each of the 3 Immune Cell Culture Conditions, the Following were Tested:
(i) Induction of the 2 activation markers CD69 and CD25.
(ii) Cytokine production.

Test for Total Antioxidant Capacity

The products were tested for antioxidant capacity in the Folin-Ciocalteu assay (also known as the total phenolics assay). This assay made use of the Folin-Ciocalteu reagent to measure antioxidants. The assay was performed by adding the Folin-Ciocalteu's phenol reagent to serial dilutions of extract, thoroughly mixing, and incubating for 5 minutes. Sodium carbonate was added, starting a chemical reaction producing a color. The reaction was allowed to continue for 30 minutes at 37° C. Optical absorbance was measured at 765 nm in a colorimetric plate reader. Gallic acid was used as a reference standard, and the data reported in Gallic Acid Equivalents per gram product.

Cell-Based Antioxidant Protection Assay

This allows assessment of antioxidant potential in a method that is comparable to the ORAC test, but only allows measurement of antioxidants that are able to cross the lipid bilayer cell membrane, enter the cells, and provide biologically meaningful antioxidant protection under conditions of oxidative stress. The CAP-e bioassay was specifically developed to work with natural products and ingredients. This method has been used on multiple types of natural products and ingredients.

As a model cell type, the red blood cells (RBCs) were used. This was an inert cell type, in contrast to other cell types such as polymorphonuclear (PMN) cells (often used for subsequent testing of anti-inflammatory effects of natural product and extracts). The red blood cell-based assay was developed particularly to be able to assess antioxidants from complex natural products in a cell-based system and help interpret subsequent data from more complex cellular models.

During the incubation with a test product, any antioxidant compounds able to cross the cell membrane can enter the interior of the RBC. Then the RBC were washed to remove compounds that were not absorbed by the cells, and loaded with the dye, such as, DCF-DA dye, which turns fluorescent upon exposure to reactive oxygen species. Oxidation was triggered by addition of the peroxyl free radical generator AAPH. The fluorescence intensity was evaluated. The low fluorescence intensity of untreated control cells served as a baseline, and RBC treated with AAPH alone served as a positive control for maximum oxidative damage.

If a reduced fluorescence intensity was observed, of RBC exposed to a test product and subsequently exposed to AAPH, this indicated that the test product contained antioxidants available to penetrate into the cells and protect these from oxidative damage. Based on the low fluorescence of the untreated control wells, and the high fluorescence of the cell cultures exposed to oxidative damage, the fluorescence intensity in cell cultures treated with test products prior to exposure to oxidative stress was used to calculate the percent inhibition of cellular oxidative stress.

Oxygen Radical Absorbance Capacity (ORAC) Tests

ORAC tests are among the most acknowledged methods that measure antioxidant scavenging activity against oxygen radicals that are known to be involved in the pathogenesis of aging and many common diseases. ORAC 6.0 consists of six types of ORAC assays that evaluate the antioxidant capacity of a material against six primary reactive oxygen species (ROSs, commonly called “oxygen radicals”) found in humans: peroxyl radical, hydroxyl radical, superoxide anion, singlet oxygen, peroxynitrite, and hypochlorite. This was a comprehensive panel that evaluated the antioxidant capacity of a material against oxygen radicals.

The ORAC 6.0 tests were based on evaluating the capacity of an interested material to protect a probe (a fluorescent probe or chromagen) from its damage by ROSs. In all ORAC assays, an ROS inducer was introduced to the assay system. The ROS inducer triggered the release of a specific ROS, which would degrade the probe and cause its emission wavelength or intensity change. When an antioxidant material present in the environment, the antioxidant absorbs the ROS and preserved the probe from degradation. The degree of probe preservation indicated the antioxidant capacity of the material. Trolox was used as the reference standard, and the results were expressed as μmol Trolox equivalency per gram (or milliliter) of a tested material.

Cell Survival or Viability—Preparation for Further Bioassay Work

For the particular purpose of testing the activating effects of the test products on immune cell activation and cytokine production, a cell viability assay was needed as a preparatory step when starting work on the biological effects of complex natural products. The data generated from this testing helped identify the optimal dose range for the immune cell testing. Peripheral blood mononuclear cells were tested for viability as reflected by mitochondrial function using the MTT assay. The MTT assay utilizes a dye that changes color dependent on mitochondrial activity. Freshly harvested cells were cultured to allow the color formation to take place in proportion to mitochondrial function. In the MTT bioassay, chemical reactions trigger a specific color development based on cellular functions:
When a reduction in color was measured, this was linked to a reduced cellular viability, either as a result of direct killing, or inhibition of mitochondrial function.
When an increase in color was measured, this had several possible explanations: 1) increased cell numbers (growth); 2) increased mitochondrial mass, and 3) increased mitochondrial function (energy production).

Seven doses were tested, using 2-fold dilutions in the assay. The dose range for the 4 products, namely, O-CPs, PC, EpiCor, and Wellmune was 0.039 g/L-2.5 g/L.

Effect on Immune Modulation in Context of Bacterial and Viral Challenges

Human peripheral blood mononuclear cells (PBMC) blood cultures were used for this testing. Three sets of parallel cultures were established, using PBMC from a healthy human blood donor. The highly inflammatory bacterial LPS from E. coli was used as a positive control for activation and was also used in one of the three cultures to induce inflammation after treating the immune cells with a natural product. The viral-mimic Polyinosinic: polycytidylic acid (Poly I:C) was used in the third set of cultures. The testing was performed such that all treatments, including each dose of test product and each positive and negative control, were tested in triplicate.

The three parallel culture conditions were:
(i) adding serial dilutions of the test product to PBMC cultures in the absence of an inflammatory stimulus, in order to study the direct effect of the test product;
(ii) adding serial dilutions of the test product to PBMC cultures, allowing a ‘priming’ of the immune cells, and subsequently add the inflammatory stimulus LPS; and
(iii) adding serial dilutions of the test product to PBMC cultures, allowing a ‘priming’ of the immune cells, and subsequently add the stimulus Poly I:C.
The PBMC cultures were incubated for 24 hours, after which the cells and the culture supernatants were harvested and used to monitor the reactions in each culture.

Expression of Activation Markers

While CD69 plays an important role in immunity through the increase of lymphocyte proliferation and cellular signaling, CD69 has recently been implicated in the immunomodulatory effects leading to the control of inflammation. CD69 was rapidly induced in NK cells shortly after activation, and its direct role in NK cytotoxicity had been demonstrated. The contents of Borrego F et al., Regulation of CD69 Expression on Human Natural Killer Cells: Differential Involvement of Protein Kinase C and Protein Tyrosine Kinases; and Moretta A et al., CD69-mediated pathway of lymphocyte activation: anti-CD69 monoclonal antibodies trigger the cytolytic activity of different lymphoid effector cells with the exception of cytolytic T lymphocytes expressing T cell receptor alpha/beta, are hereby incorporated by reference. When human NK cells were co-cultured with K562 target cells, CD69 expression was upregulated, and the increase significantly correlated with NK cell activity, as measured by today's gold standard CD107 mobilization assay. The contents of Dons'koi B V et al., Measurement of NK activity in whole blood by the CD69 up-regulation after co-incubation with K562, comparison with NK cytotoxicity assays and CD107a degranulation assay, are hereby incorporated by reference. CD69 had the capacity to activate the NK cytolytic machinery in the absence of other NK-target cell adhesion molecule interactions. The contents of Borrego F et al., CD69 is a stimulatory receptor for natural killer cell and its cytotoxic effect is blocked by CD94 inhibitory receptor, are hereby incorporated by reference. Importantly: A direct and highly significant correlation between CD69 levels and NK cell activity was demonstrated by Clausen et al. 2003, in a study involving 14 breast cancer patients tested repeatedly during chemotherapy. The contents of Clausen J et al., Functional significance of the activation-associated receptors CD25 and CD69 on human NK-cells and NK-like T-cells, are hereby incorporated by reference. Therefore, in immunomodulating of natural products, CD69 staining for NK cell activation (and indicative of NK cell activity) has been used, both in-vitro and in clinical studies.

CD25 is the receptor for the cytokine IL-2 and is present on activated T cells and B cells. It was shown that in some situations NK cells decide whether to enter a mode of proliferation with predominant expression of CD25, or to enter into a highly cytotoxic killing mode, in which case they preferentially express CD69.

### Example 28

### Effects on Total Antioxidant Capacity

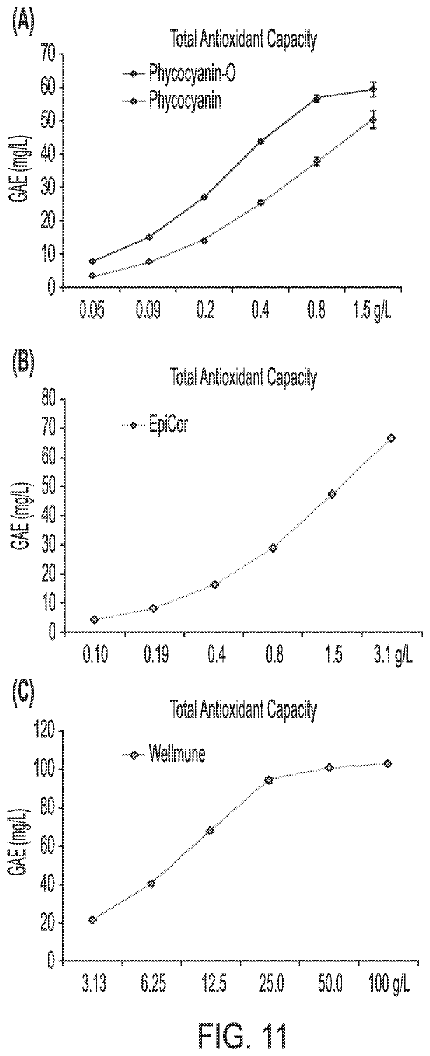

O-CPs (Phycocyanin-O) had the strongest total antioxidant capacity, stronger than PC and EpiCor. Wellmune had little total antioxidant capacity. FIGS. 11A-11C shows the total antioxidant capacity of O-CPs, PC, EpiCor, and Wellmune, respectively. Table 2 shows that on a per-weight basis O-CPs had a 3-fold stronger total antioxidant capacity than PC. EpiCor had a slightly lower total antioxidant capacity than PC. Wellmune had low total antioxidant capacity.

TABLE 2
Comparison of cellular antioxidant capacity

| Name | GAE (mg/L) |
|---|---|
| Phycocyanin-O | 117 |
| Phycocyanin | 34 |
| EpiCor | 22 |
| Wellmune | 5 |

### Example 29

Oxygen Radical Absorbance Capacity (ORAC)

There are six predominant reactive species found in the body: peroxyl radicals, hydroxyl radicals, peroxynitrite, super oxide anion, singlet oxygen and hypochlorite. ORAC 6.0 provided comprehensive analyses of antioxidant capacity of a food/nutrition product against the six predominant reactive species. The ORAC result was expressed as micromole Trolox equivalency (μmol TE) per gram. Tables 4, 5 and 6 show the antioxidative effect of O-CPs powder, 14032019), PC food grade powder (powder, CU180), and Spirulina super Blu powder (powder, 401G174098), respectively. It was observed that O-CPs showed the best results in terms of antioxidative effect as compared to PC food grade powder and Spirulina super Blu powder. The greater the number, the greater the antioxidative effect is.

TABLE 4
ORAC results of O-CPs (powder, 14032019)

| Analysis | Result | Units |
|---|---|---|
| ORAC against peroxyl radicals | 476.87 | μmole TE/gram |
| ORAC against hydroxyl radicals | 733.64 | μmole TE/gram |
| ORAC against peroxynitrite | 40.29 | μmole TE/gram |
| ORAC against super oxide anion | 172.06 | μmole TE/gram |
| ORAC against singlet oxygen | 93.23 | μmole TE/gram |
| ORAC against hypochlorite | 2,804.52 | μmole TE/gram |

TABLE 5
ORAC results of PC food grade powder (powder, CU180)

| Analysis | Result | Units |
|---|---|---|
| ORAC against peroxyl radicals | 25.64 | μmole TE/gram |
| ORAC against hydroxyl radicals | 62.31 | μmole TE/gram |
| ORAC against peroxynitrite | 6.70 | μmole TE/gram |
| ORAC against super oxide anion | 53.15 | μmole TE/gram |
| ORAC against singlet oxygen | 70.18 | μmole TE/gram |
| ORAC against hypochlorite | 311.68 | μmole TE/gram |

TABLE 6
ORAC results of Spirulina super
Blu powder (powder, 401G174098)

| Analysis | Result | Units |
|---|---|---|
| ORAC against peroxyl radicals | 20.32 | μmole TE/gram |
| ORAC against hydroxyl radicals | 41.47 | μmole TE/gram |
| ORAC against peroxynitrite | 0.65 | μmole TE/gram |
| ORAC against super oxide anion | Not Detected | μmole TE/gram |
| ORAC against singlet oxygen | 105.83 | μmole TE/gram |
| ORAC against hypochlorite | Not Detected | μmole TE/gram |

### Example 30

Cell Survival/Viability

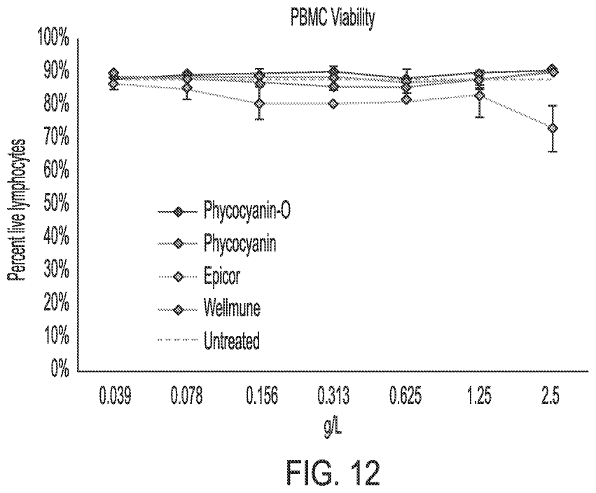

The PBMC cells tolerated 3 of the test products very well: O-CPs, PC, and Wellmune. The cells were mildly stressed by the highest dose of EpiCor (2.5 g/L). Based on this data the dose range for the immune activation testing for the 4 algae- and yeast-based products was chosen as 0.25-2.0 g/L. In FIG. 12, the effects of various dosages of O-CPs, PC, EpiCor, and Wellmune on percent live lymphocytes are shown.

### Example 31

Effects on Immune Activation and Modulation in Context of Direct Effects, Bacterial and Viral Challenges

The PBMC cultures were incubated for 24 hours, after which the cells and the culture supernatants were harvested and used to monitor the reactions in each culture. The cells were stained with a combination of monoclonal antibodies to monitor activation, and analyzed by multi-parameter flow cytometry, using an acoustic dual laser Attune flow cytometer. The analysis included fluorescent markers for CD3, CD25, CD56, and CD69. This combination allowed monitoring of changes to monocyte/macrophages, as well as activation of natural killer cells, NKT cells, and T lymphocytes. The staining with CD3 and CD56 allowed to identify CD3-CD56+NK cells, CD3+CD56− T lymphocytes, and CD3+CD56+NK-T cells. Upon combining this with forward/side scatter analysis for cellular size and granularity, the monocyte population can also be identified. For each of these populations the expression level of the activation markers CD25 and CD69 can be examined.

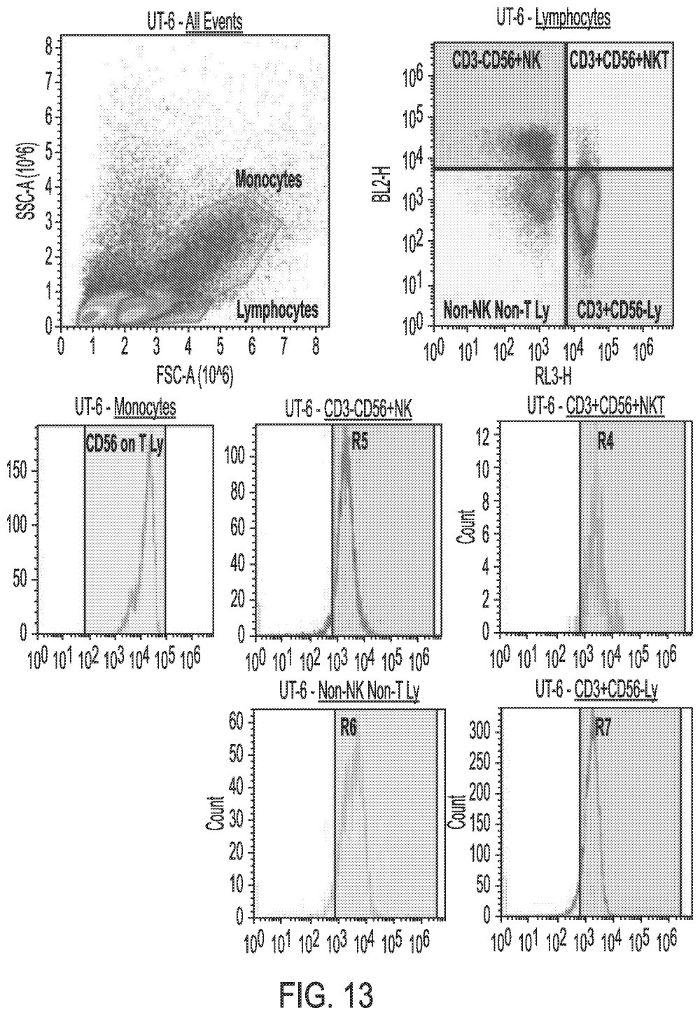

During data analysis, the physical properties of different cell types allow electronic gating on lymphocytes versus monocytes, so that the CD69 versus CD25 expression can be analyzed on these cell types separately. In addition, the lymphocyte fraction was divided into 4 separate subpopulations, based on whether cells stain with CD3, CD56, or both. In FIG. 13, the flow cytometry data showed gates for lymphocytes, monocytes, and the four subsets of lymphocytes, allowing analysis of CD69 expression on all five cell types.

Immune Activation

Direct immune cell activation, as well as immune modulation in context of bacterial and viral challenges, were monitored by expression levels of CD69 and CD25:
O-CPs showed a robust effect on immune cell activation.
EpiCor had some direct immune cell-activating effects, and Wellmune had almost none.

Algae-Based and Fermentation-Based Products: Effects on Cytokines:

Culture supernatants were used to test for a panel of 7 immune-activating cytokines, 2 anti-inflammatory cytokines, and a growth factor, G-CSF, involved in stem cell regulation and reparative functions. The cytokine results showed interesting differentiation between O-CPs and PC. O-CPs had unique and strong effects on cytokines than PC:
O-CPs triggered higher levels of IL-6, both under unstressed culture conditions and in context of the viral challenge.
O-CPs directly triggered higher levels of MIP-1β in the absence of an immune challenge than PC.
In context of the bacterial or viral immune challenge, O-CPs triggered increased levels of MIP-1β, in stark contrast to PC that strongly reduced MIP-1β levels.
In context of an immune challenge, O-CPs triggered higher levels of the anti-inflammatory cytokine IL-1ra compared to PC.
O-CPs triggered lower levels of IL-1β, TNF-α, and IFN-γ than PC.
O-CPs induced the growth factor G-CSF.

In the absence of an immune challenge, EpiCor and Wellmune had fewer effects than PC, and for the most part also less effects than O-CPs. In context of a bacterial immune challenge, EpiCor, and to some extent Wellmune, triggered higher levels of IL-6, IL-8, MIP-1α, and MIP-1β than the O-CPs and PC.

### Example 32

Immune Activation by Algae-Based and Fermentation-Based Products

Direct Effects of Test Products on Immune Cell Activation

Comparing O-CPs to PC: Effects on CD69 Levels:

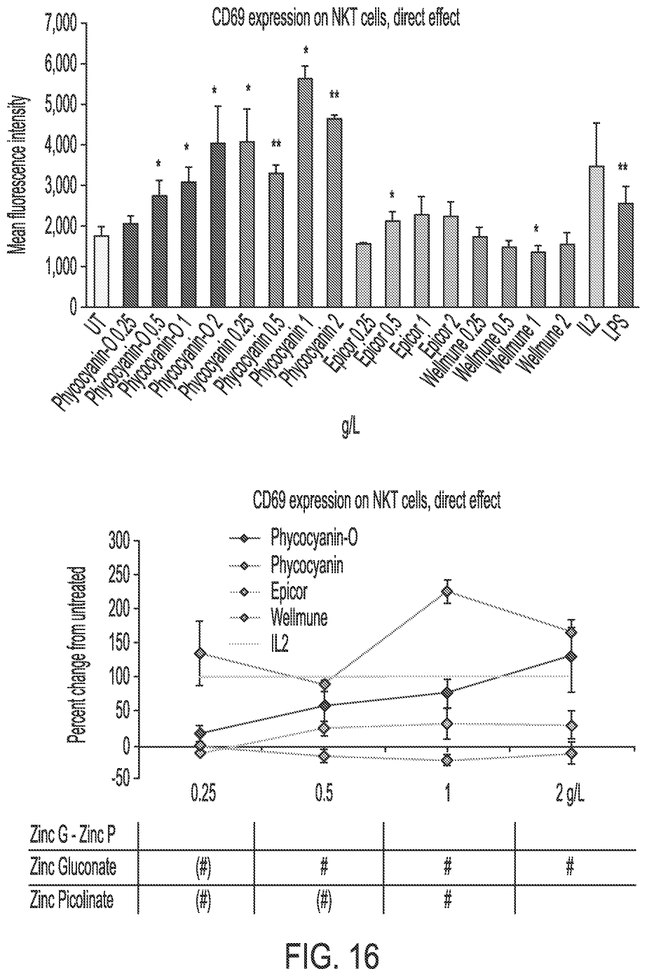

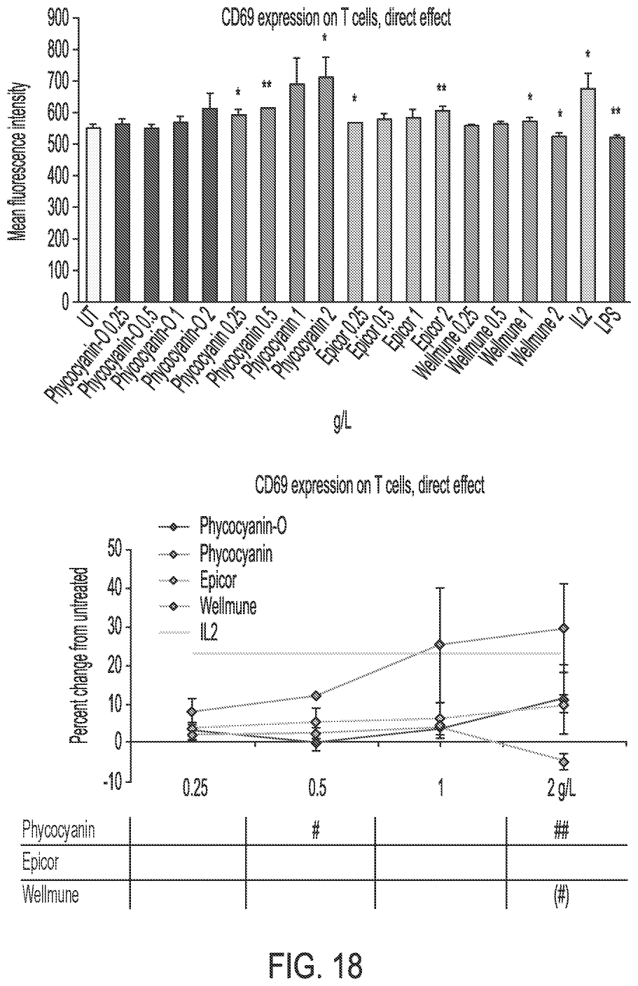

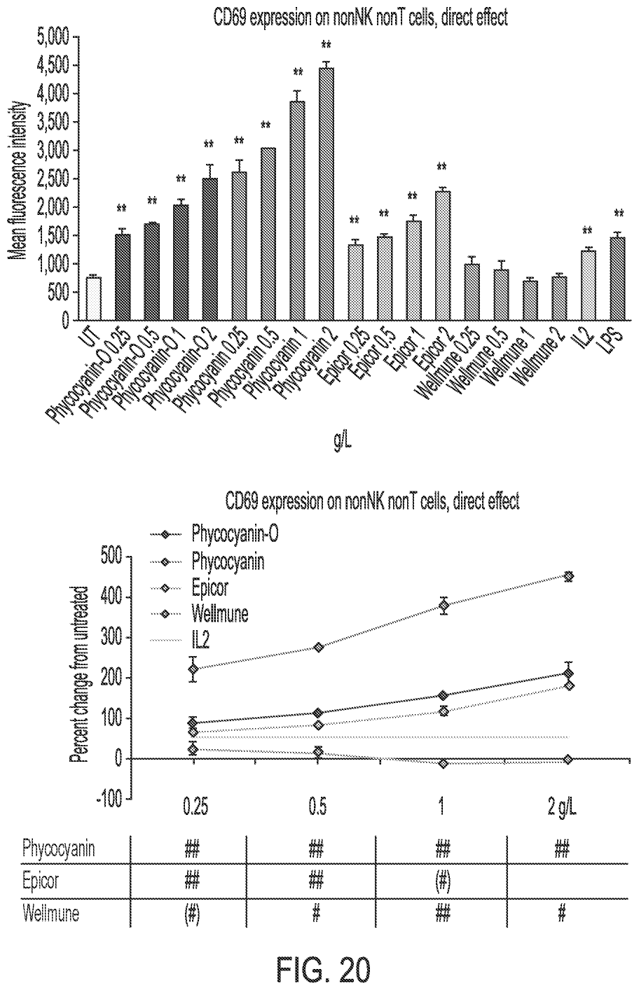

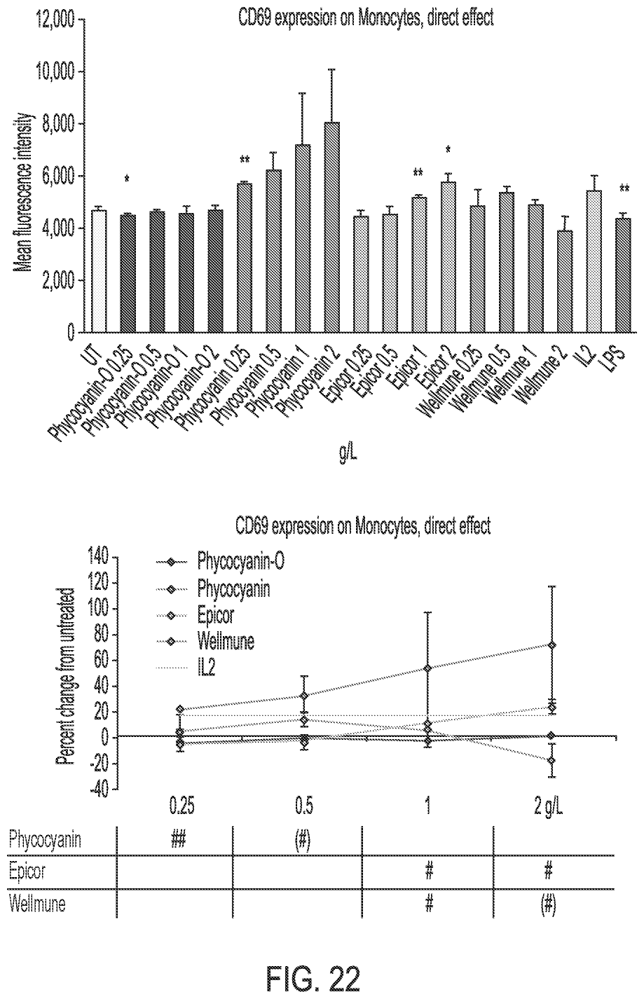

O-CPs directly triggered upregulation of CD69 on all 4 types of lymphocytes in the absence of an immune challenge, while not affecting CD69 expression monocytes. These results relating to the expression of CD69 on NK cells is shown in FIG. 14. Expression of CD69 on NKT cells is shown in FIG. 16; expression of CD69 on T cells is shown in FIG. 18; and the expression of CD69 on non-NKT non-T cells is shown in FIG. 20. Expression of CD69 on monocytes is shown in FIG. 22.

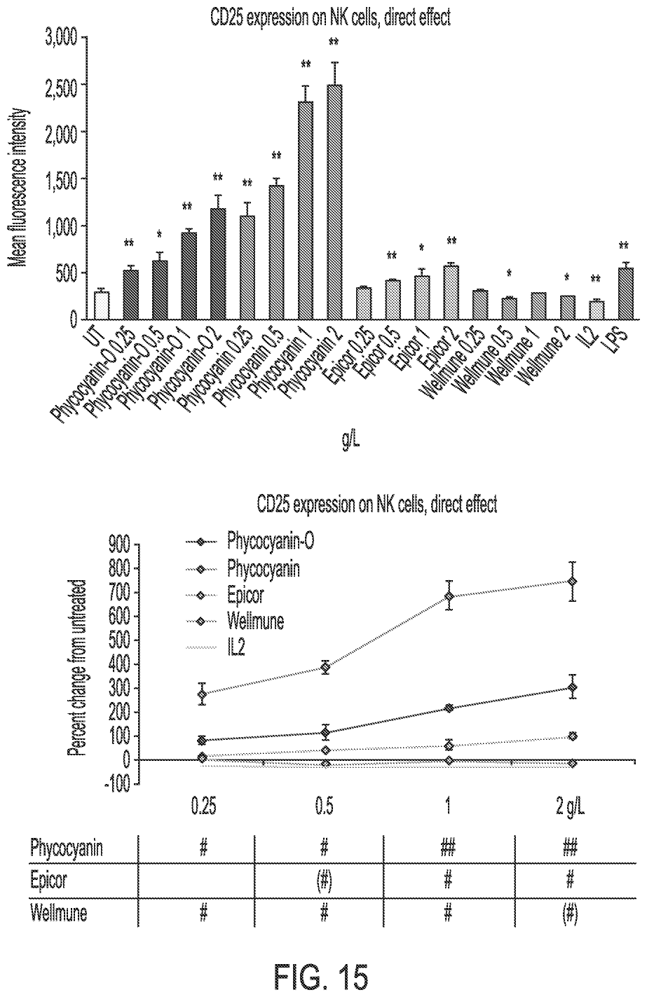

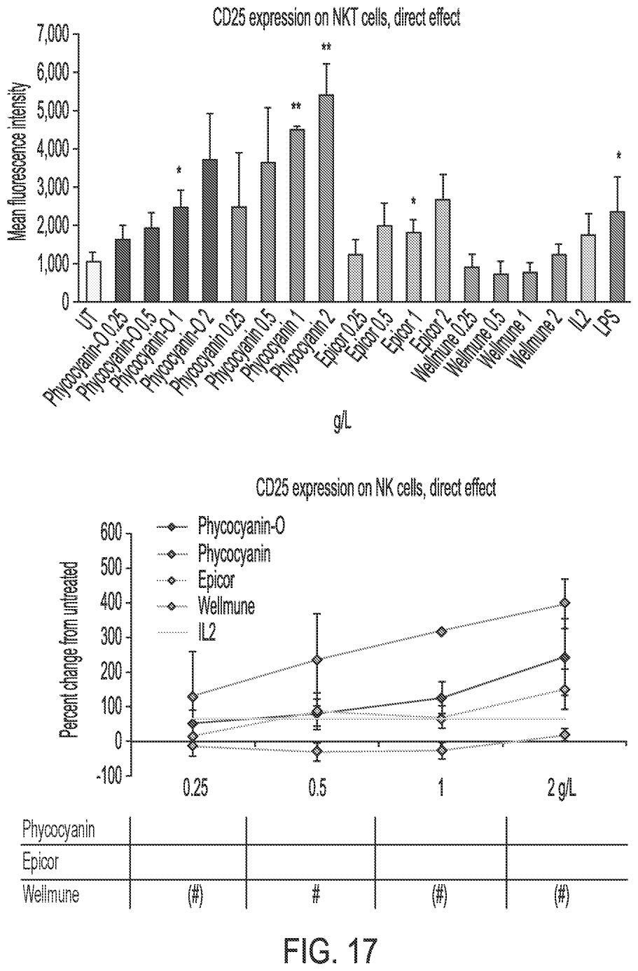

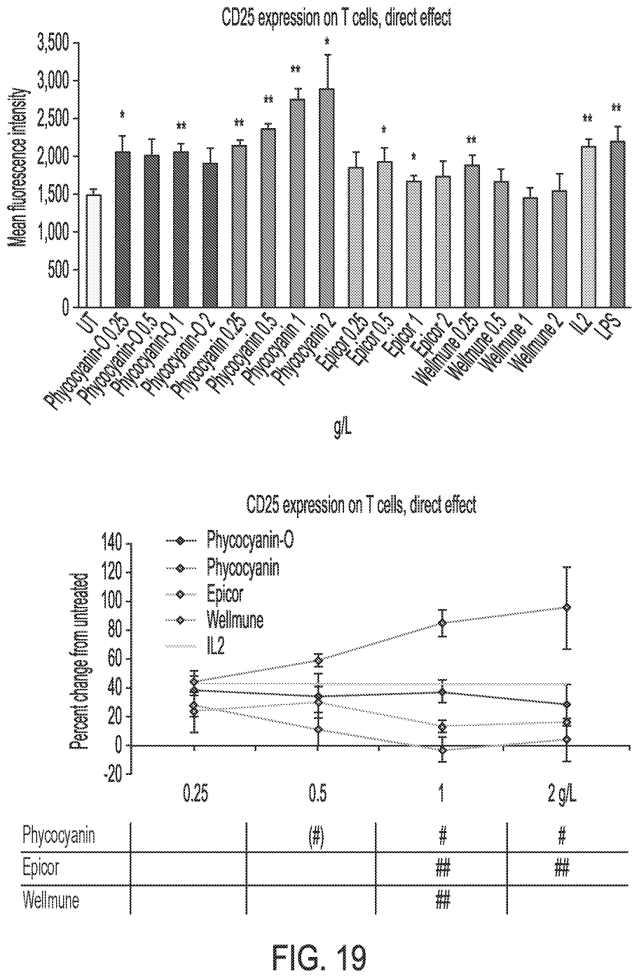

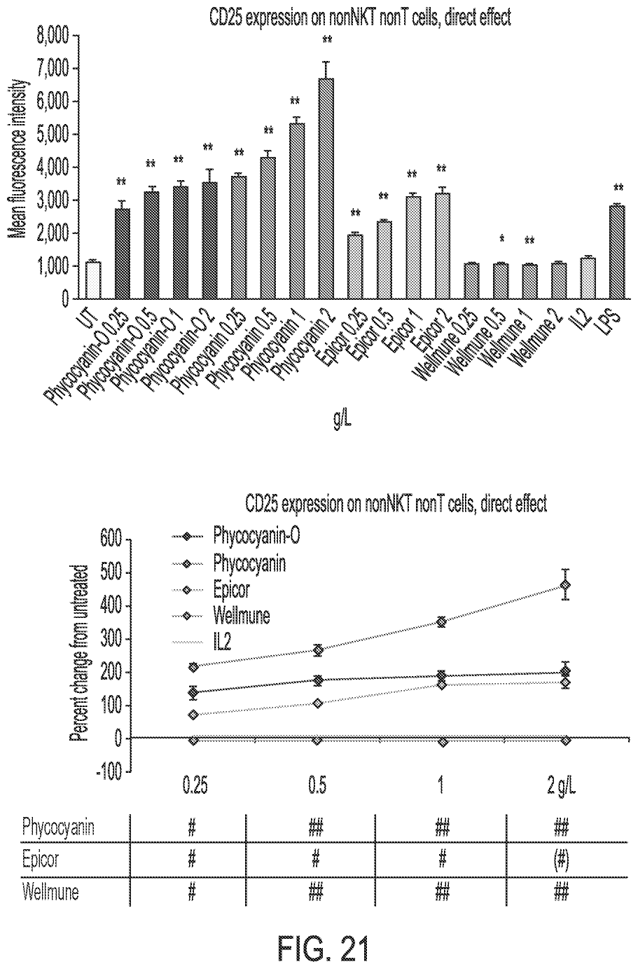

Effects on CD25 levels: O-CPs directly triggered upregulation of CD25 on all 4 types of lymphocytes in the absence of an immune challenge. These results relating to the expression of CD25 on NK cells are shown in FIG. 15. Expression of CD25 on NKT cells are shown in FIG. 17; expression of CD25 on T cells are shown FIG. 19; and the expression of CD25 on non-NKT non-T cells are shown in FIG. 21.

Comparing O-CPs to EpiCor and Wellmune (aqueous fraction): Effects on CD69 Levels:

In the absence of an immune challenge, direct effects of the test products were evaluated in which O-CPs and EpiCor showed comparable mild effects, whereas Wellmune had no direct effect. These results are shown in FIGS. 14, 16, 18, 20, 22.

Effects on CD25 Levels:
NK cells: In the absence of an immune challenge, direct effects of the test products were evaluated wherein O-CPs strongly increased CD25 expression, EpiCor showed a much milder direct effect, and Wellmune had no direct effect, as shown in FIG. 15.
NKT cells: In the absence of an immune challenge, direct effects of the test products were evaluated wherein O-CPs increased CD25 expression, neither EpiCor nor Wellmune showed a direct effect on CD25 expression, as shown in FIG. 17.
T lymphocytes: None of the 3 test products had a direct effect on CD25 expression, as shown in FIG. 19.
Non-NK non-T cells: while O-CPs strongly increased CD25 expression, EpiCor showed a slightly milder direct effect, and Wellmune had no direct effect, as shown in FIG. 21.

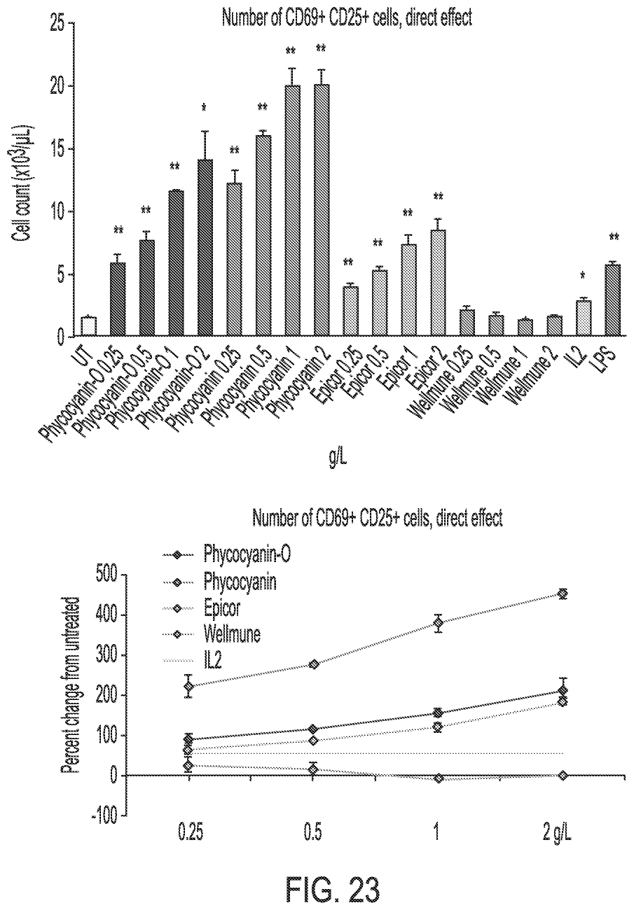

Effects on CD69+CD25+ cells: In the absence of an immune challenge, O-CPs stimulated an increase in CD69+CD25+ double-positive cells. EpiCor had a milder effect, and Wellmune had no effect on the number of CD69+CD25+ cells, as shown in FIG. 23.

### Example 33

Modulation of Immune Response Under LPS-Induced Inflammation

Comparing O-CPs to PC: Effects on CD69 Levels:

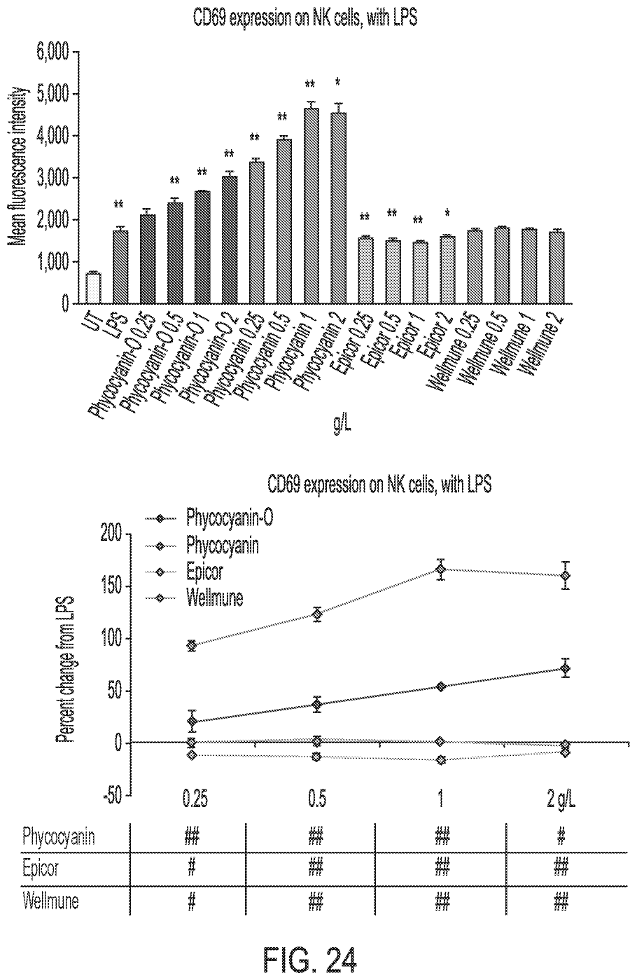

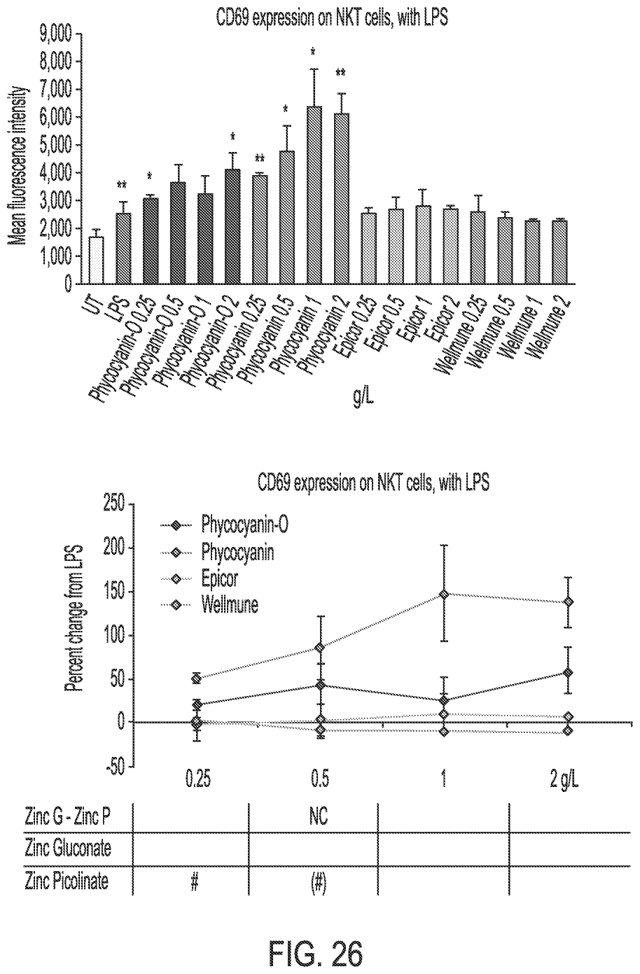

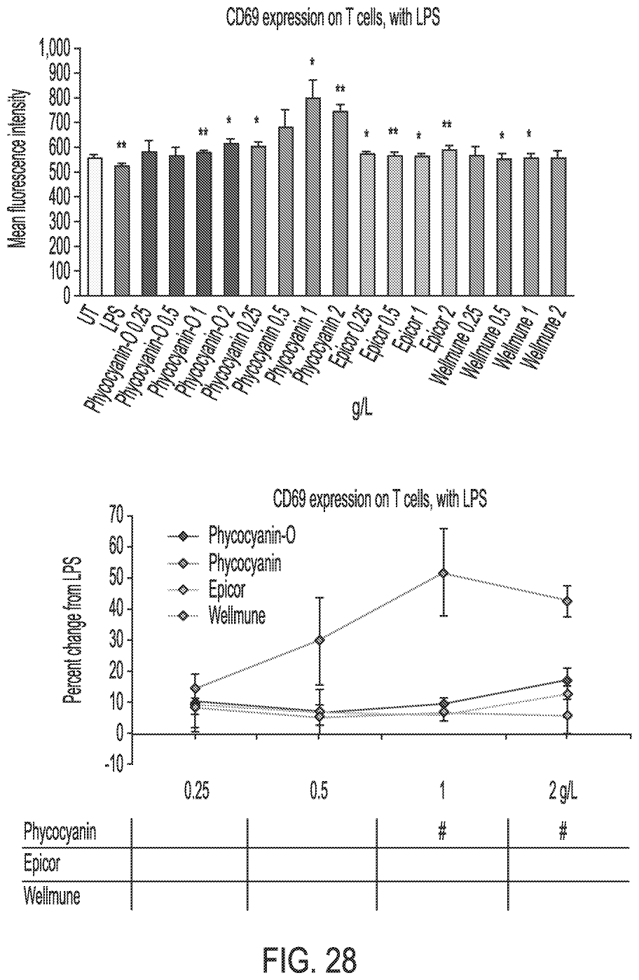

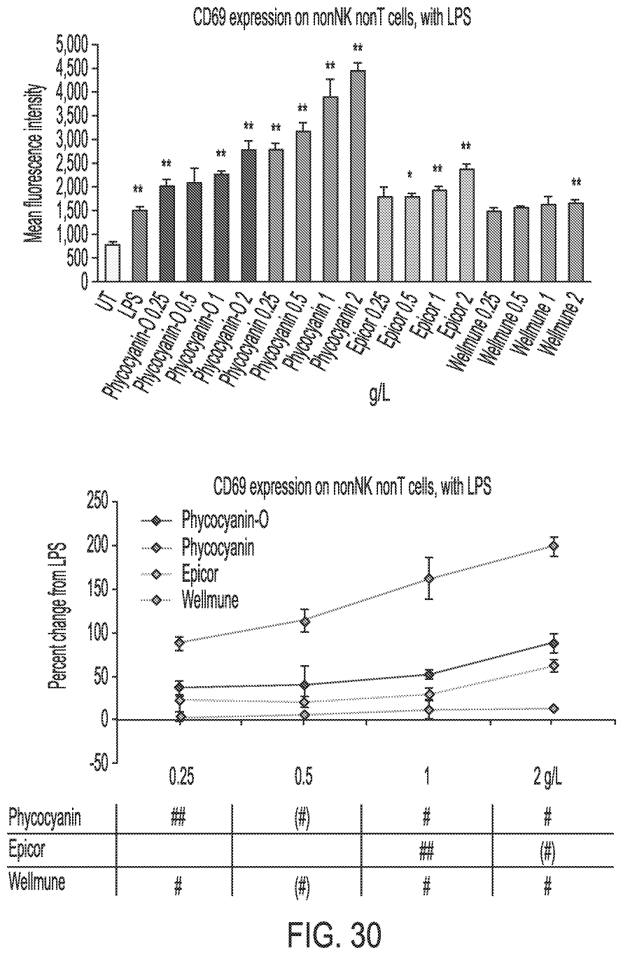

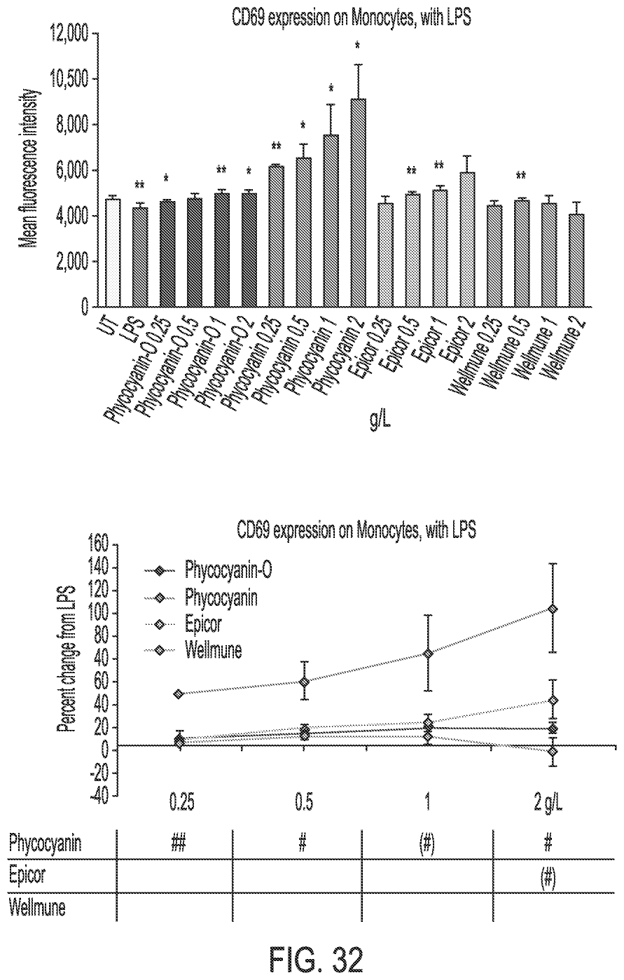

In context of an inflammatory immune challenge with the bacterial toxin LPS, the LPS-mediated increase in CD69 expression was further enhanced by O-CPs. This was seen for all cell types analyzed. The results relating to the expression of CD69 on NK cells is shown in FIG. 24. Expression of CD69 on NKT cells is shown in FIG. 26; expression of CD69 on T cells is shown in FIG. 28; and the expression of CD69 on non-NKT non-T cells is shown in FIG. 30. Monocytes: In the context of an LPS-mediated immune challenge, O-CPs enhanced the CD69 expression. Expression of CD69 on monocytes is shown in FIG. 32.

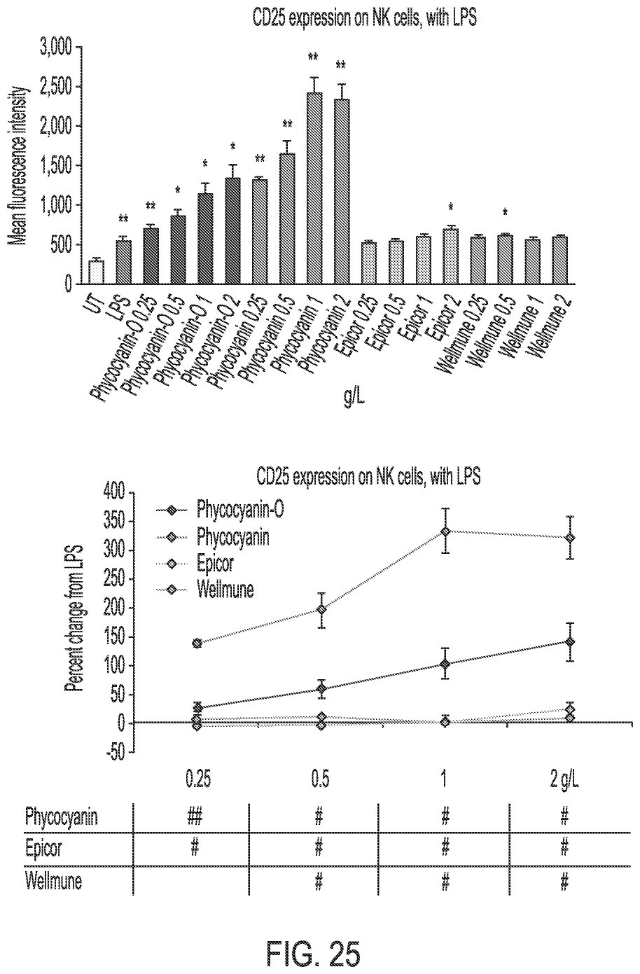

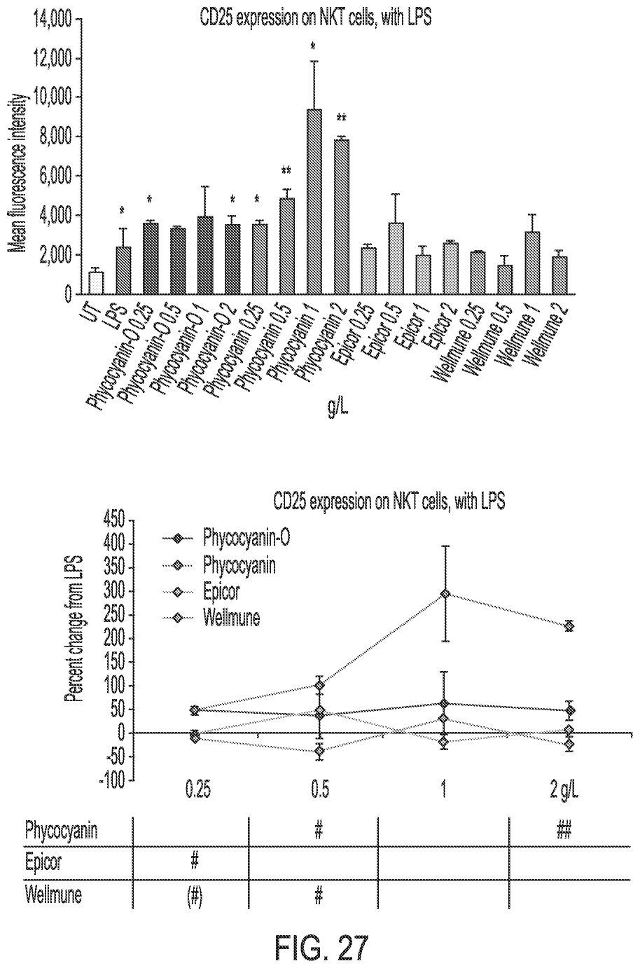

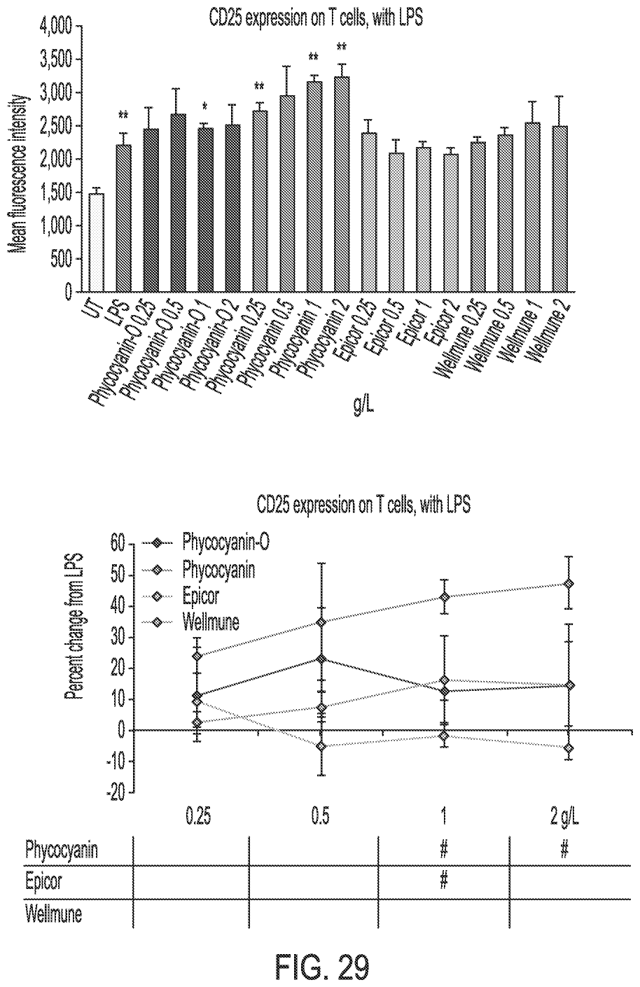

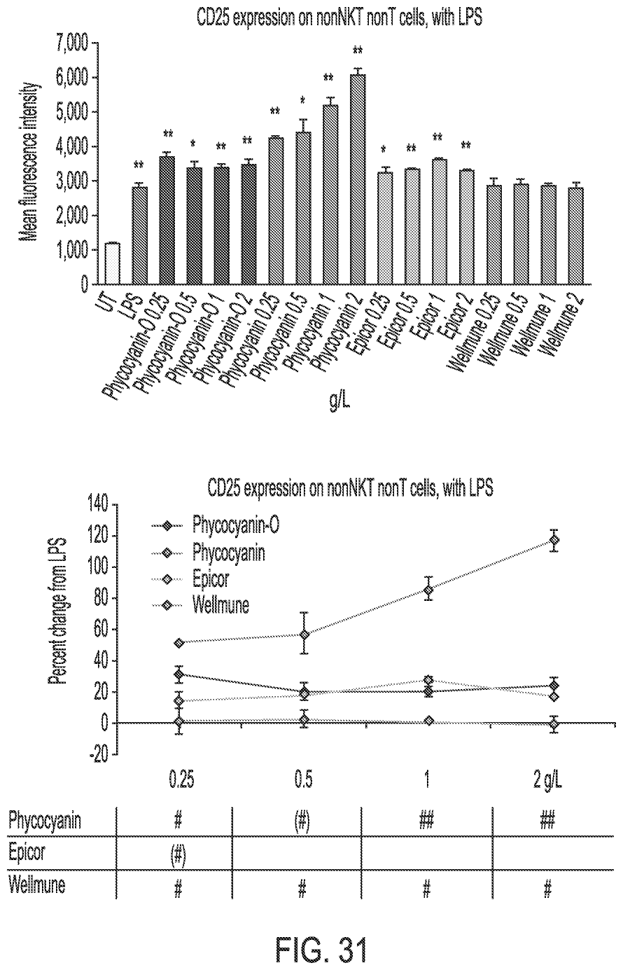

Effects on CD25 levels: In context of an inflammatory immune challenge with the bacterial toxin LPS, the LPS-mediated increase in CD25 expression was further enhanced by both O-CPs. This was seen on all 4 types of lymphocytes analyzed. These results relating to the expression of CD25 on NK cells are shown in FIG. 25. Expression of CD25 on NKT cells are shown in FIG. 27; expression of CD25 on T cells are shown in FIG. 29; and the expression of CD25 on non-NKT non-T cells are shown in FIG. 31.

Comparing O-CPs to EpiCor and Wellmune (Aqueous Fraction): Effects on CD69 Levels:

In context of an inflammatory immune challenge with the bacterial toxin LPS, the LPS-mediated increase in CD69 expression was further enhanced by O-CPs, but both EpiCor and Wellmune showed mostly different effects:
NK cells: EpiCor mildly reduced the LPS-mediated increase in CD69 expression. Wellmune had no effect, as shown in FIG. 24.
NKT cells: Neither EpiCor nor Wellmune altered the LPS-induced CD69 expression, as shown in FIG. 26.
T lymphocytes: This was an exception, since all 3 test products showed comparable, mild enhancement of LPS-mediated increased CD69 expression, as shown in FIG. 28.
Non-NK non-T cells: O-CPs increased CD69 expression more than EpiCor, and much more than Wellmune, as shown in FIG. 30.
Monocytes: EpiCor triggered a slightly higher increase in CD69 expression than O-CPs. Wellmune had barely any effect, as shown in FIG. 32.

Effects on CD25 Levels:

In context of an inflammatory immune challenge with the bacterial toxin LPS, the LPS-mediated increase in CD25 expression was modulated differently by O-CPs, EpiCor, versus Wellmune:
NK cells: O-CPs increased LPS-induced CD25 expression, EpiCor showed a much milder effect. Wellmune had no effect, as shown in FIG. 25.
NKT cells: O-CPs increased LPS-induced CD25 expression. Neither EpiCor nor Wellmune had an effect, as shown in FIG. 27.
T lymphocytes: None of the 3 test products showed major effects on LPS-mediated increased CD25 expression, as shown in FIG. 29.
Non-NK non-T cells: O-CPs and EpiCor triggered similar mild increases in CD25 expression whereas Wellmune had no effect, as shown in FIG. 31.

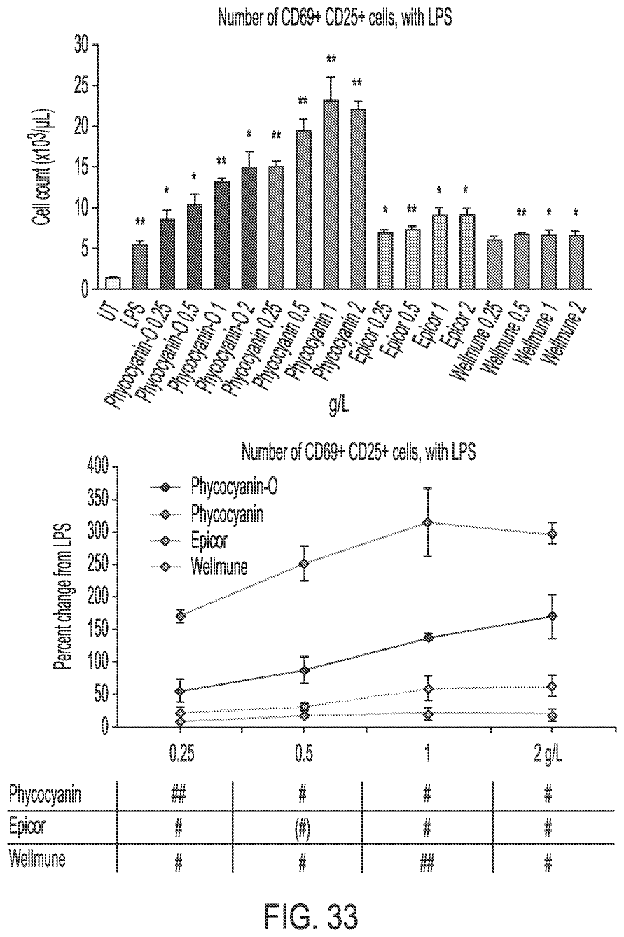

Effects on CD69+CD25+ cells: In the context of a bacterial inflammatory immune challenge, O-CPs stimulated an increase in CD69+CD25+ double-positive cells. EpiCor had a milder effect, and Wellmune had barely any effect on the number of CD69+CD25+ cells, as shown in FIG. 33.

### Example 34

Modulation of Immune Response to a Viral Mimetic Poly I:C

Comparing O-CPs to PC: Effects on CD69 Levels:

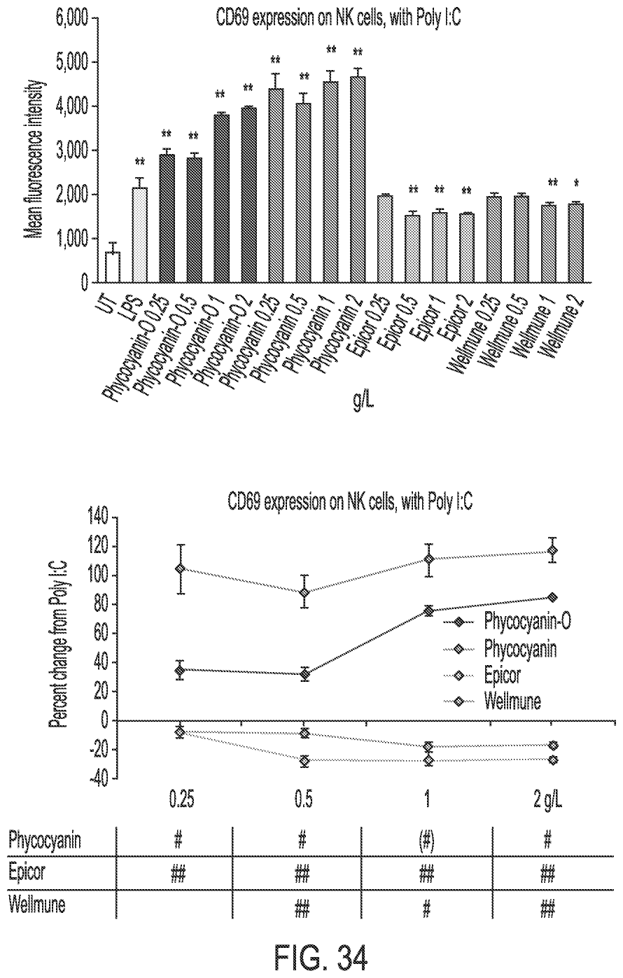

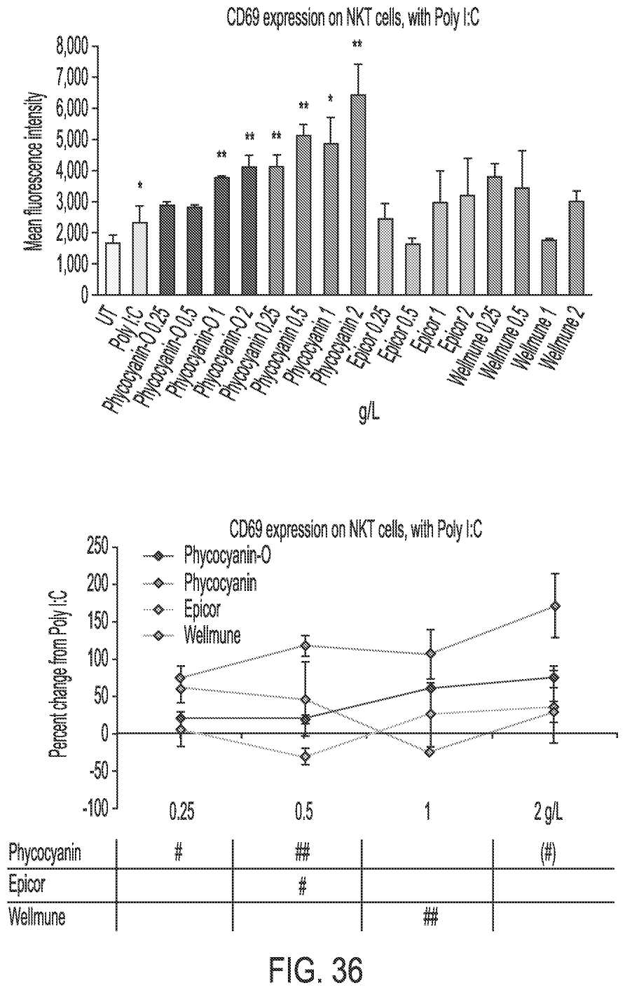

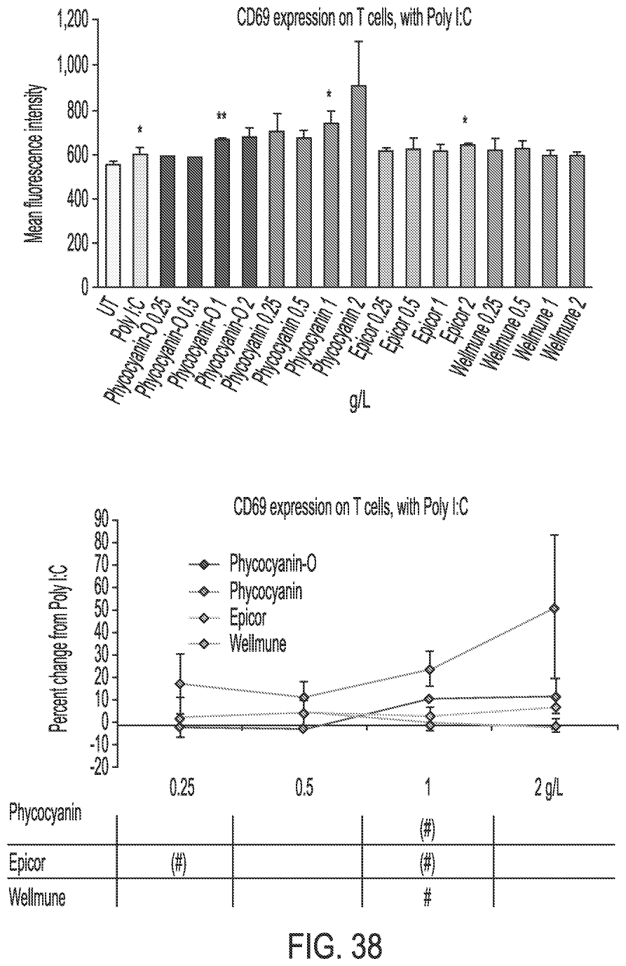

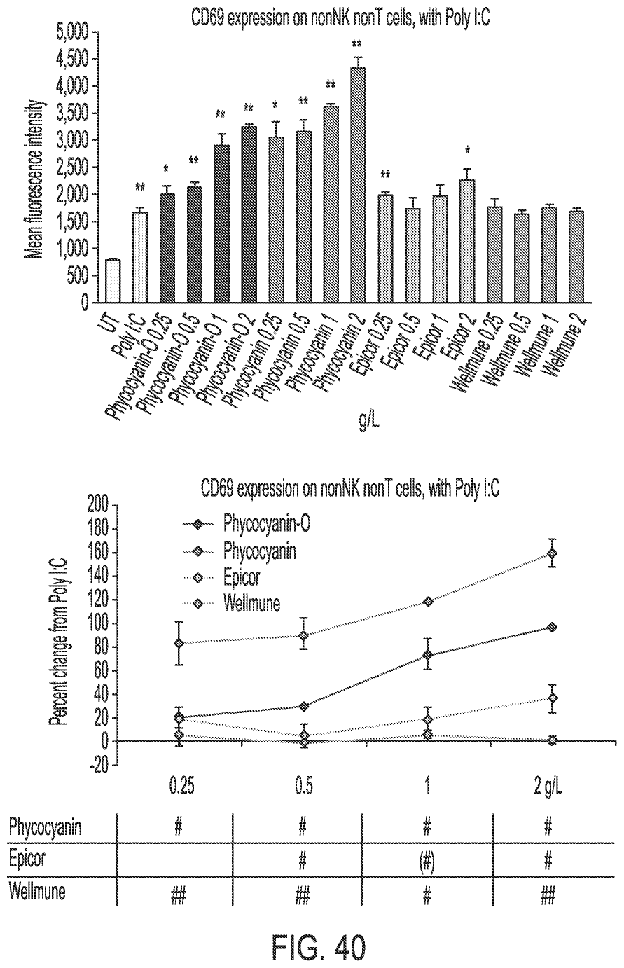

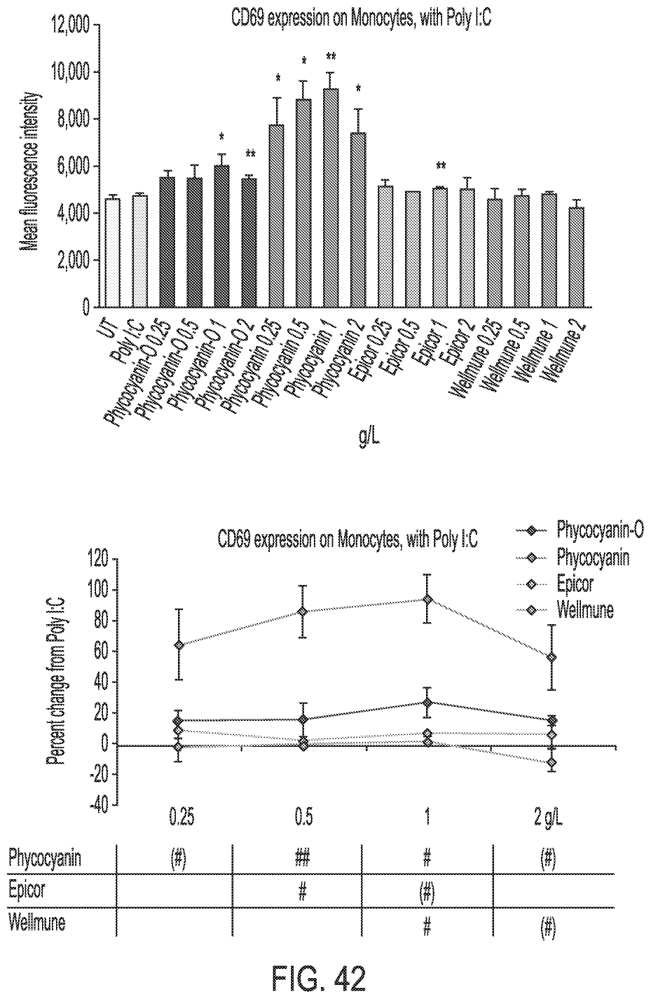

In context of an immune challenge with the viral mimetic Poly I:C, the Poly I:C-mediated increase in CD69 expression was further enhanced by O-CPs. The results relating to the expression of CD69 on NK cells is shown in FIG. 34. Expression of CD69 on NKT cells is shown in FIG. 36; expression of CD69 on T cells is shown in FIG. 38; and the expression of CD69 on non-NKT non-T cells is shown in FIG. 40. Monocytes: In the context of a Poly I:C-mediated immune challenge, both products enhanced the CD69 expression. The results are shown in FIG. 42.

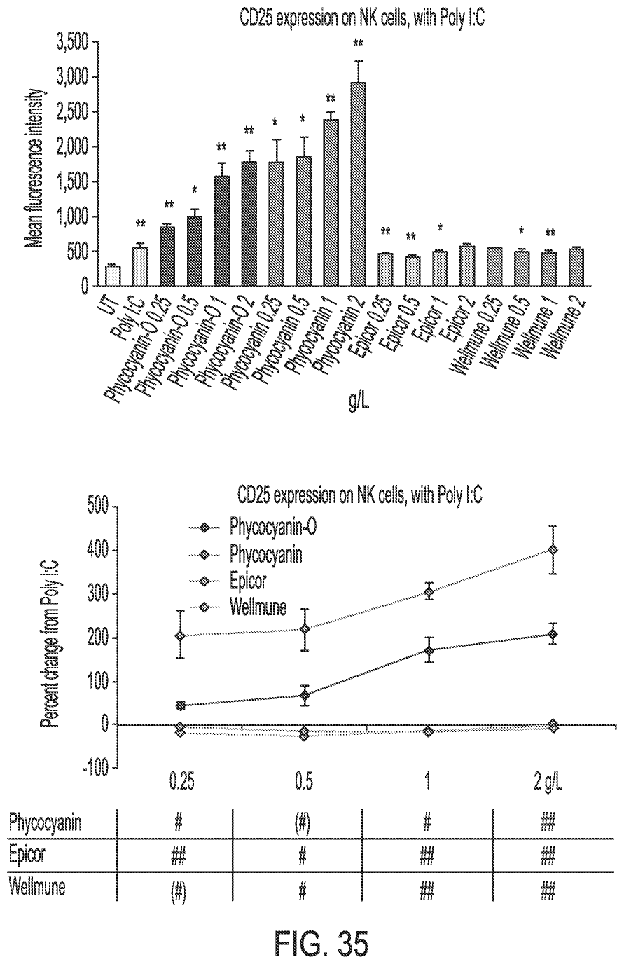

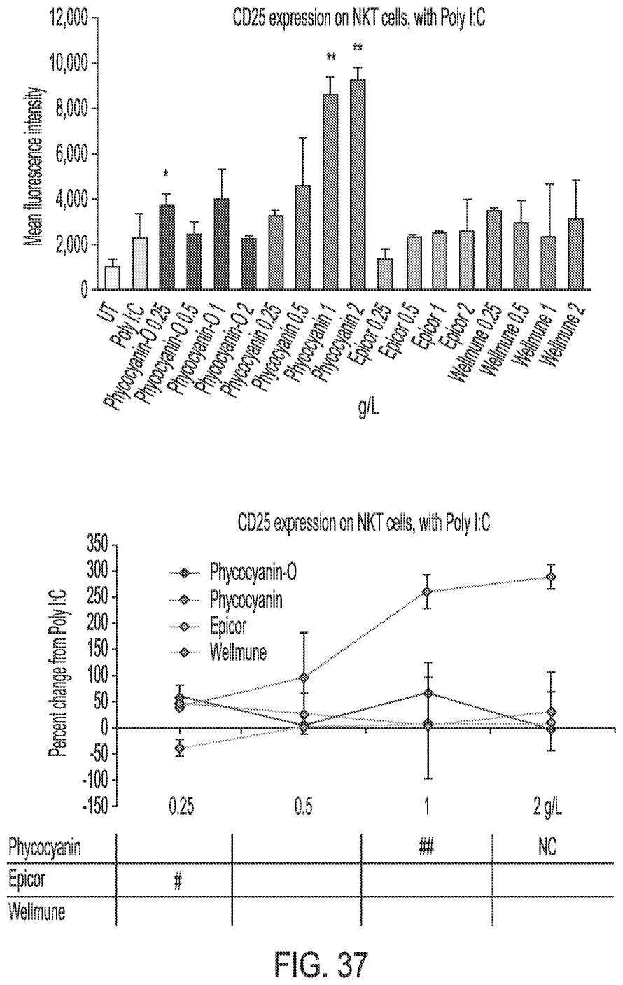

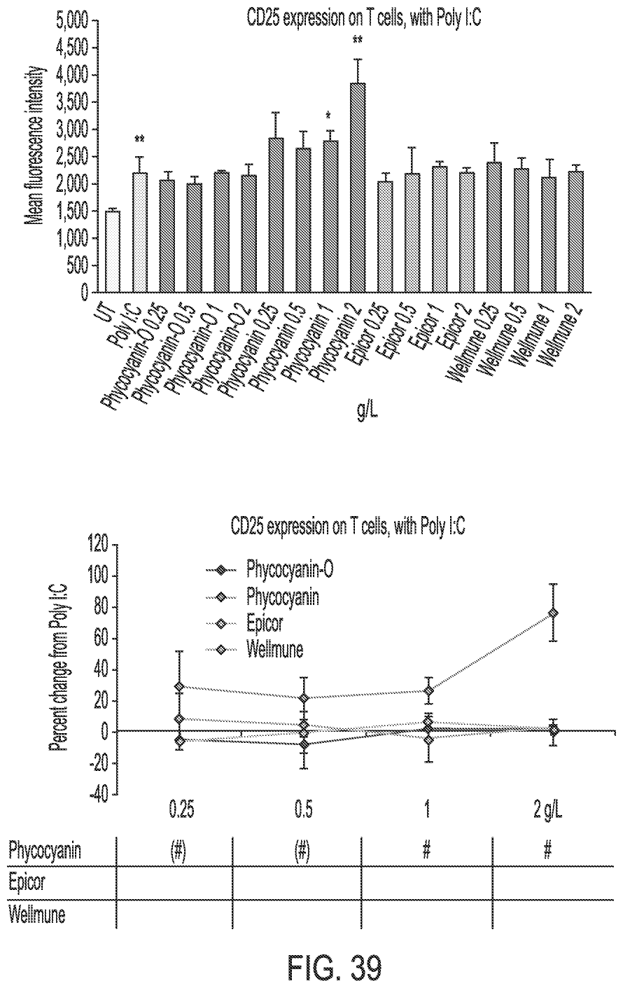

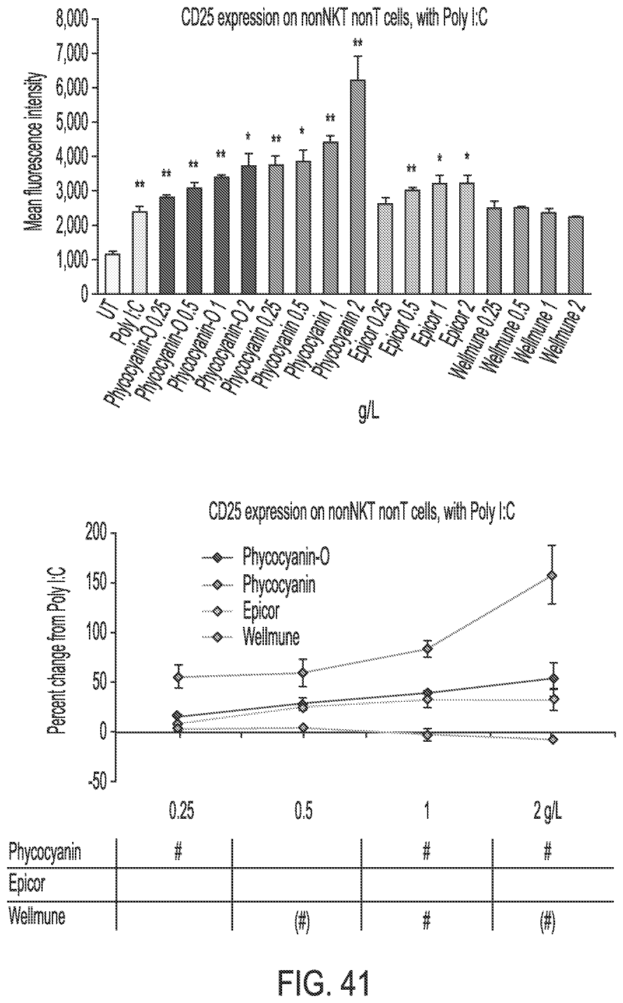

Effects on CD25 levels: In context of an immune challenge with the viral mimetic Poly I:C, the Poly I:C-mediated increase in CD25 expression on NK cells, NKT cells, and non-NK non-T cells was further enhanced by O-CPs. The results relating to the expression of CD25 on NK cells are shown in FIG. 35. Expression of CD25 on NKT cells are shown in FIG. 37; expression of CD25 on T cells are shown in FIG. 39; and the expression of CD25 on non-NKT non-T cells are shown in FIG. 41.

Comparing O-CPs to EpiCor and Wellmune (Aqueous Fraction): Effects on CD69 Levels:

In context of an immune challenge with the viral mimetic Poly I:C, the Poly I:C-mediated increase in CD69 expression was further enhanced by O-CPs but both EpiCor and Wellmune showed mostly different effects:
NK cells: EpiCor and Wellmune mildly reduced the LPS-mediated increase in CD69 expression as shown in FIG. 34.
NKT cells: Neither EpiCor nor Wellmune altered the LPS-induced CD69 expression to a level that was statistically significant, as shown in FIG. 36.
T lymphocytes: EpiCor only very mildly increased the Poly I:C-mediated increase in CD69 expression. Wellmune had no effect, as shown in FIG. 38.
Non-NK non-T cells: O-CPs increased CD69 expression much more than EpiCor. Wellmune had no effect, as shown in FIG. 40.
Monocytes: O-CPs triggered a slightly higher increase in CD69 expression than EpiCor. Wellmune had no effect, as shown in FIG. 42.

Effects on CD25 Levels:

In context of an inflammatory immune challenge with the viral mimetic Poly I:C, the Poly I:C-mediated increase in CD25 expression was further enhanced by O-CPs, but both EpiCor and Wellmune showed mostly different effects:
NK cells: Whereas O-CPs triggered strong enhancement of Poly I:C-medicated increases in CD25, both EpiCor and Wellmune triggered mild but statistically significant reductions in CD25 expression, as shown in FIG. 35.
NKT cells: Whereas O-CPs enhanced Poly I:C-medicated increases in CD25, neither EpiCor nor Wellmune affected CD25 expression, as shown in FIG. 37.
T lymphocytes: None of the 3 test products affected the Poly I:C-mediated increase in CD25, as shown in FIG. 39.
Non-NK non-T cells: O-CPs and EpiCor triggered similar increases in CD25, whereas Wellmune had no effect, as shown in FIG. 41.

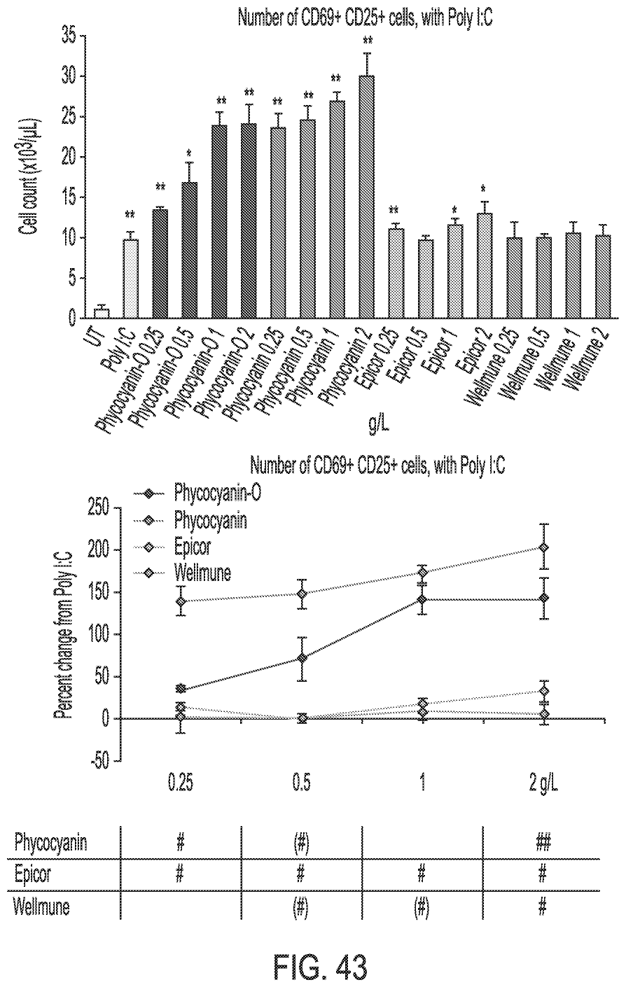

Effects on CD69+CD25+ cells: In the presence of a viral mimetic immune challenge, O-CPs stimulated an increase in CD69+CD25+ double-positive cells. EpiCor triggered a very mild increase, and Wellmune had no effect on the number of CD69+CD25+ cells, as shown in FIG. 43.

### Example 35

Changes to Cytokine Production by Algae-Based and Fermentation-Based Products

The culture supernatants were used for testing of a focused panel of 10 pro- and anti-inflammatory cytokines, using Luminex magnetic bead arrays and the MagPix® multiplexing system. The cytokine panel included: IL-1β, IL-1ra, IL-6, IL-8, IL-10, G-CSF, MIP-1α, MIP-1β, IFN-γ and TNF-α. This allowed documentation of 3 important steps in an immune reaction: Activation, restoration, and repair.

### Example 36

Direct Effects on Production of Immune Activating, Pro-Inflammatory Cytokines

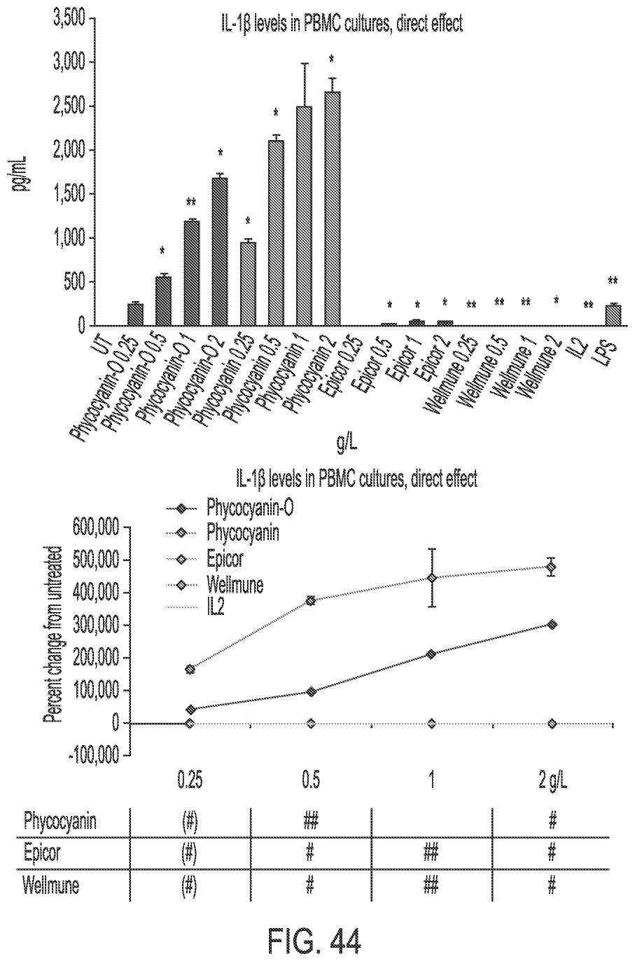

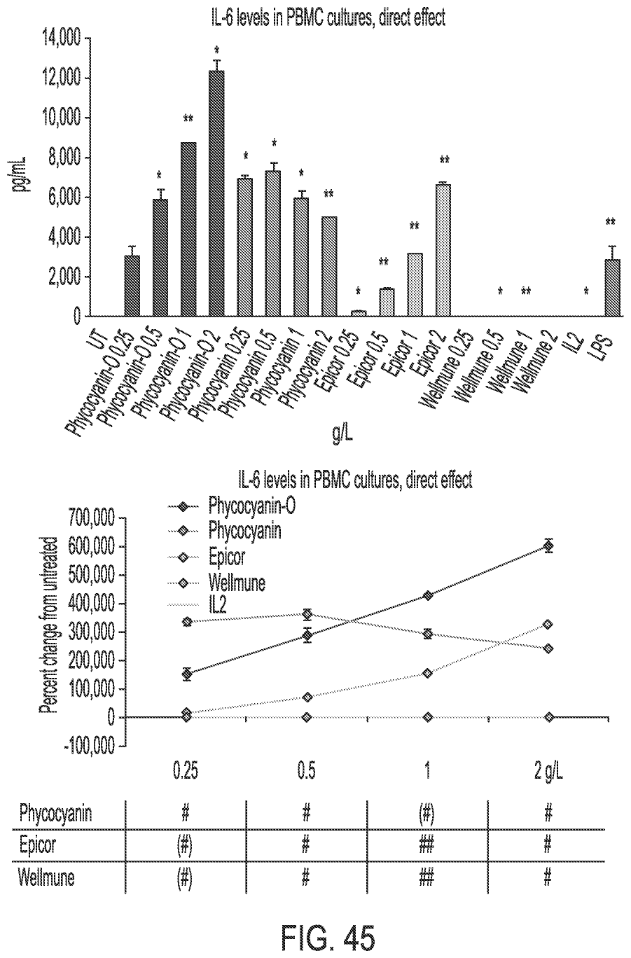

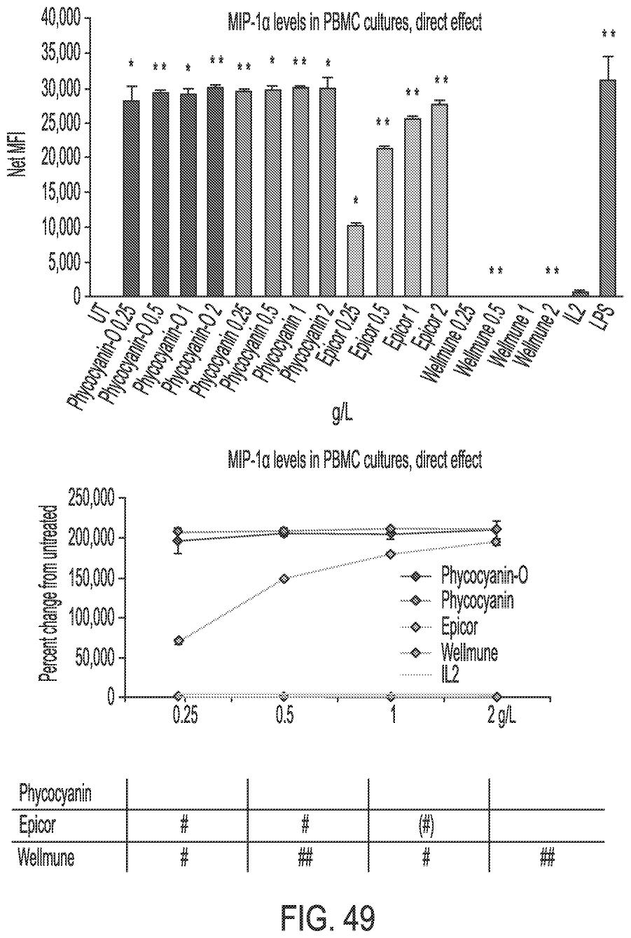

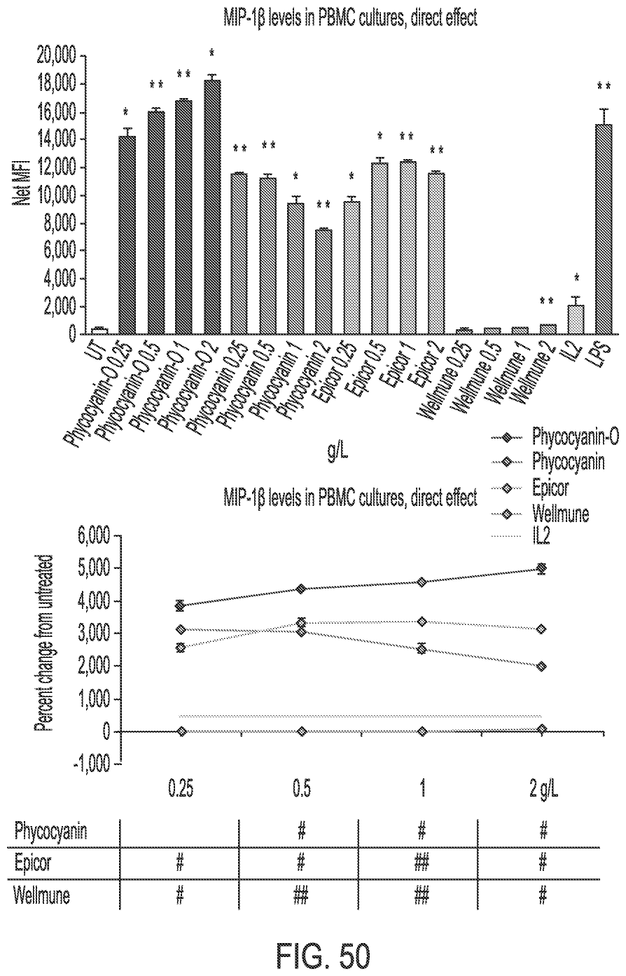

Aqueous Products:
O-CPs triggered higher levels of these cytokines when compared to PC: IL-6 and MIP-1β, as shown in FIGS. 45 and 50, respectively.
O-CPs increased MIP-1α, as shown in FIG. 49.
O-CPs triggered increases of IL-1β, IL-8, IFN-γ, and TNF-α, as shown in FIGS. 44, 46, 51 and 52, respectively.

### Example 37

Effects on Production of Immune Activating, Pro-Inflammatory Cytokines in Context of a Bacterial Inflammatory Challenge

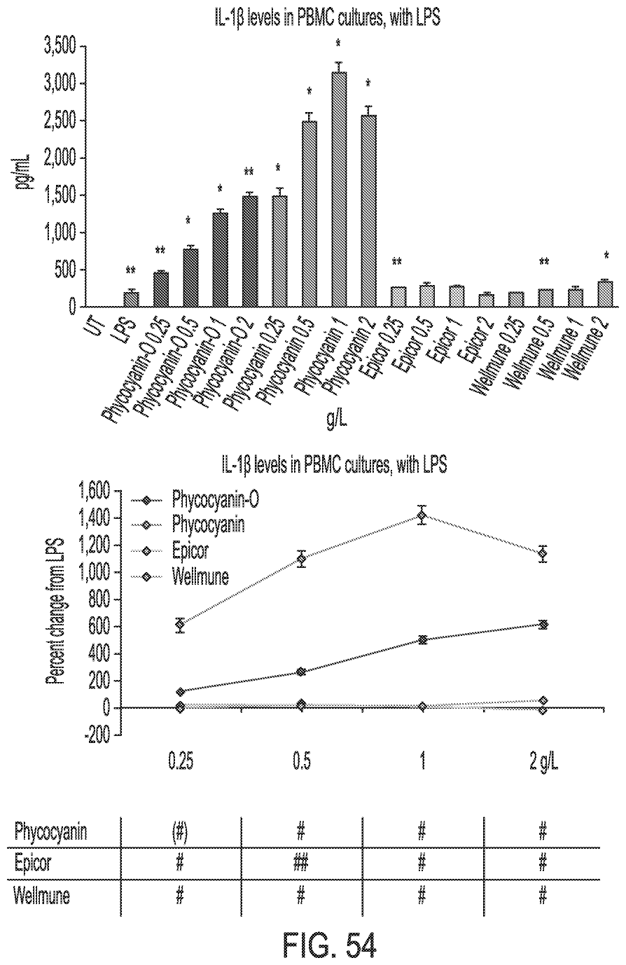

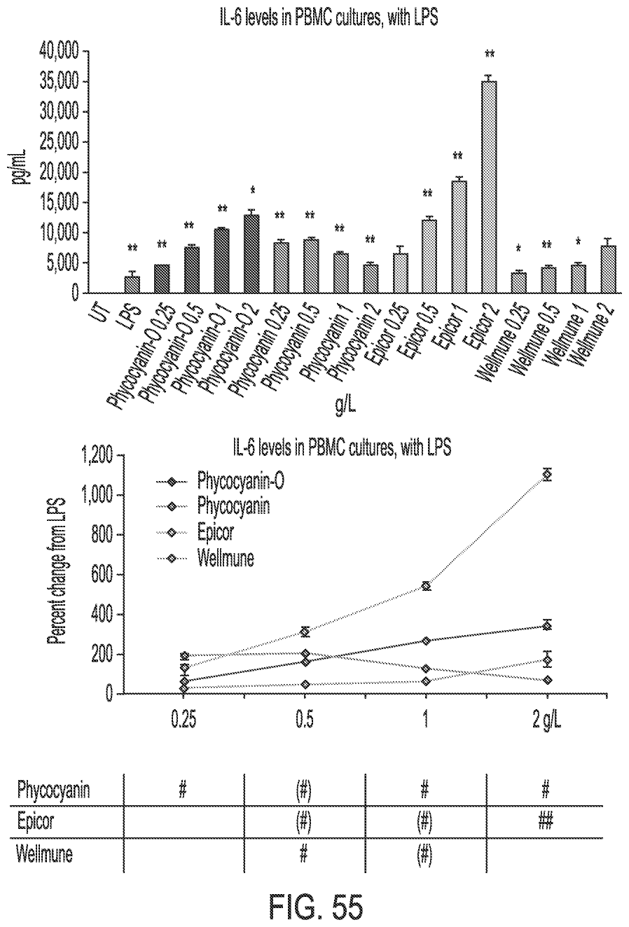

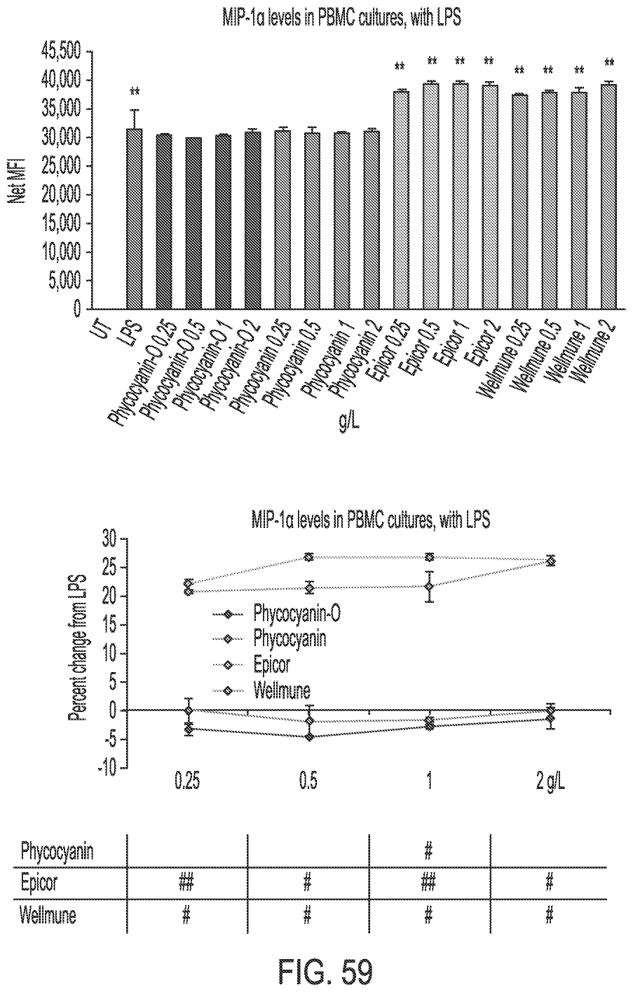

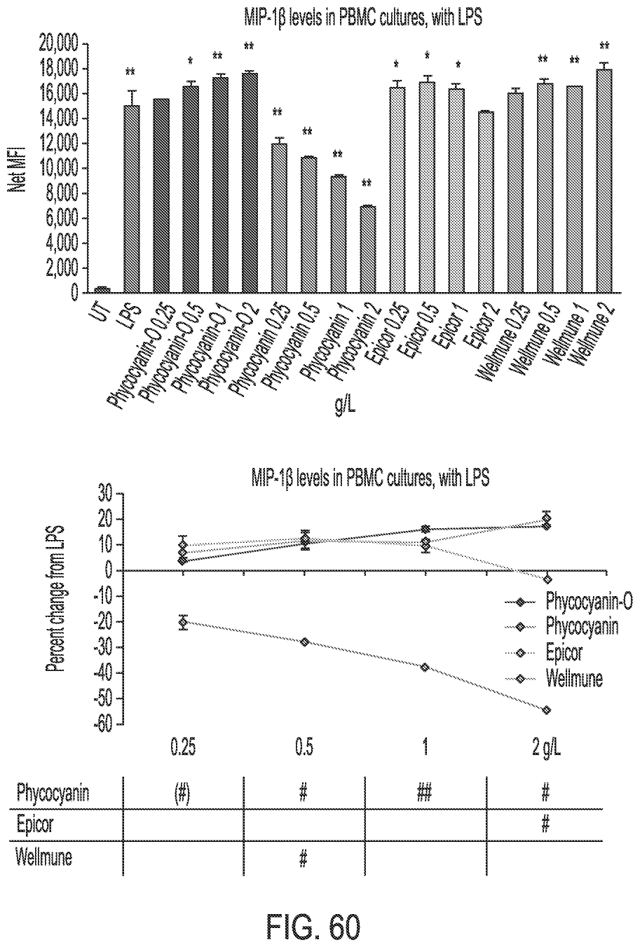

Aqueous Products
O-CPs triggered higher levels of IL-6 than PC, as shown in FIG. 55.
O-CPs triggered a decrease of MIP-1α, as shown in FIG. 59.
O-CPs triggered lower levels of these cytokines when compared to PC: IL-1β, IL-8, IFN-γ, and TNF-α, as shown in FIGS. 54, 56, 61, and 62, respectively.
O-CPs and PC triggered opposite effects on MIP-1(3: where O-CPs triggered an increase compared to the LPS control, as shown in FIG. 60.

### Example 38

Effects on Production of Immune Activating, Pro-Inflammatory Cytokines in Context of a Viral Mimetic Immune Challenge

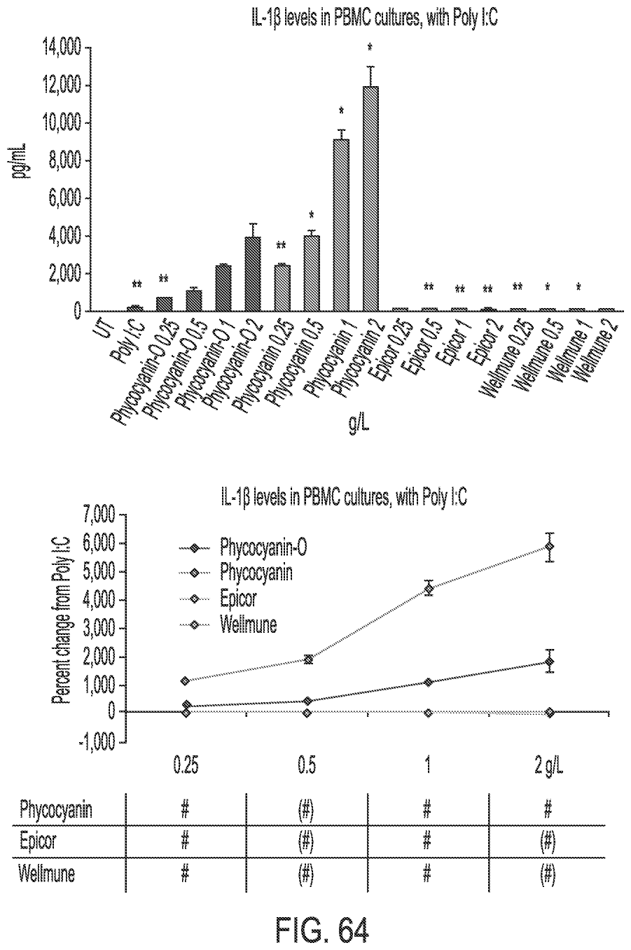

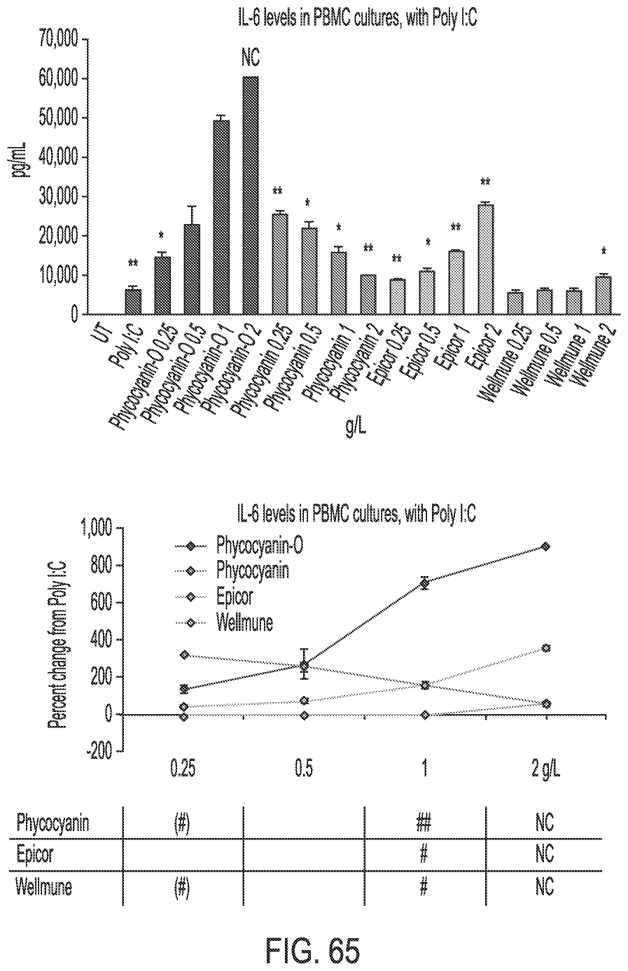

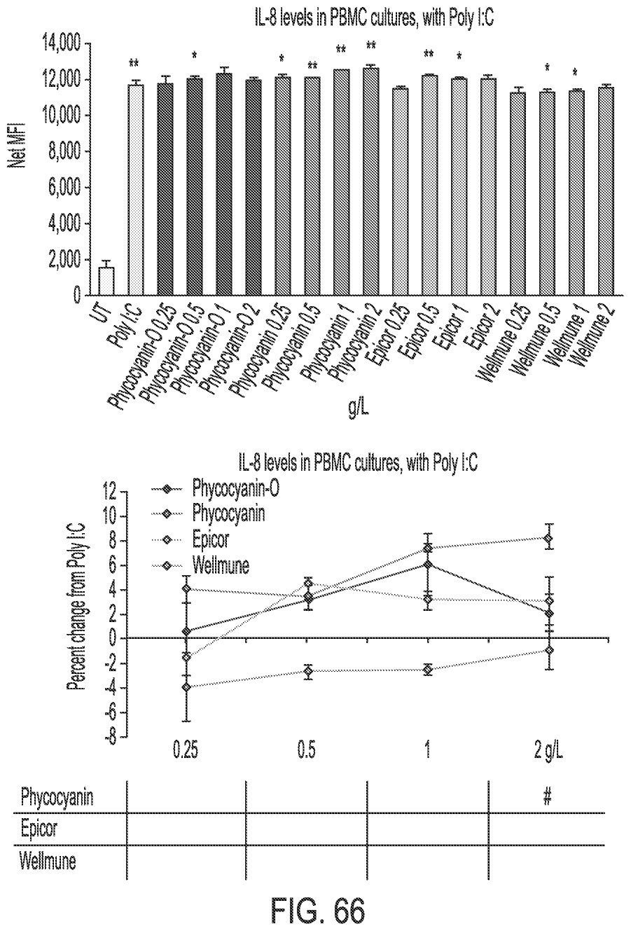

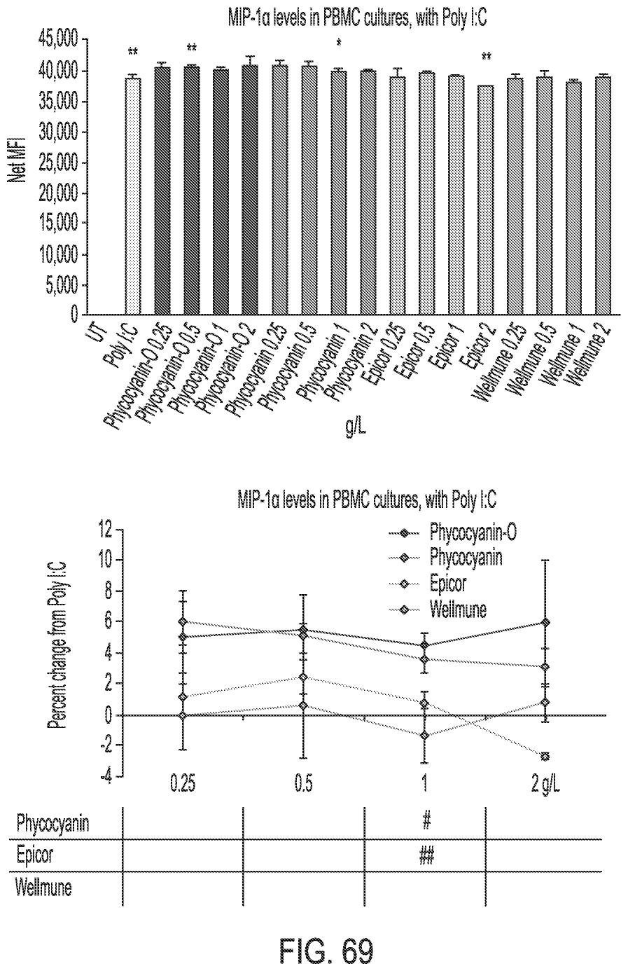

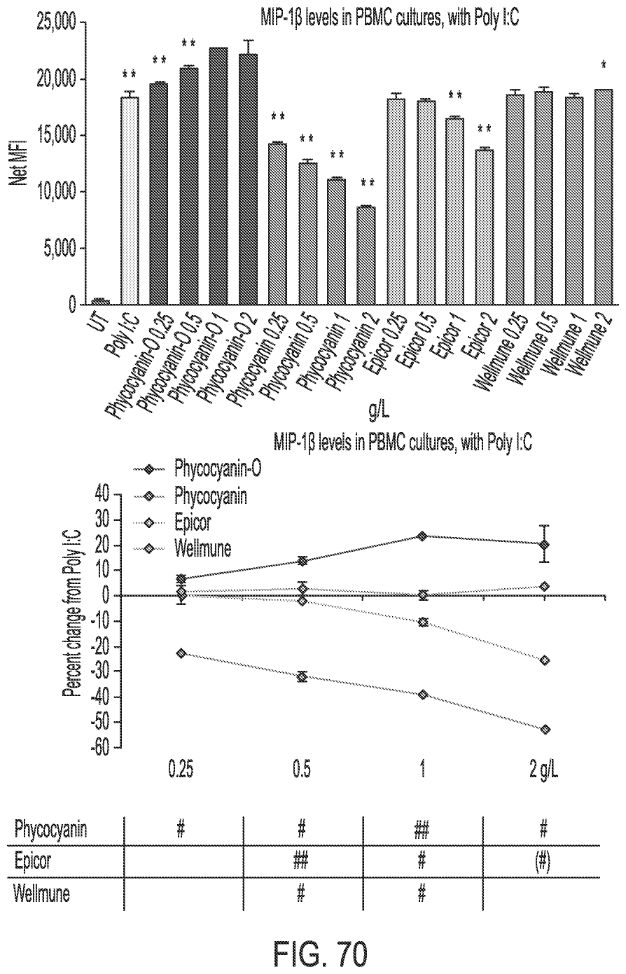

Aqueous Products
O-CPs triggered higher levels of IL-6 than PC, as shown in FIG. 65.
O-CPs increased IL-8 and MIP-1α, as shown in FIGS. 66 and 69, respectively.
O-CPs increased IL-1β, IFN-γ, and TNF-α, as shown in FIGS. 64, 71, and 72, respectively.
O-CPs and PC triggered opposite effects on MIP-1(3: where O-CPs triggered an increase, PC triggered a robust reduction compared to the LPS control, as shown in FIG. 70.
O-CPs triggered higher levels of IL-6 than EpiCor, whereas Wellmune had minimal effect on IL-6, as shown in FIG. 65.

### Example 39

Direct Effects on Production of the Stem Cell Activating Growth Factor G-CSF

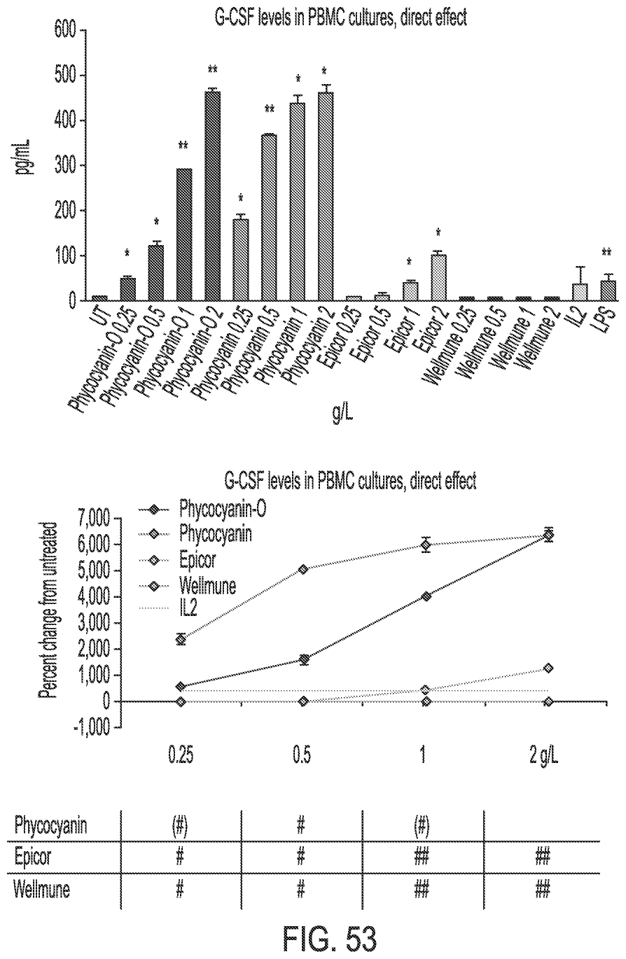

Aqueous Products
O-CPs directly triggered an increase in G-CSF levels; the increase was similar for the 2 products at the highest dose, but the effect at lower doses was weaker for O-CPs, as shown in FIG. 53.

### Example 40

Effects on Production of G-CSF in Context of a Bacterial Inflammatory Challenge

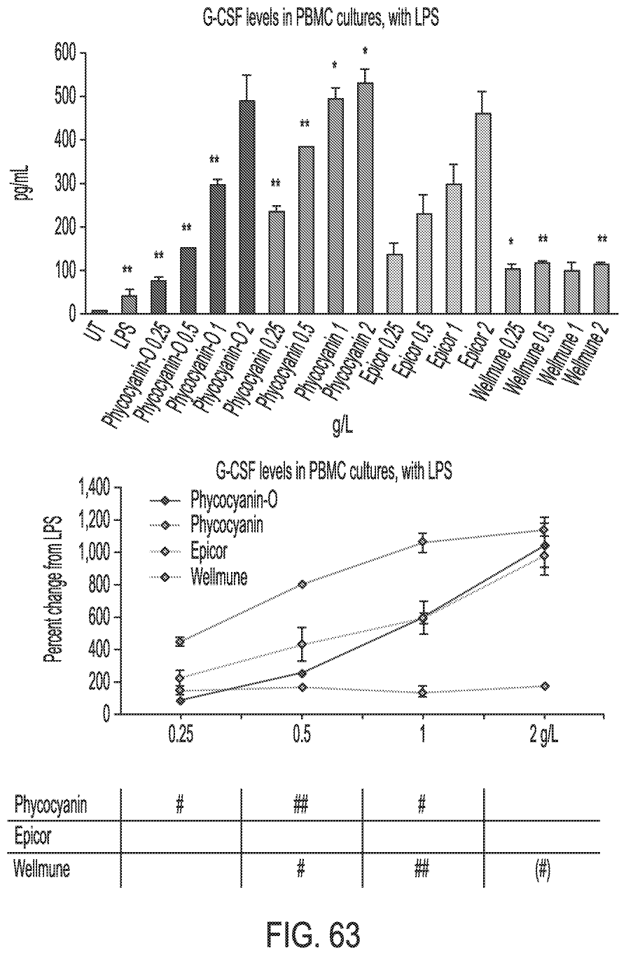

Aqueous Products
O-CPs directly triggered an increase in G-CSF levels, as shown in FIG. 63

### Example 41

Effects on Production of G-CSF in Context of a Viral Mimetic Immune Challenge

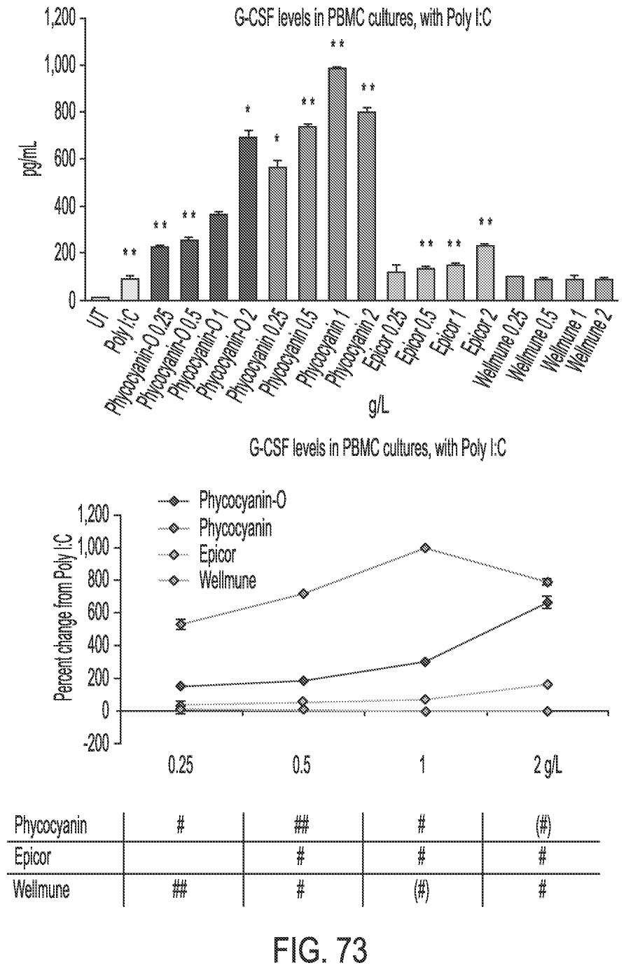

Aqueous Products
O-CPs directly triggered an increase in G-CSF levels, as shown in FIG. 73.

### Example 42

Algae-Based and Fermentation-Based Products

O-CPs had direct immune cell activating properties, and directly increased the expression of both CD69 and CD25 on all 4 types of lymphocytes analyzed. O-CPs had immune modulating properties, both in context of a bacterial inflammatory challenge, and in context of a viral mimetic challenge. O-CPs further enhanced the LPS-induced cell activation on all 4 types of lymphocytes analyzed. O-CPs also enhanced the Poly I:C-mediated immune cell activation on NK cells, NKT cells and non-NK non-T cells. O-CPs performed more robustly than the aqueous fraction of EpiCor. The aqueous fraction of Wellmune was fairly inactive, in terms of induction of cell activation markers. Table 7 shows the overview for effects of O-CPs on immune activation markers. In all the cases, O-CPs performed better than EpiCor and Wellmune.

TABLE 7
Overview for O-CPs effects on immune activation markers

| | | In context of | In context of viral |
|---|---|---|---|
| | Direct effect | inflammation | challenge |
| NK cells | CD69 ↑ | CD69 ↑ | CD69 ↑ |
| | CD25 ↑ | CD25 ↑ | CD25 ↑ |
| NKT cells | CD69 ↑ | CD69 ↑ | CD69 ↑ |
| | CD25 ↑ | CD25 ↑ | CD25 ↑ |
| T cells | CD69 ↑ | CD69 ↑ | CD69 ↑ |
| | CD25 ↑ | CD25 ↑ | CD25 no effect |
| Non-NK non-T cells | CD69 ↑ | CD69 ↑ | CD69 ↑ |
| | CD25 ↑ | CD25 ↑ | CD25 ↑ |
| CD69+ CD25+ cells | Numbers ↑ | Numbers ↑ | Numbers ↑ |
| Monocytes | CD69 no effect | CD69 ↑ | CD69 ↑ |

### Example 43

O-CPs and PC Displayed Differences in Terms of Effects on Cytokine Production

O-CPs had stronger effects on the up-regulation of IL-6, both in the absence of an immune challenge, and in context of a viral mimetic challenge. O-CPs had stronger effects on upregulation of MIP-1β under all 3 culture conditions. Interestingly, in the presence of the bacterial or viral immune challenge, O-CPs triggered an increase, in strong contrast to PC that triggered a very robust decrease.

In context of the bacterial or viral immune challenge, O-CPs increased IL-1ra levels. In context of the bacterial challenge that was an opposite effect compared to PC. In context of a viral mimetic challenge, both products increased IL-1ra, but the increase was stronger for O-CPs. Both products triggered increased IL-1β, TNF-α, IL-10 and G-CSF production under all 3 culture conditions. At a general level, O-CPs performed similarly or better than the aqueous fraction of EpiCor. Table 8 shows the overview for effects of O-CPs on cytokine production. O-CPs performed better than PC during expression levels of IL-6 and MIP-1β in absence of an immune challenge or direct effect, during expression levels of MIP-1β and IL-1ra in presence of an inflammatory immune challenge with the bacterial toxin LPS. O-CPs performed better than PC during expression levels of IL-6, MIP-1β and IL-1ra in presence of an immune challenge with the viral mimetic Poly I:C. O-CPs increased expression levels of IL-8, MIP-1α, and IL-1ra in case of direct effects.

TABLE 8
Overview for O-CPs effects on cytokine production

| | | In context of | In context of viral |
|---|---|---|---|
| | Direct effect | inflammation | challenge |
| Immune-activating, | IL-1b ↑ | IL-1b ↑ | IL-1b ↑ |
| pro-inflammatory | IL-6 ↑ | IL-6 ↑ | IL-6 ↑ |
| cytokines | IL-8 ↑ | IL-8 0 * | IL-8 ↑ |
| | MIP-α ↑ | MIP-α 0 | MIP-α ↑ |
| | MIP-1β ↑ | MIP-1β ↑ * | MIP-1β ↑ * |
| | TNF-α ↑ | TNF-α ↑ | TNF-α ↑ |
| | IFN-γ 0 | IFN-γ 0 | IFN-γ ↑ |
| Anti-inflammatory | IL-1ra ↑ | IL-1ra ↑ * | IL-1ra ↑ |
| cytokines | IL-10 ↑ | IL-10 ↑ | IL-10 ↑ |
| Growth factor | G-CSF ↑ | G-CSF ↑ | G-CSF ↑ |

* Opposite effects between O-CPs and PC

Based on the results described herein, further tests on immune activation and anti-inflammatory properties may be considered. Further work may also be considered where O-CPs is blended with zinc gluconate or zinc picolinate. The immune modulation work described herein was done once on cells from a healthy blood donor. Further validation repeats may be necessary for the immune activation and cytokine testing, using cells from an additional 2 healthy blood donors.

The present disclosure is not to be limited in terms of the particular embodiments described in this application, which are intended as illustrations of various aspects. Many modifications and variations can be made without departing from its spirit and scope. Functionally equivalent methods and apparatuses within the scope of the disclosure, in addition to those enumerated herein, are possible from the foregoing descriptions. Such modifications and variations are intended to fall within the scope of the appended claims. The present disclosure is to be limited only by the terms of the appended claims, along with the full scope of equivalents to which such claims are entitled. The terminology used herein is for the purpose of describing particular embodiments only and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

What is claimed is:

1. A composition comprising an amount of at least one oligo-chromopeptide for treating, preventing, or ameliorating one selected from the group consisting of age-related somatic disease, bacterial diseases or infections, viral diseases or infections, diseases associated with oxidative stress, symptoms associated with any of the foregoing, and any combination thereof.

2. The composition of claim 1, wherein the at least one oligo-chromopeptide has a molecular weight of less than 10 kDa.

3. The composition of claim 1, wherein the composition is formulated for delivery via an oral route, an intravenous route, a subcutaneous route, an intramuscular route, or an intraperitoneal route.

4. The composition of claim 1, wherein the composition further comprises an amount of a compound selected from the group consisting of nicotinamide riboside, selenium, a selenium compound, zinc, a zinc compound, and any combination thereof.

5. The composition of claim 1, wherein the composition further comprises an enzyme.

6. The composition of claim 5, wherein the enzyme is alcalase.

7. The composition of claim 1, wherein the composition is formulated as one selected from the group consisting of a solution, a suspension, an emulsion, a tablet, a pill, a pellet, a capsule, a caplet, a powder, a granule, a syrup, an elixir, a beverage, a suppository, an aerosol, a spray, and a chewable dosage form.

8. A method of making at least one oligo-chromopeptide from phycocyanin, the method comprising:
filtering a solution of crude phycocyanin, separating a protein fraction having a molecular weight more than 10 kDa from compounds having a molecular weight less than or equal to 10 kDa, and retaining the protein fraction having a molecular weight more than 10 kDa;
dissolving the protein fraction in a solvent at a concentration of about 50 g/L to about 300 g/L to make a protein solution;
adding an amount of enzyme to the protein solution;
adjusting the pH of the protein solution to a pH of about 6 to about 8;
adjusting the temperature of the protein solution to a temperature of about 50° C. to about 65° C.;
reacting the protein solution for about 2 hr to about 24 hr, wherein the reacting hydrolyzes proteins in the protein solution to make at least one oligo-chromopeptide having a molecular weight of less than 10 kDa; and
filtering the protein solution by filtration with a 10 kDa cutoff to obtain a permeate, wherein the permeate comprises the at least one oligo-chromopeptide.

9. The method of claim 8, wherein the enzyme is one selected from the group consisting of alcalase, neutrase, protamex, celluclast, viscozyme, and any combination thereof.

10. The method of claim 9, wherein the enzyme is alcalase.

11. The method of claim 9, wherein the amount of enzyme is about 0.002 g of enzyme per g of protein in the protein solution to about 1.0 g of enzyme per g of protein in the protein solution.

12. The method of claim 8, wherein the filtering a solution of crude phycocyanin is performed by ultrafiltration.

13. A method of treating, preventing, or ameliorating one or more diseases or conditions associated with oxidative stress, the method comprising:
identifying one or more diseases or conditions associated with oxidative stress in a subject;
administering to the subject a composition comprising an amount of at least one oligo-chromopeptide.

14. The method of claim 13, wherein the at least one oligo-chromopeptide has a molecular weight of less than 10 kDa.

15. The method of claim 13, wherein the composition further comprises an amount of a compound selected from the group consisting of nicotinamide riboside, selenium, a selenium compound, zinc, a zinc compound, and any combination thereof.

16. The method of claim 13, wherein the administering is performed via an oral route, an intravenous route, a subcutaneous route, an intramuscular route, or an intraperitoneal route.

17. A dietary supplement for treating, preventing, or ameliorating one selected from the group consisting of age-related somatic disease, bacterial diseases or infections, viral diseases or infections, diseases associated with oxidative stress, symptoms associated with any of the foregoing, and any combination thereof, wherein the dietary supplement comprises an amount of at least one oligo-chromopeptide

18. The dietary supplement of claim 17, wherein the at least one oligo-chromopeptide has a molecular weight of less than 10 kDa.

19. The dietary supplement of claim 17, wherein the dietary supplement further comprises an amount of a compound selected from the group consisting of nicotinamide riboside, selenium, a selenium compound, zinc, a zinc compound, and any combination thereof.

20. The dietary supplement of claim 17, wherein the dietary supplement is formulated as one selected from the group consisting of a solution, a suspension, an emulsion, a tablet, a pill, a pellet, a capsule, a caplet, a powder, a granule, a syrup, an elixir, a beverage, a suppository, an aerosol, a spray, and a chewable dosage form.
